SEQUENCE LISTING (16 sequences)

SEQ ID NO: 1 - protein, 205 aa, Human
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 2 - DNA, 20 bp, Artificial sequence
  sequence is synthesized
caaggccacctcctccccac

SEQ ID NO: 3 - DNA, 22 bp, Artificial sequence
  sequence is synthesized
tcttctctgggaaagcctactc

SEQ ID NO: 4 - DNA, 18 bp, Artificial sequence
  sequence is synthesized
cctcatgggccaggtaga

SEQ ID NO: 5 - DNA, 22 bp, Artificial sequence
  sequence is synthesized
acgtacaccctctcgcccctcc

SEQ ID NO: 6 - DNA, 18 bp, Artificial sequence
  sequence is synthesized
acgggcctctctggtaca

SEQ ID NO: 7 - DNA, 21 bp, Artificial sequence
  sequence is synthesized
catatcggggtgactgatgtt

SEQ ID NO: 8 - DNA, 21 bp, Artificial sequence
  sequence is synthesized
ctgaggcctctgctccccagg

SEQ ID NO: 9 - DNA, 21 bp, Artificial sequence
  sequence is synthesized
tggtgaccaactgtcactcat

SEQ ID NO: 10 - DNA, 24 bp, Artificial sequence
  sequence is synthesized
aatagtaggccgattacagacaca

SEQ ID NO: 11 - DNA, 25 bp, Artificial sequence
  sequence is synthesized
cacaagctgaaggcagacaaggccc

SEQ ID NO: 12 - DNA, 18 bp, Artificial sequence
  sequence is synthesized
gcggattctcatggaaca

SEQ ID NO: 13 - DNA, 20 bp, Artificial sequence
  sequence is synthesized
ggtcagccaggagcttcttg

SEQ ID NO: 14 - DNA, 24 bp, Artificial sequence
  sequence is synthesized
aactgcaccttcacacagagctgc

SEQ ID NO: 15 - DNA, 20 bp, Artificial sequence
  sequence is synthesized
ctctcttggcagccttcctg

SEQ ID NO: 16 - DNA, 24 bp, Artificial sequence
  sequence is synthesized
ctaagttctttagcactccttggc

(19) United States
(12) Patent Application Publication
(10) Pub. No.: US 2011/0263451 A1
(43) Pub. Date: Oct. 27, 2011

(54) BIOLOGICAL MARKERS PREDICTIVE OF RHEUMATOID ARTHRITIS RESPONSE TO LYMPHOTOXIN ANTAGONISTS

(75) Inventors: Jane Grogan, San Francisco, CA (US); Wai Lee Wong, Los Altos, CA (US); Judy Young, San Carlos, CA (US)

(73) Assignee: GENENTECH, INC

(21) Appl. No.: 13/121,675

(22) PCT Filed: Sep. 29, 2009
(86) PCT No.: PCT/US09/58797

§ 371 Date: Jul. 14, 2011

(30) Foreign Application Priority Data
Sep. 30, 2008 (US) 61194850
May 7, 2009 (US) 61176406

(51) Int. Cl. C40B 30/04; C07K 14/52

(52) U.S. Cl. 506/9; 530/351

(57) ABSTRACT

The present invention relates to a soluble lymphotoxin (solLT) and methods of using the solLT as a biomarker in the treatment of autoimmune disease. More particularly, the present invention relates to soluble lymphotoxin alpha-beta (solLTαβ) and methods of using this solLTαβ as a biomarker in the treatment of rheumatoid arthritis (RA).

### CROSS-REFERENCE TO RELATED APPLICATIONS

The present application claims the benefit of and priority to U.S. Provisional Application Ser. No. 61/194,850, filed Sep. 30, 2008 (Attorney Docket No. PR4254) and U.S. Provisional Application Ser. No. 61/176,406, filed May 7, 2009 (Attorney Docket No. PR4254-1), the entire disclosures of which are incorporated herein by reference.

### FIELD OF THE INVENTION

The present invention relates to a soluble lymphotoxin (solLT) and methods of using the solLT as a biomarker in the treatment of autoimmune disease. More particularly, the present invention relates to soluble lymphotoxin alpha-beta (solLTαβ) and methods of using this solLTαβ as a biomarker in the treatment of rheumatoid arthritis (RA).

### BACKGROUND OF THE INVENTION

Autoimmune diseases remain clinically important diseases in humans. As the name implies, autoimmune diseases act through the body's own immune system. While the pathological mechanisms differ among individual types of autoimmune diseases, one general mechanism involves the generation of antibodies (referred to herein as self-reactive antibodies or autoantibodies) directed against specific endogenous proteins. Physicians and scientists have identified more than 70 clinically distinct autoimmune diseases, including RA, multiple sclerosis, vasculitis, immune-mediated diabetes, and lupus such as SLE. While many autoimmune diseases are rare—affecting fewer than 200,000 individuals—collectively, these diseases afflict millions of Americans, an estimated five percent of the population, with women disproportionately affected by most diseases. The chronic nature of these diseases leads to an immense social and financial burden.

Inflammatory arthritis is a prominent clinical manifestation in diverse autoimmune disorders including rheumatoid arthritis (RA), psoriatic arthritis (PsA), systemic lupus erythematosus (SLE), Sjögren's syndrome, and polymyositis. Most of these patients develop joint deformities on physical examination but typically only RA and PsA patients manifest bone erosions on imaging studies.

RA is a chronic inflammatory disease that affects approximately 0.5 to 1% of the adult population in northern Europe and North America, and a slightly lower proportion in other parts of the world (Alamanosa and Drosos, Autoimmun. Rev., 4:130-136 (2005)). It is a systemic inflammatory disease characterized by chronic inflammation in the synovial membrane of affected joints, which ultimately leads to loss of daily function due to chronic pain and fatigue. The majority of patients also experience progressive deterioration of cartilage and bone in the affected joints, which may eventually lead to permanent disability. The long-term prognosis of RA is poor, with approximately 50% of patients experiencing significant functional disability within 10 years from the time of diagnosis (Keystone, Rheumatology, 44 (Suppl. 2):ii8-ii12 (2005)). Life expectancy is reduced by an average of 3-10 years (Alamanosa and Drosos, supra). Patients with a high titer of rheumatoid factor (RF) (approximately 80% of patients) have more aggressive disease (Bukhari et al., Arthritis Rheum. 46:906-912 (2002)), with a worse long-term outcome and increased mortality over those who are RF negative (Heliovaara et al., Ann. Rheum. Dis., 54:811-814 (1995)).

The pathogenesis of chronic inflammatory bone diseases, such as RA, is not fully elucidated. Such diseases are accompanied by bone loss around affected joints due to increased osteoclastic resorption. This process is mediated largely by increased local production of pro-inflammatory cytokines (Teitelbaum, Science, 289:1504-1508 (2000); Goldring, Arthritis Res. 2(1):33-37 (2000)). These cytokines can act directly on cells in the osteoclast lineage or indirectly by affecting the production of the essential osteoclast differentiation factor, receptor activator of NFκB ligand (RANKL), and/or its soluble decoy receptor, osteoprotegerin (OPG), by osteoblast/stromal cells (Hossbauer et al., J. Bone Miner. Res., 15(1):2-12 (2000)). Tumor necrosis factor-alpha (TNF-α) is a major mediator of inflammation, whose importance in the pathogenesis of various forms of bone loss is supported by several lines of experimental and clinical evidence (Feldmann et al., Cell, 85(3):307-310 (1996)). However, TNF-α is not essential for osteoclastogenesis (Douni et al., J. Inflamm., 47:27-38 (1996)), erosive arthritis (Campbell et al., J. Clin. Invest., 107(12):1519-1527 (2001)), or osteolysis (Childs et al., J. Bon. Min. Res., 16:338-347 (2001)), as these can occur in the absence of TNF-α.

Tumor Necrosis Factor (TNF)-related proteins are recognized in the art as a large family of proteins having a variety of activities ranging from host defense to immune regulation to apoptosis. Many tumor necrosis factor superfamily (TNF-SF) members are among those elevated. The TNF-SF is a large family of eighteen identified members that exhibit a variety of activities ranging from host defence to immune regulation to apoptosis (Locksley et al., Cell 2001; 104(4):487-501). Members of the TNF-SF exist in membrane-bound forms that act locally through cell-cell contact, or as secreted proteins. A family of TNF-SF receptors (TNFR-SF) bind these proteins and triggers a variety of signalling pathways including apoptosis, cell proliferation, tissue differentiation, and pro-inflammatory responses. TNF-α by itself has been implicated in inflammatory diseases; autoimmune diseases; viral, bacterial, and parasitic infections, malignancies, and neurodegenerative diseases and is a specific therapeutic target in autoimmune diseases such as RA and Crohn's disease (Feldmann et al., 2001, supra).

In RA specifically, an immune response is thought to be initiated/perpetuated by one or several antigens presenting in the synovial compartment, producing an influx of acute inflammatory cells and lymphocytes into the joint. Successive waves of inflammation lead to the formation of an invasive and erosive tissue called pannus. This contains proliferating fibroblast-like synoviocytes and macrophages that produce proinflammatory cytokines such as TNF-α and interleukin-1 (IL-1). Local release of proteolytic enzymes, various inflammatory mediators, and osteoclast activation contribute to much of the tissue damage. There is loss of articular cartilage and the formation of bony erosions. Surrounding tendons and bursa may become affected by the inflammatory process. Ultimately, the integrity of the joint structure is compromised, producing disability.

The precise contributions of B cells to the immunopathogenesis of RA are not completely characterized. However, there are several possible mechanisms by which B cells may participate in the disease process (Silverman and Carson, Arthritis Res. Ther., 5 Suppl. 4:S1-6 (2003)).

Historically, B cells were thought to contribute to the disease process in RA predominantly by serving as the precursors of autoantibody-producing cells. A number of autoantibody specificities have been identified including antibodies to Type II collagen, and proteoglycans, as well as rheumatoid factors. The generation of large quantities of antibody leads to immune complex formation and the activation of the complement cascade. This in turn amplifies the immune response and may culminate in local cell lysis. Increased RF synthesis and complement consumption has been correlated with disease activity. The presence of RF itself is associated with a more severe form of RA and the presence of extra-articular features.

Recent evidence (Janeway and Katz, J. Immunol., 138:1051 (1998); Rivera et al., Int. Immunol., 13:1583-1593 (2001)) shows that B cells are highly efficient antigen-presenting cells (APC). RF-positive B cells may be particularly potent APCs, since their surface immunoglobulin would readily allow capture of any immune complexes regardless of the antigens present within them. Many antigens may thus be processed for presentation to T cells. In addition, it has been recently suggested that this may also allow RF-positive B cells to self-perpetuate (Edwards et al., Immunology, 97:188-196 (1999)).

For activation of T cells, two signals need to be delivered to the cell; one via the T-cell receptor (TCR), which recognizes the processed peptide in the presence of major histocompatibility complex (MHC) antigen, and a second, via co-stimulatory molecules. When activated, B cells express co-stimulatory molecules on their surface and can thus provide the second signal for T-cell activation and the generation of effector cells.

B cells may promote their own function as well as that of other cells by producing cytokines (Harris et al., Nat. Immunol., 1:475-482 (2000)). TNF-α and IL-1, lymphotoxin-alpha (LTα), interleukin-6 (IL-6), and interleukin-10 (IL-10) are amongst some of the cytokines that B cells may produce in the RA synovium.

Although T-cell activation is considered to be a key component in the pathogenesis of RA, recent work using human synovium explants in severe combined immunodeficiency disorders (SCID) mice has demonstrated that T-cell activation and retention within the joint is critically dependent on the presence of B cells (Takemura et al., J. Immunol., 167:4710-4718 (2001)).

The precise role of B cells in this is unclear, since other APCs did not appear to have the same effect on T cells.

Structural damage to joints is an important consequence of chronic synovial inflammation. Between 60% and 95% of patients with RA develop at least one radiographic erosion within 3-8 years of disease onset (Paulus et al., J. Rheumatol., 23:801-805 (1996); Hulsmans et al., Arthritis Rheum., 43:1927-1940 (2000)). In early RA, the correlation between radiographic damage scores and functional capacity is weak, but after 8 years of disease, correlation coefficients can reach as high as 0.68 (Scott et al., Rheumatology, 39:122-132 (2000)). In 1,007 patients younger than age 60 years who had RA for at least four years, Wolfe et al. (Arthritis Rheum, 43 Suppl. 9:S403 (2000)) found a significant association between the rate of progression of the Larsen radiographic damage score (Larsen et al., Acta Radiol. Diagn. 18:481-491 (1977)), increasing social security disability status, and decreasing family income.

Prevention or retardation of radiographic damage is one of the goals of RA treatment (Edmonds et al., Arthritis Rheum., 36:336-340 (1993)). Controlled clinical trials of 6 or 12 months' duration have documented that the progression of radiographic damage scores was more rapid in the placebo group than in groups that received methotrexate (MTX) (Sharp et al., Arthritis Rheum., 43:495-505 (2000)), leflunomide (Sharp et al., supra), sulfasalazine (SSZ) (Sharp et al., supra), prednisolone (Kirwan et al., N. Engl. J. Med., 333:142-146 (1995); Wassenburg et al., Arthritis Rheum, 42:Suppl 9:S243 (1999)), interleukin-1 receptor antagonist (Bresnihan et al., Arthritis Rheum, 41:2196-2204 (1998)), or an infliximab/MTX combination (Lipsky et al., N. Eng. J. Med., 343:1594-1604 (2000)), and that radiographic progression following treatment with etanercept was less rapid than that following treatment with MTX (Bathon et al., N. Engl. J. Med., 343:1586-1593 (2000)). Other studies have evaluated radiographic progression in patients treated with corticosteroids (Joint Committee of the Medical Research Council and Nuffield Foundation, Ann Rheum. Dis., 19:331-337 (1960); Van Everdingen et al., Ann. Intern. Med., 136:1-12 (2002)), cyclosporin A (Pasero et al., J. Rheumatol., 24:2113-2118 (1997); Forre, Arthritis Rheum., 37:1506-1512 (1994)), MTX versus azathioprine (Jeurissen et al., Ann. Intern. Med., 114:999-1004 (1991)), MTX versus auranofin (Weinblatt et al., Arthritis Rheum., 36:613-619 (1993)), MTX (meta-analysis) (Alarcon et al., J. Rheumatol., 19:1868-1873 (1992)), hydroxychloroquine (HCQ) versus SSZ (Van der Heijde et al., Lancet, 1:1036-1038), SSZ (Hannonen et al., Arthritis Rheum., 36:1501-1509 (1993)), the COBRA (Combinatietherapei Bij Reumatoide Artritis) combination of prednisolone, MTX, and SSZ (Boers et al., Lancet, 350:309-318 (1997); Landewe et al., Arthritis Rheum., 46:347-356 (2002)), combinations of MTX, SSZ, and HCQ (O'Dell et al., N. Engl. J. Med., 334:1287-1291 (1996); Mottonen et al., Lancet, 353:1568-1573 (1999)), the combination of cyclophosphamide, azathioprine, and HCQ (Csuka et al., JAMA, 255:2115-2119 (1986)), and the combination of adalimumab with MTX (Keystone et al., Arthritis Rheum., 46 Suppl. 9:S205 (2002)).

The FDA has now approved labeling claims that certain medications, e.g., leflunomide, etanercept, and infliximab, slow the progression of radiographic joint damage. These claims are based on the statistically significant differences in progression rates observed between randomly assigned treatment groups and control groups. However, the progression rates in individuals within the treatment and control groups overlap to a considerable extent; therefore, despite significant differences between treatment groups, these data cannot be used to estimate the probability that a patient who is starting a treatment will have a favorable outcome with respect to progression of radiographic damage. Various methods have been suggested to categorize paired radiographs from individual patients as not progressive, e.g., damage scores of 0 at both time points, no increase in damage scores, no new joints with erosions, and a change in score not exceeding the smallest detectable difference (i.e., 95% confidence interval for the difference between repeated readings of the same radiograph) (Lassere et al., J. Rheumatol., 26:731-739 (1999)).

Determining whether there has been increased structural damage in an individual patient during the interval between paired radiographs obtained at the beginning and end of a 6- or 12-month clinical trial has been difficult, for several reasons. The rate of radiographic damage is not uniform within a population of RA patients; a few patients may have rapidly progressing damage, but many may have little or no progression, especially if the tie interval is relatively short. The methods for scoring radiographic damage, e.g., Sharp (Sharp et al., Arthritis Rheum., 14:706-720 (1971); Sharp et al., Arthritis Rheum., 28:1326-1335 (1985)), Larsen (Larsen et al., Acta Radiol. Diagn., 18:481-491 (1977)), and modifications of these methods (Van der Heijde, J. Rheumatol., 27:261-263 (2000)), depend on the judgment and the interpretation of the reader as to whether an apparent interruption of the subchondral cortical plate is real, or whether a decrease in the distance between the cortices on opposite sides of a joint is real or is due to a slight change in the position of the joint relative to the film and the radiographic beam, to a change in radiographic exposure, or to some other technical factor.

Therefore, the recorded score is an approximation of the true damage, and for many subjects, the smallest detectable difference between repeat scores of the same radiographs is larger than the actual change that has occurred during the interval between the baseline and final radiographs. If the reader is blinded to the temporal sequence of the films, these unavoidable scoring errors may be in either direction, leading to apparent “healing” when the score decreases or to apparent rapid progression when reading error increases the difference between films. When the study involves a sufficiently large population of patients who have been randomly assigned to receive an effective treatment as compared with placebo, the positive and negative reading errors offset each other, and small but real differences between treatment groups can be detected.

The imprecision of the clinical measures that are used to quantitate RA disease activity has caused a similar problem; statistically significant differences between certain outcome measures from clinical trials were not useful for estimating the probability of improvement for an individual who was starting the treatment (Paulus et al., Arthritis Rheum., 33:477-484 (1990)). Attribution of individual improvement became practical with the creation of the American College of Rheumatology (ACR) 20% composite criteria for improvement (ACR20), which designated a patient as improved if there was 20% improvement in the tender and swollen joint counts and 20% improvement in at least 3 of 5 additional measures (pain, physical function, patient global health assessment, physician global health assessment, and acute-phase reactant levels) (Felson et al., Arthritis Rheum., 38:727-735 (1995)). All of these measures have large values for the smallest detectable difference, but by requiring simultaneous improvement in 5 of the 7 aspects of the same process (disease activity), the randomness of the 7 measurement errors is constrained and it is easier to attribute real improvement to the individual.

In RA, joint damage is a prominent feature. Radiologic parameters of joint destruction are seen as a key outcome measure in descriptions of disease outcome. In the recent OMERACT (Outcome Measures in Rheumatology Clinical Trials) consensus meeting, radiology was chosen as part of the core set of outcome measures for longitudinal observational studies (Wolfe et al., Arthritis Rheum., 41 Supp 9:S204 (1998) abstract). Radiology is also part of the WHO/ILAR (World Health Organization/International League of Associations for Rheumatology) required core set of measures for long-term clinical trials (Tugwell and Boers, J. Rheumatol., 20:528-530 (1993)).

Available data on the outcome of radiologic damage in RA have been obtained in both short-term and long-term studies. In short-term studies of RA patients with recent-onset disease, radiographs obtained every 6 months showed that after an initial rapid progression, there was diminution of the progression rate of radiologic damage in the hands and feet after 2-3 years (Van der Heijde et al., Arthritis Rheum., 35:26-34 (1992); Fex et al., Br. J. Rheumatol., 35:1106-1055 (1996)). In long-term studies with radiographs taken less frequently, a constant rate of progression was found, with relentless deterioration of damage up to 25 years of disease duration (Wolfe and Sharp, Arthritis Rheum., 41:1571-1582 (1998); Graudal et al., Arthritis Rheum., 41:1470-1480 (1998); Plant et al., J. Rheumatol., 25:417-426 (1998); Kaarela and Kautiainen, J. Rheumatol., 24:1285-1287 (1997)). Whether these differences in radiographic progression pattern are due to differences in the scoring techniques is not clear.

The scoring systems used differ in the number of joints being scored, the presence of independent scores for erosions (ERO) and joint space narrowing (JSN), the maximum score per joint, and the weighing of a radiologic abnormality. As yet, there is no consensus on the scoring method of preference. During the first 3 years of follow-up in a cohort study of patients with early arthritis, JSN and ERO were found to differ in their contribution to the measured progression in radiologic damage of the hands and feet (Van der Heijde et al., Arthritis Rheum., 35:26-34 (1992)). Furthermore, methods that independently score ERO and JSN, such as the Sharp and Kellgren scores, were found to be more sensitive to change in early RA than methods using an overall measure, such as the Larsen score (Plant et al., J. Rheumatol., 21:1808-1813 (1994); Cuchacovich et al., Arthritis Rheum., 35:736-739 (1992)). The Sharp score is a very labor-intensive method (Van der Heijde, Baillieres Clin. Rheumatol., 10:435-533 (1996)). In late or destructive RA, the Sharp and the Larsen methods were found to provide similar information. However, the sensitivity to change of the various scoring methods late in the disease has not yet been investigated and it can be argued that the scoring methods that independently measure ERO and JSN provide useful information (Pincus et al., J. Rheumatol., 24:2106-2122 (1997)). See also Drossaers-Bakker et al., Arthritis Rheum., 43:1465-1472 (2000), which compared the three radiologic scoring systems for the long-term assessment of RA.

Paulus et al., Arthritis Rheum., 50:1083-1096 (2004) categorized radiographic joint damage as progressive or non-progressive in individuals with RA participating in clinical trials, and concluded that RA joint damage in an observational cohort can be classified as progressive or non-progressive with the use of a composite definition that includes a number of imprecise and related, but distinct, measures of structural joint damage. It appears that in day-to-day clinical management of an RA patient, an interval change between a pair of radiographs of at least five Sharp radiographic damage score units should be present before one considers the structural change to be real and uses it as the basis for a treatment decision.

Over the past 10 years there have been major advances in the treatment of RA. Combination use of existing disease-modifying anti-rheumatic drugs (DMARDs), together with new biologic agents, have provided higher levels of efficacy in a larger proportion of patients, while the early diagnosis and treatment of the disease has also improved outcomes.

Etanercept is a fully human fusion protein that inhibits TNF and the subsequent inflammatory cytokine cascade. Etanercept has been shown to be safe and effective in rapidly reducing disease activity in adults with RA and in sustaining that improvement (Bathon et al., N. Eng. J. Med., 343:1586-1593 (2000); Moreland et al., N. Engl. J. Med., 337:141-147 (1997); Moreland et al., Ann. Intern. Med., 130:478-486 (1999); Weinblatt et al., N. Engl. J. Med., 340:253-259 (1999); Moreland et al., J. Rheumatol., 28:1238-1244 (2001)). It is equally effective in children with polyarticular juvenile RA (Lovell et al., N. Engl. J. Med., 342:763-769 (2000)). Etanercept is approved for use as monotherapy, as well as combination therapy with MTX, for the treatment of RA. US 2007/0071747 discloses use of a TNF-alpha inhibitor for treatment of erosive polyarthritis.

Loss of function and radiographic change occur early in the course of the disease. These changes can be delayed or prevented with the use of certain DMARDs. Although several DMARDs are initially clinically effective and well tolerated, many of these drugs become less effective or exhibit increased toxicity over time. Based on its efficacy and tolerability, MTX has become the standard therapy by which other treatments are measured (Bathon et al., N. Eng. J. Med., 343:1586-1593 (2000); Albert et al., J. Rheumatol., 27:644-652 (2000)).

Recent studies have examined radiographic progression in patients with late-stage RA who have taken leflunomide, MTX, or placebo (Strand et al., Arch. Intern. Med., 159:2542-2550 (1999)) as well as patients who have taken infliximab plus MTX or placebo plus MTX following a partial response to MTX (Lipsky et al., N. Engl. J. Med., 343:1594-1602 (2000); Maini et al., Lancet, 354:1932-1939 (1999)). In the first year of the ENBREL™ ERA (early RA) trial, etanercept was shown to be significantly more effective than MTX in improving signs and symptoms of disease and in inhibiting radiographic progression (Bathon et al., N. Eng. J. Med., 343:1586-1593 (2000)). Genovese et al., Arthritis Rheum. 46:1443-1450 (2002) reports results from the second year of the study, concluding that etanercept as monotherapy was safe and superior to MTX in reducing disease activity, arresting structural damage, and decreasing disability over 2 years in patients with early, aggressive RA.

Further, reduction in radiographic progression in the hands and feet was observed in patients with early RA after receiving infliximab in combination with MTX (Van der Heijde et al., Annals Rheumatic Diseases 64:418-419 (2005)). Patients with early RA achieved a clinically meaningful and sustained improvement in physical function after treatment with infliximab (Smolen et al., Annals Rheumatic Diseases, 64:418 (2005)). The effect of infliximab and MTX on radiographic progression in patients with early RA is reported in Van der Heijde et al., Annals Rheumatic Diseases, 64:417 (2005). Infliximab treatment of patients with ankylosing spondylitis leads to changes in markers of inflammation and bone turnover associated with clinical efficacy (Visvanathan et al., Effects of infliximab on markers of inflammation and bone turnover and associations with bone mineral density in patients with ankylosing spondylitis, Ann Rheum Dis, February 2009; 68: 175-182.

The effect of infliximab therapy on bone mineral density in patients with ankylosing spondylitis (AS) resulting from a randomized, placebo-controlled trial named ASSERT is reported by Van der Heijde et al., Efficacy and safety of infliximab in patients with ankylosing spondylitis: results of a randomized, placebo-controlled trial (ASSERT). Arthritis Rheum 2005; 52:582-91. Infliximab was found to improve fatigue and pain in patients with AS, in results from ASSERT. Further, the efficacy and safety of infliximab in patients with AS as a result of ASSERT are described by van der Heijde et al., Arthritis Rheum., 52:582-591 (2005). The authors conclude that infliximab was well tolerated and effective in a large cohort of patients with AS during a 24-week study period. In addition, the effect of infliximab therapy on spinal inflammation was assessed by magnetic resonance imaging in a randomized, placebo-controlled trial of 279 patients with AS (Van der Heijde et al., Arthritis Rheum., 52:582-591 (2005). The manner in which the treatment effect on spinal radiographic progression in patients with AS should be measured is addressed by van der Heijde et al., Arthritis Rheum. 52(7):1979-1985 (2005).

The results of radiographic analyses of the infliximab multinational psoriatic arthritis controlled trial (IMPACT) after one year are reported by Antoni et al., The Infliximab Multinational Psoriatic Arthritis Controlled Trial (IMPACT): results of radiographic analyses after 1 year, Ann Rheum Dis, August 2006; 65: 1038-1043. Evidence of radiographic benefit of treatment with infliximab plus MTX in RA patients who had no clinical improvement, with a detailed subanalysis of data from the anti-TNF factor trial in RA with concomitant therapy study, is reported by Smolen et al., Arthritis Rheum. 52:1020-1030 (2005). Radiographic progression as measured by mean change in modified Sharp/van der Heijde score was much greater in patients receiving MTX plus placebo than in patients receiving infliximab plus MTX. The authors conclude that even in patients without clinical improvement, treatment with infliximab plus MTX provided significant benefit with regard to the destructive process, suggesting that in such patients these 2 measures of disease are dissociated. The association between baseline radiographic damage and improvement in physical function after treatment of patients having RA with infliximab is described by Breedveld et al., Annals Rheumatic Diseases, 64:52-55 (2005). Structural damage was assessed using the van der Heijde modification of the Sharp score. The authors conclude that greater joint damage at baseline was associated with poorer physical function at baseline and less improvement in physical function after treatment, underlining the importance of early intervention to slow the progression of joint destruction.

TNF was first identified as a serum-derived factor that was cytotoxic for several transformed cell lines in vitro and caused necrosis of certain tumors in vivo. A similar factor in the superfamily was identified and referred to as lymphotoxin (“LT”). Due to observed similarities between TNF and LT in the early 1980's, it was proposed that TNF and LT be referred to as TNF-α and TNF-β, respectively. Scientific literature thus makes reference to both nomenclatures. As used in the present application, the term “TNF” refers to TNF-α. Later research revealed two forms of LT, referred to as LTα and LTβ. US 2005-0129614 describes another polypeptide member of the TNF ligand super-family based on structural and biological similarities, designated TL-5.

Members of the TNF family of proteins exist in membrane-bound forms that act locally through cell-cell contact, or as secreted proteins. A family of TNF-related receptors react with these proteins and trigger a variety of signalling pathways including apoptosis, cell proliferation, tissue differentiation, and proinflammatory responses. TNF-α by itself has been implicated in inflammatory diseases; autoimmune diseases; viral, bacterial, and parasitic infections, malignancies, and neurodegenerative diseases and is a useful target for specific biological therapy in diseases such as RA and Crohn's disease.

Cloning of the TNF and LTα proteins and further characterization of their respective biological activities reveal that the proteins differ in many aspects. Aggarwal et al., Cytokines and Lipocortins in Inflammation and Differentiation, Wiley-Liss, Inc. 1990, pp. 375-384. For instance, LTα is a secreted, soluble protein of approximately 20 kDa (25 kDa if N- and O-glycosylated). TNF, in contrast, has no site for glycosylation and is synthesized with an apparent transmembrane domain that results in the original protein being cell associated. Proteolysis of the cell-associated TNF protein results in the release of the soluble form of the protein having a molecular weight of approximately 19 kDa. TNF is produced primarily by activated macrophages, whereas LT is produced by activated lymphocytes. Wong et al., Tumor Necrosis Factors: The Molecules and their Emerging Role in Medicine, Beutler, B., ed., Raven Press (1991), pp. 473-484. The sequences encoding TNF and LTα also differ. TNF and LTα share only approximately 32% amino acid sequence identity. Regarding the different biological activities of TNF and LTα, TNF increases production of endothelial-cell interleukin-1 (“IL-1”), whereas LTα has little effect thereon. Further, TNF induces production of macrophage-colony-stimulating factor from macrophages, whereas LTα has no effect thereon. These and other biological activities are discussed in Aggarwal, Tumor Necrosis Factors: Structure, Function and Mechanism of Action, Aggarwal and Vicek, eds. (1992), pp. 61-78.

TNF and LTα are described further in the review articles by Spriggs, “Tumor Necrosis Factor Basic Principles and Preclinical Studies,” Biologic Therapy of Cancer, DeVita et al., eds., J.B. Lippincott Company (1991) Ch. 16, pp. 354-377; Ruddle, Current Opinion in Immunology, 4:327-332 (1992); Wong et al., “Tumor Necrosis Factor,” Human Monocytes, Academic Press (1989), pp. 195-215; and Paul et al., Ann. Rev. Immunol., 6:407-438 (1988).

In non-tumor cells, TNF and TNF-related cytokines are active in a variety of immune responses. Both TNF and LTα ligands bind to and activate TNF receptors (p55 or p60 and p75 or p80; herein called “TNF-R”).

Cell-surface LT complexes have been characterized in CD4+ T cell hybridoma cells (II-23.D7), which express high levels of LT (Browning et al., J. Immunol., 147: 1230-1237 (1991); Androlewicz et al., J. Biol. Chem., 267: 2542-2547 (1992)). The expression and biological roles of LTβ-R, LT subunits, and surface LT complexes are reviewed in Ware et al., “The ligands and receptors of the lymphotoxin system”, in Pathways for Cytolysis, Current Topics Microbiol. Immunol., Springer-Verlag, pp. 175-218 (1995).

Lymphotoxin-α (LTα), which is also known as tumour necrosis factor-β (TNF-β), is produced after mitogenic stimulation by a variety of cells, including B cells. It lacks a transmembrane domain and is expressed on the cell surface as a heterotrimeric complex together with the transmembrane protein LT-β, a member of the TNF family. LT-αβ membrane complexes have been found on activated T, B and natural killer (NK) cells and differ in subunit composition, with the major form consisting of LT-α1β2. LT-α (TNF-β) is mitogenic for B cells and appears to play an important role in lymphocyte homing and formation of spleen and lymph nodes, as mice with disrupted LT-α (TNF-β) genes fail to develop peripheral lymph nodes and Peyer's patches.

LTα and LTβ are members of the TNF-SF. LTα expression is induced and LTα secreted primarily by activated T and B lymphocytes and natural killer (NK) cells. Among the T helper cell subclasses, LTα appears to be produced by Th1 but not Th2 cells. LTα has also been detected in melanocytes. LTβ (also called p33) has been identified on the surface of T lymphocytes, T cell lines, B cell lines and lymphokine-activated killer (LAK) cells. Studies have shown that LTβ is not functional in the absence of LTα.

LTα exists either as a homotrimer (LTα3) or a heterotrimer with LTβ. These heterotrimers contain either two subunits of LTα and one subunit of LTβ (LTα2β1), or one subunit of LTα and two of LTβ (LTα1β2). LTα is secreted from cells as the homotrimer (LTα3) or complexed on the cell surface with transmembrane LTβ predominantly as a LTα1β2 heterotrimer (Gramaglia I, et al., J Immunol 1999; 162(3):1333-8).

The two trimeric LT forms bind distinct receptors: LTα3 binds TNFRI and TNFRII; whereas LTα1β2 binds LTββR. The heterotrimeric form LTα2β1 likely binds TNF receptors. Signaling through the LTβR pathway is critical for the development of germinal center (GC) architecture and regulating normal development of secondary lymph nodes (LN) (Ware C F., Annu Rev Immunol 2005; 23:787-819). It has been implicated in the development of tertiary lymphoid structures in chronically-inflamed tissue associated with autoimmune disease (Weyland et al. J Rheumatol Suppl 2007; 79:9-14). Elevated LTα, LTβ and LTβR transcripts have been observed in synovial tissues of RA patients, and point to a role for the LT pathway in the pathogenesis of disease (Takemura et al., 2001, supra). Moreover, LTβ-R expression is increased in fibroblast-like synoviocytes in RA patients (Braun et al., Arthritis Rheum 2004; 50(7):2140-50).

LTβ-R has a well-described role both in the development of the immune system and in the functional maintenance of a number of cells in the immune system, including follicular dendritic cells and a number of stromal cell types (Matsumoto et al., Immunol. Rev. 156:137 (1997)). Known ligands to the LTβ-R include not only LTα1β2, but also a second ligand called LIGHT (Mauri et al., Immunity 8:21 (1998)). Activation of LTβ-R has been shown to induce the apoptotic death of certain cancer cell lines in vivo (U.S. Pat. No. 6,312,691). Humanized antibodies to LTβ-R and methods of use thereof are provided in US 2004-0058394 and stated as being useful for treating or reducing the advancement, severity, or effects of neoplasia in humans. Further, EP 1585547 (WO 2004/058183) (LePage and Gill) discloses combination therapies that include a composition that activates LTβ-R signaling in combination with one or more other chemotherapeutic agents, as well as therapeutic methods and screening methods for identifying agents that in combination with a LTβ-R agonist agent have an additive effect on tumor inhibition.

LT is important for lymphoneogenesis, as evident from knockout mice. See Futterer et al. Immunity, 9 (1): 59-70 (1998), showing that mice deficient in LTβ-R lacked lymph nodes and Peyer's patches and also showing impaired antibody affinity maturation. Rennert et al., Immunity, 9 (1): 71-9 (1998) reported that an agonist monoclonal antibody against LTβ-R restored the ability to make lymph nodes in LTα knockout mice. See also Wu et al., J. Immunology, 166 (3): 1684-9 (2001) and Endres et al., J. Exp. Med., 189 (1): 159-68 (1999); Dohi et al., J. Immunology, 167 (5): 2781-90 (2001); and Matsumoto et al., J. Immunology, 163 (3): 1584-91 (1999). Korner et al. Eur. J. Immun., 27 (10): 2600-9 (1997) reported that mice lacking both TNF and LT showed retarded B-cell maturation and serum immunoglobulin deficiencies, whereas mice lacking only TNF showed no such deficiencies.

In addition, LT is important for inflammation. LTα is overexpressed in the pancreas of RIP.LTα transgenic mice, which have shown inflammation, increased chemokine expression, and a lymphoid-like structure, and in which overexpression of LTβ alone has demonstrated no additional inflammation. Further, LTα-deficient mice exhibit impaired TNF-α production, and defective splenic architecture and function are restored when such mice are crossed to TNF-transgene (Kollias, J. Exp. Med., 188:745 (1998); Chaplin, Ann Rev Imm 17:399 (1999)), and decreased TNF levels are restored after pathogenic challenge (Eugster, Eur. J. Immun. 31:1935 (2001)).

When TNF-α or LTα₃ interacts with the TNF receptors TNFRI and/or TNFRII, the result is proinflammatory responses and/or apoptosis. When LTα1β2 interacts with the receptor LTβ-R, the result is lymphoneogenesis and induction of chemokines and adhesion molecules. Autoimmune diseases are associated with lymphoneogenesis and inflammatory responses, and there is increased LT expression in patients with autoimmune disease, including MS, inflammatory bowel disease (IBD), and RA (Weyand et al., Curr. Opin. Rheumatol., 15: 259-266 (2003); Selmaj et al., J. Clin. Invest., 87: 949-954 (1991); Matusevicius et al., J. Neuroimm., 66: 115-123 (1996); Powell et al., International Immunology, 2 (6): 539-44 (1990); Zipp et al., Annals of Neurology, 38/5: 723-730 (1995); Voskuhl et al., Autoimmunity 15 (2): 137-43 (1993); Selmaj et al., J. Immunology, 147: 1522-29 (1991); Agyekum et al., Journal Pathology, 199 (1): 115-21 (2003); and Takemura et al., J. Immunol., 167: 1072 (2001)).

As to RA specifically, levels of human LTα3 and TNF-α in RA patients are elevated over those of normal donors (Stepien, Eur Cytokine Net 9: 145 (1998)). The roles of LTα in RA include: serum LTα is present in some RA patients, increased LTα protein is present in synovium, the LT pathway is associated with ectopic lymphoneogenesis in synovium, and there is increased LTβ-R expression on fibroblast-like synoviocytes in RA patients. In addition, a case report discloses that neutralizing LTα3 is beneficial for an infliximab-resistant RA patient (Buch et al., Ann. Rheum. Dis., 63: 1344-46 (2004)). Also, Han et al., Arthrit. Rheumat., 52: 3202-3209 (2005) describes that blockading the LT pathway exacerbates autoimmune arthritis by enhancing the Th1 response.

Preclinical efficacy for prevention and treatment with LTβR-Fc in collagen-induced arthritis (CIA) is shown in Fava et al., J. Immunology, 171 (1): 115-26 (2003). Further, LTα-deficient mice are resistant to experimental autoimmune encephalomyelitis (EAE) (Suen et al., J. Exp. Med, 186: 1233-40 (1997); Sean Riminton et al., J. Exp. Med, 187 (9): 1517-28 (1998)). There is also published efficacy of LTβR-Fc in EAE (Gommerman et al., J. Clin. Invest, 112 (5): 755-67 (2003)). Also, LTβR-Fc disrupts lymphogenesis in mice. Mackay et al., Europ. J. Immunol. 27 (8): 2033-42 (1997)). Further, administration of LTβR-Fc decreases insulin-dependent diabetes mellitus (IDDM) in non-obese diabetic mice (Wu et al., J. Exp. Med, 193 (11): 1327-32 (2001)). The role of LT in lymphogenesis in non-human primates was investigated by Gommerman et al., J. Clin. Invest. 110 (9): 1359-69 (2002) using LTβR-Fc. Further, LTα-deficient mice are less susceptible to M. bovis BCG than TNF-α-deficient mice. Eugster et al., Europ. J. Immunol., 31: 1935 (2001).

Antagonists directed to interfere with the LT pathway have been identified as potential therapeutic agents for the treatment of autoimmune diseases. One such molecule in the pathway is lymphotoxin alpha (LTα), which is an attractive target because it has been shown to be capable of more interactions with various receptor in the pathway than other cytokines involved in the pathway, such as TNF-alpha or lymphotoxin beta (LTβ). LTα antagonistic antibodies have shown potential as therapeutic agents for the treatment of autoimmune diseases, such as rheumatoid arthritis (RA) (see Adams et al. WO/2008/06377, hereby incorporated by reference in its entirety). However, for any given RA arthritis patient one frequently cannot predict or prognosticate which patient is likely to respond to a particular treatment, even with newer LT antagonist therapies, thus necessitating considerable trial and error, often at considerable risk and discomfort to the patient, in order to find the most effective therapy.

Thus, there is a need for more effective means for determining which patients will respond to which treatment and for incorporating such determinations into more effective treatment regimens for RA patients with LT antagonist therapies, whether used as single agents or combined with other agents to treat RA.

The entire contents of all references cited herein are hereby incorporated by reference.

### SUMMARY OF THE INVENTION

The present invention provides soluble LTalpha-beta (solLTαβ) compositions and methods for use as a biomarker in the treatment autoimmune diseases, e.g. rheumatoid arthritis.

In one aspect, the present invention provides a method of assessing whether a patient with rheumatoid arthritis (RA) is responsive to treatment with a lymphotoxin (LT) antagonist, the method comprising assessing the amount of solLTαβ in the patient treated with the LT antagonist, wherein an increase in the amount of solLTαβ in the treated patient, as compared to the amount of solLTαβ in the untreated patient, indicates that the patient is responsive to treatment with the LT antagonist.

In another aspect, the present invention provides a method of monitoring the efficacy of treatment for rheumatoid arthritis (RA) in a patient, wherein the patient is treated with a LT antagonist, the method comprising monitoring the amount of solLTαβ in the patient treated with the LT antagonist, wherein an increase in the amount of solLTαβ in the treated patient, as compared to the amount of solLTαβ in the untreated patient, is indicative of the efficacy of the treatment with the LT antagonist.

In another aspect, the present invention provides a method of identifying a therapeutic agent effective to treat rheumatoid arthritis in a patient subpopulation, the method comprising correlating efficacy of the agent with the presence of an amount of solLTαβ in the patient subpopulation treated with the agent, wherein the amount of solLTαβ indicates that the patient subpopulation is responsive to the treatment with the agent, thereby identifying the agent as effective to treat rheumatoid arthritis in the patient subpopulation.

In another aspect, the present invention provides a method of predicting responsiveness of a patient, with rheumatoid arthritis, to treatment with a LT antagonist, comprising comparing the amount of solLTαβ in a sample obtained from the patient after treatment with the LT antagonist, to a sample obtained from the patient before the treatment, wherein an increased amount of the solLTαβ after treatment is indicative of responsiveness to treatment with the LT antagonist.

In another aspect, the present invention provides a method of monitoring responsiveness of a patient, with rheumatoid arthritis, to treatment with a LT antagonist, comprising comparing the amount of solLTαβ in a sample obtained from the patient after treatment with the LT antagonist, to a sample obtained from the patient before the treatment, wherein an increased amount of the solLTαβ after treatment is indicative of responsiveness to treatment with the LT antagonist.

In another aspect, the present invention provides a method of modifying a treatment of a patient with rheumatoid arthritis with a LT antagonist, comprising adjusting the amount of a LT antagonist administered to the patient based on a comparison of the amount of solLTαβ in the patient serum or synovial fluid before and after treatment with the LT antagonist, wherein an increased amount of solLTαβ is indicative of responsiveness to treatment with the LT antagonist.

In another aspect, the present invention provides a method of designing a treatment with a LT antagonist for a patient with rheumatoid arthritis, comprising determining the effective dosage of a LT antagonist administered to the patient based on a comparison of the amount of solLTαβ in the patient serum or synovial fluid before and after treatment with the LT antagonist, wherein the amount of solLTαβ is indicative of responsiveness to treatment with the LT antagonist.

In another aspect, the present invention provides a method of predicting prognosis of an autoimmune disease in a patient, comprising modifying the amount of a LT antagonist to be administered to the patient based on a comparison of the amount of solLTαβ in the patient serum or synovial fluid before and after treatment with the LT antagonist, wherein the amount of solLTαβ is indicative of the prognosis of the disease.

In another aspect, the present invention provides a method of monitoring responsiveness of patient with rheumatoid arthritis, to treatment with a LT antagonist, comprising comparing the amount of solLTαβ in a sample obtained from the patient after treatment with the LT antagonist, to a sample obtained from the patient before the treatment, wherein a sustained increased amount of the solLTαβ after treatment is indicative of responsiveness to treatment with the LT antagonist

In another aspect, the present invention provides a method of modifying a treatment of patient with rheumatoid arthritis with a LT antagonist, comprising adjusting the amount of a LT antagonist administered to the patient based on a comparison of the amount of solLTαβ in a sample obtained from the patient after treatment with the LT antagonist, to a sample obtained from the patient before the treatment, wherein an increased, and/or sustained increased, amount of the solLTαβ after treatment is indicative of responsiveness to treatment with the LT antagonist, and wherein the amount of LT antagonist is adjusted to obtain and/or sustain an increased amount of solLTαβ in the patient.

In some aspects of the above methods, the amount of solLTαβ can be in a range of 1-10,000 pg/mL in the patient serum. In one embodiment, the solLTαβ can be in a range of 25-800 pg/mL in the patient serum. In another embodiment, the amount of solLTαβ can be in the range of 20-400 pg/ml in the patient synovial fluid or tissue.

In another aspect, the present invention provides a method of diagnosing or predicting an autoimmune disease in a patient, comprising assessing the amount of solLTαβ in a sample obtained from the patient, wherein an amount of the solLTαβ is indicative of the disease. In one aspect, the patient is treated with a LT antagonist. In another aspect, the amount of solLTαβ is in the range of 10-500 pg/mL. In one aspect the sample is a serum sample.

In another aspect, the present invention provides a method of diagnosing or predicting a patient at risk for an autoimmune disease, comprising assessing the amount of solLTαβ in a sample obtained from the patient, wherein an amount of the solLTαβ is indicative of the disease. In one aspect, the patient is treated with a LT antagonist. In another aspect, the amount of solLTαβ is in the range of 10-500 pg/mL. In one aspect the sample is a serum sample.

In some aspects of the above methods, the amount of solLTαβ can be measured within 24 hours, 50 days or 100 days after receiving a first dose of the LT antagonist. In one embodiment, the antagonist can be an antibody or immunoadhesin (e.g., the antibody can be a chimeric, humanized, or human antibody). In another embodiment, the antibody can be an anti-lymphotoxin alpha (anti-LTα) antibody. In other embodiments, the antagonist is not conjugated with a cytotoxic agent or the antagonist can be conjugated with a cytotoxic agent.

In some embodiments, the LT antagonist can be administered intravenously or the LT antagonist can be administered subcutaneously. In another, the LT antagonist can be administered into an affected joint.

In some embodiments, the patient may have never been previously administered a medicament for the rheumatoid arthritis, the patient may have been previously administered at least one medicament for the rheumatoid arthritis, or the patient may not be responsive to the at least one medicament that was previously administered. In another embodiment, the previously administered medicament or medicaments can be an immunosuppressive agent, cytokine antagonist, integrin antagonist, corticosteroid, analgesic, a disease-modifying anti-rheumatic drug (DMARD), or a non-steroidal anti-inflammatory drug (NSAID).

In one embodiment, the LT antagonist treatment can further comprise administering an effective amount of one or more second medicaments with the LT antagonist, wherein the LT antagonist is a first medicament. In other embodiments, the second medicament can be more than one medicament. In other embodiments, the second medicament can be an immunosuppressive agent, a DMARD, a pain-control agent, an integrin antagonist, a NSAID, a cytokine antagonist, a bisphosphonate, or a combination thereof.

In one embodiment, the immunosuppressive agent can be selected from the group consisting of etanercept, infliximab, adalimumab, leflunomide, anakinra, azathioprine, and cyclophosphamide;

In one embodiment, the second medicament is a DMARD selected from the group consisting of auranofin, chloroquine, D-penicillamine, injectable gold, oral gold, hydroxychloroquine, sulfasalazine, myocrisin and methotrexate. In one embodiment, the second medicament is a NSAID selected from the group consisting of fenbufen, naprosyn, diclofenac, etodolac, indomethacin, aspirin and ibuprofen.

In one embodiment, the second medicament is a corticosteroid selected from the group consisting of prednisone, prednisolone, methylprednisolone, hydrocortisone, or dexamethasone.

In one embodiment, the second medicament can be selected from the group consisting of anti-alpha4, etanercept, infliximab, etanercept, adalimumab, kinaret, efalizumab, osteoprotegerin (OPG), anti-receptor activator of NFκB ligand (anti-RANKL), anti-receptor activator of NFκB-Fc (RANK-Fc), pamidronate, alendronate, actonel, zolendronate, clodronate, methotrexate, azulfidine, hydroxychloroquine, doxycycline, leflunomide, sulfasalazine (SSZ), prednisolone, interleukin-1 receptor antagonist, prednisone, and methylprednisolone.

In another embodiment, the second medicament can be selected from the group consisting of infliximab, an infliximab/methotrexate (MTX) combination, MTX, etanercept, a corticosteroid, cyclosporin A, azathioprine, auranofin, hydroxychloroquine (HCQ), combination of prednisolone, MTX, and SSZ, combinations of MTX, SSZ, and HCQ, the combination of cyclophosphamide, azathioprine, and HCQ, and the combination of adalimumab with MTX.

In one other embodiment, the second medicament can be MTX. In another embodiment, the MTX can be administered perorally or parenterally.

In one embodiment, the methods pertain to a patient having rheumatoid arthritis (RA). In another embodiment, the RA can be early rheumatoid arthritis or incipient rheumatoid arthritis. In other embodiments, the patient can have exhibited an inadequate response to one or more anti-tumor necrosis factor (anti-TNF) inhibitors.

In one embodiment, the amount of the solLTαβ is measured within 24 hours, 50 days or 100 days after receiving a first dose of the LT antagonist.

In another embodiment, the previously administered medicament(s) can be administered at least about three months before the LT antagonist treatment. In another embodiment, the LT antagonist can be administered without any other medicament to treat the RA.

In one embodiment, the method of monitoring responsiveness of an RA patient to treatment with a LT antagonist comprises the use of a test. In one embodiment, the test is an imaging test that measures a reduction in bone or soft tissue joint damage as compared to a baseline prior to the treatment. In another embodiment, the test can measure a total modified Sharp score.

In one other embodiment, the amount of the LT antagonist administered is effective in achieving a reduction in the joint damage.

In another embodiment, the method can further comprise re-treating the patient by administering an effective amount of the LT antagonist to the patient. In other embodiments, the re-treatment is commenced at least about 24 weeks after the first administration of the antagonist. In yet another embodiment, the amount of the LT antagonist administered upon each administration thereof can be effective to achieve a continued or maintained reduction in joint damage. In other embodiments, the method can comprise a further re-treatment commenced with an effective amount of the LT antagonist. In another embodiment, the further re-treatment can be commenced at least about 24 weeks after the second administration of the antagonist. In one embodiment, the joint damage can have been reduced after the re-treatment. In another embodiment, no clinical improvement can be observed in the patient at the time of the testing after the re-treatment.

In another aspect, the present invention provides a method of treating rheumatoid arthritis in a patient comprising first administering an effective amount of a LT antagonist to the patient to treat the rheumatoid arthritis, provided that a sample from the patient contains an amount of a LT (e.g., solLTαβ and LTα3) that is greater than the amount of LT in a control wherein the greater amount is indicative of responsiveness of the patient to the LT antagonist treatment and at least about 24 weeks after the first administration of the LT antagonist re-treating the patient by administering an effective amount of the LT antagonist to the patient, wherein no clinical improvement is observed in the patient at the time of the testing after the first administration of the LT antagonist.

In one aspect, the test sample is serum, synovial tissue or synovial fluid. In one aspect the control sample is a synovial fluid sample from an osteoarthritis patient's affected joint or from the RA patient's affected joint prior to treatment. In another aspect the control sample is from a normal donor serum sample or a pre-treatment sample from the RA patient.

In one embodiment, the testing is implemented using an apparatus adapted to determine the level of solLTαβ. In another embodiment, the testing is performed by using a software program executed by a suitable processor. In certain embodiments, the program is embodied in software stored on a tangible medium. In certain other embodiments, the tangible medium is selected from the group consisting of a CD-ROM, a floppy disk, a hard drive, a DVD, and a memory associated with the processor.

In certain embodiments, the methods of the invention further include a step of preparing a report recording the results of the testing or the diagnosis. In one embodiment, the report is recorded or stored on a tangible medium. In a specific embodiment, the tangible medium is paper. In another embodiment, the tangible medium is selected from the group consisting of a CD-ROM, a floppy disk, a hard drive, a DVD, and a memory associated with the processor.

In certain other embodiments, the methods of the invention further include a step of communicating the results of the diagnosis to an interested party. In one embodiment, the interested party is the patient or the attending physician. In another embodiment, the communication is in writing, by email, or by telephone.

### BRIEF DESCRIPTION OF THE DRAWINGS

FIG. 1A shows a schematic for a specific electrochemiluminescent assay (ECLA) for human LTαβ heterotrimers. FIG. 1B shows the specificity of this assay for detecting only human LTαβ and not other TNF family ligands. The assay using huLTβR-Fc capture and anti-LTα detection specifically recognizes LTα1β2 and LTα2β1; but not LTα3, TNFα or LIGHT. In FIG. 1C 293 cells stably transfected with LTα and LTβ constructs were stained with huLTβR-Fc-Alexa-647 and analyzed for surface LTαβ expression by FACS (open histogram); untransfected cells or cells stained with a control antibody are also shown. In FIG. 1D culture supernatants from untransfected 293 cells and cells transfected with LTα and LTβ constructs were analyzed using specific LTα3, LTαβ, and TNFα assays to measure levels of soluble cytokines (bars show average and SD of 4 cultures). The lowest detection limit for each assay is indicated with dashed line.

FIG. 2 illustrates that activated human T cells shed LTαβ by ADAM17 protease cleavage. (A) Culture supernatants from polarized human T cells 2 days post-reactivation were analyzed using specific LTα3, LTαβ, and TNFα assays to measure levels of soluble cytokines (bar graphs show average and SD of 3 blood donors). (B) Culture supernatants from Th1 human T cells treated −/+10 or 50 μM TNFα protease inhibitor-1 (TAPI-1) for 1 day post reactivation were analyzed as in panel A for levels of soluble cytokines (bars show average and SD of 3 blood donors). (C) RNA was isolated from the cell populations in panel B and quantified by PCR using LTα, LTβ and TNFα specific DNA probes. (D) Supernatant from pooled polarized Th1 cells was immunoprecipitated with anti-LTα-conjugated or LTβR-Fc-conjugated agarose beads, denatured proteins separated by gel electrophoresis, and Western blotted using fluorescent dye labeled probes specific for LTα (red) and LTβ (green). Recombinant human LTα1β2 was used as a reference. Two glycosylated forms each are seen for LTα and LT\tilde{β}

FIG. 3 shows elevated solLTαβ levels in serum of experimental autoimmune encephalomyelitis (EAE) mice dosed with muLTβR-Fc.

FIG. 4 shows (A) elevated solLTαβ levels in serum of collagen induced arthritis (CIA) mice dosed with muLTβR-Fc, and (B) elevated soluble TNF-α levels in serum of CIA mice dosed with TNFRII-Fc.

FIG. 5 shows levels of soluble human LTαβ in serum of human SCID mice (transplanted with human peripheral blood mononuclear cells) which developed severe graft versus host disease. Levels of soluble human LTαβ were elevated in mice treated with control antibody (Herceptin) but greatly reduced in mice treated with CTLA-4-Fc (anti-inflammatory therapeutic).

FIG. 6 shows peripheral solLTαβ in serum and synovial fluid of RA patients. (A) Sera collected from normal human donors and RA patients were analyzed using specific LTα3, LTαβ, and TNFα assays for levels of soluble cytokines (horizontal lines depict averages). (B) Synovial fluid collected from swollen joints of RA and OA patients was analyzed using specific LTα3, LTαβ, and TNFα assays for levels of soluble cytokines (horizontal lines depict averages).

FIG. 7 shows soluble LTαβ and LTα3 induce the expression of proinflammatory cytokines, chemokines and adhesion molecules in primary RA fibroblast-like synoviocytes (FLS). (A) Primary RA FLS lines were simulated with 300 ng/mL LTαβ or media alone for 6 h. Total RNA was purified from the cells and quantitative PCR performed for the genes shown. (B) FLS were simulated with 100 ng/mL LTα3 or media alone for 6 h. Total RNA was purified from the cells and quantitative PCR performed for the genes shown. Data are shown as mean±SEM and all differences between control and cytokines were highly significant by paired t test (p values<0.04). (C) FLS were stimulated with LTαβ or LTα3 alone or in the presence of 25 μg/mL LTβR-Fc or TNFRII-Fc. Total RNA was purified from the cells and quantitative PCR performed for the genes shown.

### DETAILED DESCRIPTION

### I. Abbreviations

The following abbreviations apply unless indicated otherwise:

| Abbreviation | Definition |
|---|---|
| LT | Lymphotoxin |
| LTa or LTα or LTalpha | Lymphotoxin-alpha |
| LTb or LTβ or LTbeta | Lymphotoxin-beta |
| LTab or LTαβ or LTalpha-beta | Lymphotoxin alpha-beta |
| soluble LTalpha-beta or solLTab | Soluble Lymphotoxin alpha-beta |
| or solLTαβ or solLTab | |
| huLTαβ | Human Lymphotoxin alpha-beta |
| OA | Osteoarthritis |
| RA | Rheumatoid arthritis |
| LTβR | Lymphotoxin-beta receptor |
| LTβR-Fc or LTβR-Ig | Lymphotoxin-beta receptor conjugated |
| | to an immunoglobulin Fc region |
| TNF | Tumor Necrosis Factor |
| TNFR | Tumor Necrosis Factor receptor |
| TNFR-Fc or TNFR-Ig | Tumor Necrosis Factor receptor |
| | conjugated to an immunoglobulin |
| | Fc region |
| FLS | Fibroblast-like Synoviocytes |
| PAb | Polyclonal antibodies |
| IL | Interleukin |
| DMEM | Dulbecco's Modified Eagle Medium |
| CXCL1 (GROα) | Chemokine (C-X-C motif) ligand 1 |
| | previously called GRO1 oncogene, |
| | GROα, KC, Neutrophil-activating |
| | protein 3 (NAP-3) and melanoma |
| | growth stimulating activity, alpha |
| | (MSGA-α). |
| CXCL2 (CROβ) | Chemokine (C-X-C motif) ligand 2 also |
| | called macrophage inflammatory protein |
| | 2-alpha (MIP2α), Growth-regulated |
| | protein beta (Groβ) and Gro oncogene- |
| | 2 (Gro-2). |
| VCAM-1 | Vascular cell adhesion molecule 1 also |
| | known as CD106 |
| ICAM-1 | Inter-Cellular Adhesion Molecule 1 also |
| | known as CD54 |
| RPL19 | Ribosomal protein L19 |
| ECLA | Electrochemiluminescent Assay |

### II. Definitions

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction, (Mullis et al., ed., 1994); A Practical Guide to Molecular Cloning (Perbal Bernard V., 1988); Phage Display: A Laboratory Manual (Barbas et al., 2001).

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

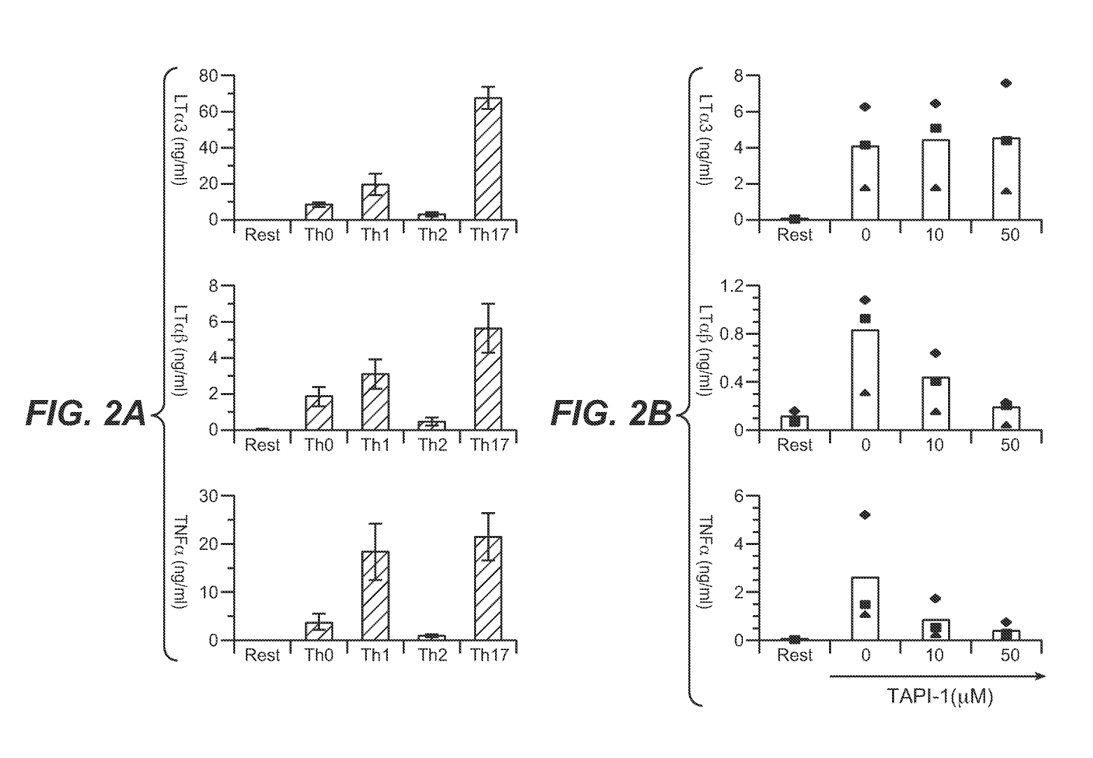

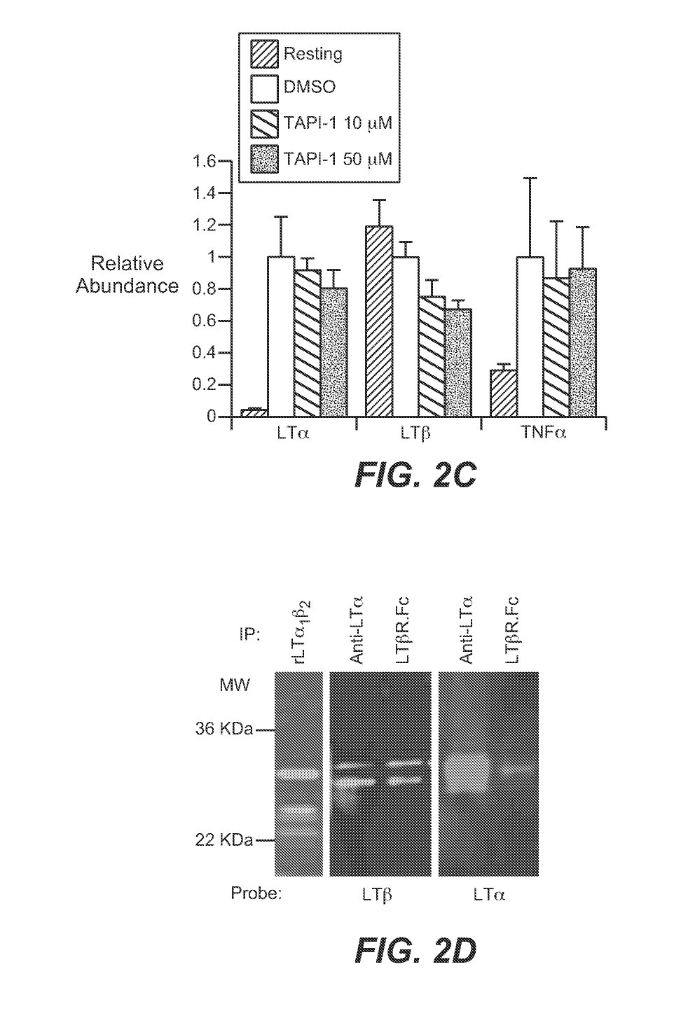

“Lymphotoxin-alpha” or “LTα” or “LTa” is defined herein as a monomeric protein having a relative molecular mass of 25,000. The protein has the sequence shown in FIG. 2A of U.S. Pat. No. 5,824,509 (and identified herein as SEQ ID NO:1) or the leu+1 (also called the leucyl amino-terminal lymphotoxin species) or his+24 (also called the histidyl amino-terminal lymphotoxin species) as disclosed in U.S. Pat. No. 5,824,509.

(SEQ ID NO: 1)
MTPPERLFLPRVCGTTLHLLLLGLLLVLLPGAQGLPGVGLTPSAAQTAR
QHPKMHLAHSTLKPAAHLIGDPSKQNSLLWRANTDRAFLQDGFSLSNNS
LLVPTSGIYFVYSQVVFSGKAYSPKATSSPLYLAHEVQLFSSQYPFHVP
LLSSQKMVYPGLQEPWLHSMYHGAAFQLTQGDQLSTHTDGIPHLVLSPS
TVFFGAFAL

Specifically, LTα is a member of the TNF superfamily and is secreted from cells as the homotrimer LTα3 (defined below), or complexed on the cell surface together with LTβ (defined below) as LTαβ (defined below), predominantly as the LTα1β2 heterotrimer. LTα is defined to specifically exclude human TNF-α or its natural animal analogues (Pennica et al., Nature 312:20/27: 724-729 (1984) and Aggarwal et al., J. Biol. Chem. 260: 2345-2354 (1985)). LTα is defined to specifically exclude human LTβ as defined, for example, in U.S. Pat. No. 5,661,004.

“Lymphotoxin-beta” or “LTβ” or “LTb” is defined herein as a biologically active polypeptide having the amino acid sequence shown as SEQ ID NO:2 in U.S. Pat. No. 5,661,004. LTβ is defined to specifically exclude human LTα as defined, for example, in U.S. Pat. No. 5,824,509.

“Lymphotoxin-alpha3” or “Lymphotoxin-α3 trimer” or “LTα3” or “LTa3” refers to a homotrimer of LTα monomers. It is a glycoprotein with a relative molecular mass (Mr) of 55,000-70,000 and is formed by the association of three LTα monomers.

“Lymphotoxin-alpha-beta” or “Lymphotoxin-αβ” or “LTαβ” or “LTαβ complex” or “LTab” refers to a membrane bound heterotrimer of LTα with LTβ. These heterotrimers contain either two subunits of LTα and one subunit of LTβ (LTα2β1), or one subunit of LTa and two of LTβ (LTα1β2). The term encompasses LTα2β1 or LTα1β2, individually, or a mixture thereof.

The term “soluble Lymphotoxin-alpha-beta” or “solLTαβ” refers to a LTαβ in solution, not associated or bound to a cell. The solLTαβ are defined by the LTβ having been cleaved at any point between the end of the transmembrane region (i.e., at about amino acid 44 of SEQ ID NO:2 in U.S. Pat. No. 5,661,004) and about amino acid 95.

“Tumor necrosis factor receptor-I” or “TNFRI” and “tumor necrosis factor receptor-II” or “TNFRII” refer to cell-surface TNF receptors for the LTα3 homotrimer, also known as p55 and p75, respectively.

“Lymphotoxin-beta receptor” or “Lymphotoxin-β receptor” or “LTβ-R” or “LTb” refers to the receptor to which the LTαβ heterotrimers bind. As used herein, the term “a lymphotoxin receptor” refers to the lymphotoxin-β receptor.

“Regulatory cytokines” are cytokines the abnormal levels of which indicate the presence of an autoimmune disorder in a patient. Such cytokines include, for example, interleukin-1 (IL-1), IL-2, IL-3, IL-4, IL-5, IL-6, IL-7, IL-8, IL-10, IL-12, IL-13, IL-14, IL-15, IL-18, IL-23, IL-24, IL-25, IL-26, BLyS/April, TGF-α, TGF-β, interferon-α (IFN-α), IFN-β, IFN-γ, MIP-1, MIF, MCP-1, M-CSF or G-CSF, a lymphotoxin, LIGHT, 4-1BB ligand, CD27 ligand, CD30 ligand, CD40 ligand, Fas ligand, GITR ligand, OX40 ligand, RANK ligand, THANK, TRAIL, TWEAK and VEG1. This group includes TNF family members, which include but are not limited to, TNF-α, lymphotoxins (LTs) such as LTα, LTβ, and LIGHT. For a review of the TNF superfamily, see MacEwan, Br. J. Pharmacology 135: 855-875 (2002). Preferably, the regulatory cytokine is a lymphotoxin such as a TNF family member.

“Inflammatory cytokines associated with rheumatoid arthritis” refer to lymphotoxins, such as LTα, associated with RA pathology, which can be inhibited systemically and/or in the joints or in an in vitro collagen-induced arthritis assay.

“LTαβ-expressing cells” are cells that express and/or shed the LTαβ heterotrimers.

The expression “modulates LTαβ-expressing cells” refers to depleting or altering proteins made by the cells such as cytokines, chemokines, or growth factors, with the cells including, for example, monocytes, dendritic cells, T cells, and B cells.

A “lymphotoxin antagonist” or “LT antagonist” is a molecule that reduces or prevents the binding of a LT to its corresponding lymphotoxin receptor (LTR) in a mammal and/or interferes with one or more LTR expressing cell functions, e.g., by reducing or preventing a proinflammatory response elicited by the LTR-expressing cell. The LT antagonist can decrease, block, inhibit, abrogate, modulate and/or otherwise interfere with LT activity in vitro, in situ, and/or in vivo. Such an agent can inhibit a biological function or activity of a LT, e.g., through binding to a LT and neutralizing its activity. For example, a LT antagonist can decrease block, abrogate, modulate, interfere, prevent and/or inhibit lymphotoxin RNA, DNA, or protein synthesis, lymphotoxin release, lymphotoxin receptor signaling, membrane lymphotoxin cleavage, lymphotoxin activity, and lymphotoxin production and/or synthesis. Examples of LT antagonists include, but are not limited to, anti-LT antibodies, antigen-binding fragments thereof, specified mutants or domains thereof that bind specifically to a LT that, upon binding to a LT, interfere with one or more functions of cells expressing a receptor for the LT, a soluble lymphotoxin receptor or fragment, fusion polypeptides thereof, a small-molecule LT antagonist, e.g., TNF binding protein I or II (TBP-I or TBP-II), nerelimonmab, CDP-571, infliximab (REMICADE®), etanercept (ENBREL®), adalimulab (HUMIRA™), CDP-571, CDP-870, afelimomab, lenercept, and the like), antigen-binding fragments thereof, and receptor molecules that bind specifically to a LT, compounds that prevent and/or inhibit lymphotoxin synthesis, LT release, or its action on target cells, such as thalidomide, tenidap, phosphodiesterase inhibitors (e.g, pentoxifylline and rolipram), A2b adenosine receptor agonists, and A2b adenosine receptor enhancers, compounds that prevent and/or inhibit lymphotoxin receptor signaling, such as mitogen-activated protein (MAP) kinase inhibitors, compounds that block and/or inhibit membrane lymphotoxin cleavage, such as metalloproteinase inhibitors, compounds that block and/or inhibit lymphotoxin activity, such as angiotensin-converting enzyme (ACE) inhibitors (e.g., captopril), and compounds that block and/or inhibit lymphotoxin production and/or synthesis, such as MAP kinase inhibitors. A preferred antagonist comprises an antibody or an immunoadhesin. Examples of LT antagonists contemplated herein are etanercept (ENBREL®), infliximab (REMICADE®), and adalimumab (HUMIRA™). In one embodiment, the LT antagonist is an antagonist of LTα, e.g., an anti-LTα antibody, and more particularly a humanized, monoclonal anti-LTα antibody.

As used herein, “antagonist” includes any molecule that partially or fully blocks, inhibits, or neutralizes a biological activity of a native polypeptide disclosed herein. Suitable antagonist molecules specifically include antagonist antibodies or antibody fragments, fragments or amino acid sequence variants of native polypeptides, peptides, antisense oligonucleotides, and small organic molecules, as non limiting examples. Methods for identifying antagonists may comprise contacting such a polypeptide, including a cell expressing it, with a candidate agonist or antagonist molecule and measuring a detectable change in one or more biological activities normally associated with such polypeptide.

A “blocking” antibody or an “antagonist” antibody is one that inhibits or reduces biological activity of the antigen it binds. Preferred blocking antibodies or antagonist antibodies substantially or completely inhibit the biological activity of the antigen.

An “anti-lymphotoxin antibody antagonist” or “anti-LT antibody antagonist” as used herein is an antibody that is a LT antagonist. For example, in some embodiments the anti-lymphotoxin antibody antagonist reduces or prevents the binding of a LT to its corresponding lymphotoxin receptor in a mammal and/or interferes with one or more lymphotoxin receptors (LTR) expressing cell functions, e.g. by reducing or preventing a proinflammatory response elicited by the LTR-expressing cell. The antibody that binds a LT may be designated as follows: an antibody that binds to lymphotoxin alpha (LTα), an “anti-lymphotoxin alpha antibody”, or an “anti-LTα antibody”. Antagonists can be screened by various methods known in the art for anti-inflammatory effects. For example, a method of screening can be employed as described in the following references: Lu, Y., et al., Current Opinion in Pharmacology 7, 571-546 (2007); Han, S., et al. Arthritis Rheum 52, 3202-3209 (2005); Fava, R. A., et al., J Immunol 171, 115-126 (2003); Mackay, F., et al., Gastroenterology 115, 1464-1475 (1998); Gommerman, J. L., et al., Nat Rev Immunol 2003 supra); Wu, Q., et al., J Exp Med 193, 1327-1332 (2001); and Ettinger, R., et al., J Exp Med 193, 1333-1340 (2001).

The term “modulate” as used herein refers to up or down regulation or change e.g., in an activity or function of a biological molecule. For example, modulate can refer to the change or up or down regulation of expression of a gene, level of RNA molecule or equivalent RNA molecules encoding one or more proteins or protein subunits, or activity of one or more proteins or protein subunits, such that expression, level, or activity is greater than or less than that observed in the absence of the modulator. For example, a LT antagonist may act as a modulator upon the activity of a LT polypeptide and/or a lymphotoxin receptor polypeptide.

The terms “antibody” and “immunoglobulin” are used interchangeably in the broadest sense and include monoclonal antibodies (e.g., full-length or intact monoclonal antibodies), polyclonal antibodies, multivalent antibodies, and multispecific antibodies (e.g., bispecific antibodies so long as they exhibit the desired biological activity), and may also include certain antibody fragments (as described in greater detail herein). An antibody can be chimeric, human, humanized, and/or affinity matured.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy-chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W. B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

“Antibody fragments” comprise a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody-hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains that enables the scFv to form the desired structure for antigen binding. For a review of scFv, see, e.g., Pluckthün, in The Pharmacology of Monoclonal Antibodies, vol. 113, Rosenburg and Moore eds. (Springer-Verlag, New York: 1994), pp 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med., 9:129-134 (2003); and Hollinger et al., Proc. Natl. Acad. Sci. USA, 90:6444-6448 (1993). Triabodies and tetrabodies are also described in Hudson et al., Nat. Med., 9:129-134 (2003).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14(3):253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas, 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol., 222:581-597 (1992); Sidhu et al., J. Mol. Biol., 338(2):299-310 (2004); Lee et al., J. Mol. Biol., 340(5):1073-1093 (2004); Fellouse, Proc. Natl. Acad. Sci. USA, 101(34):12467-12472 (2004); and Lee et al., J. Immunol. Methods, 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90: 2551 (1993); Jakobovits et al., Nature, 362: 255-258 (1993); Bruggemann et al., Year in Immunol., 7:33 (1993); U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368:856-859 (1994); Morrison, Nature, 368:812-813 (1994); Fishwild et al., Nature Biotechnol., 14:845-851 (1996); Neuberger, Nature Biotechnol., 14:826 (1996); and Lonberg and Huszar, Intern. Rev. Immunol., 13:65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g., U.S. Pat. No. 4,816,567 and Morrison et al., Proc. Natl. Acad. Sci. USA, 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all, or substantially all, of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature, 321:522-525 (1986); Riechmann et al., Nature, 332:323-329 (1988); and Presta, Curr. Op. Struct. Biol., 2:593-596 (1992). See also, for example, Vaswani and Hamilton, Ann. Allergy, Asthma & Immunol., 1:105-115 (1998); Harris, Biochem. Soc. Transactions, 23:1035-1038 (1995); Hurle and Gross, Curr. Op. Biotech., 5:428-433 (1994); and U.S. Pat. Nos. 6,982,321 and 7,087,409.

A “human antibody” is one which possesses an amino-acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain which are hypervariable in sequence and/or form structurally defined loops. Generally, antibodies comprise six HVRs; three in the VH(H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity, 13:37-45 (2000); Johnson and Wu in Methods in Molecular Biology, 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature, 363:446-448 (1993) and Sheriff et al., Nature Struct. Biol., 3:733-736 (1996).

A number of HVR delineations are in use and are encompassed herein. The HVRs that are Kabat complementarity-determining regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol., 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat Numbering) |
| H1 | H31-H35 | H26-H35 | H26-H32 | H30-H35 |
| | | | | (Chothia Numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

An “affinity-matured” antibody is one with one or more alterations in one or more HVRs thereof which result in an improvement in the affinity of the antibody for antigen, compared to a parent antibody which does not possess those alteration(s). In one embodiment, an affinity-matured antibody has nanomolar or even picomolar affinities for the target antigen. Affinity-matured antibodies are produced by procedures known in the art. For example, Marks et al., Bio/Technology, 10:779-783 (1992) describes affinity maturation by VH- and VL-domain shuffling. Random mutagenesis of HVR and/or framework residues is described by, for example: Barbas et al., Proc Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154(7):3310-3319 (1995); and Hawkins et al., J. Mol. Biol., 226:889-896 (1992).

“Growth-inhibitory” antibodies are those that prevent or reduce proliferation of a cell expressing an antigen to which the antibody binds. For example, the antibody may prevent or reduce proliferation of B cells in vitro and/or in vivo.

Antibodies that “induce apoptosis” are those that induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies). Antibody “effector functions” refer to those biological activities attributable to the Fc region (a native-sequence Fc region or amino-acid-sequence-variant Fc region) of an antibody, and vary with the antibody isotype. Examples of antibody effector functions include: C1q binding and complement-dependent cytotoxicity (CDC); Fc-receptor binding; antibody-dependent cell-mediated cytotoxicity (ADCC); phagocytosis; down-regulation of cell-surface receptors (e.g. B-cell receptor); and B-cell activation.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise herein, the numbering of the residues in an immunoglobulin heavy chain is that of the EU index as in Kabat et al., supra. The “EU index as in Kabat” refers to the residue numbering of the human IgG1 EU antibody.

A “functional Fc region” possesses an “effector function” of a native-sequence Fc region. Exemplary “effector functions” include C1q binding; CDC; Fc-receptor binding; ADCC; phagocytosis; down-regulation of cell-surface receptors (e.g. B-cell receptor; BCR), etc. Such effector functions generally require the Fc region to be combined with a binding domain (e.g. an antibody-variable domain) and can be assessed using various assays as disclosed, for example, in definitions herein.

A “native-sequence Fc region” comprises an amino acid sequence identical to the amino acid sequence of an Fc region found in nature. Native-sequence human Fc regions include a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence which differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g. from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering the nucleic acid encoding the antibody. Accordingly, a composition comprising an antibody having an Fc region according to this invention can comprise an antibody with K447, with all K447 removed, or a mixture of antibodies with and without the K447 residue.

“Fc receptor” or “FcR” describes a receptor that binds to the Fc region of an antibody. In some embodiments, an FcR is a native-human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward, Immunology Today, 18 (12):592-8 (1997); Ghetie et al., Nature Biotechnology, 15 (7):637-40 (1997); Hinton et al., J. Biol. Chem., 279(8):6213-6 (2004); WO 2004/92219 (Hinton et al.).

Binding to human FcRn in vivo and serum half-life of human FcRn high-affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See, also, for example, Shields et al., J. Biol. Chem., 9(2): 6591-6604 (2001).

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural-killer (NK) cells, monocytes, cytotoxic T cells, and neutrophils. The effector cells may be isolated from a native source, e.g., from blood.

“Antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enables these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol., 9:457-492 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA), 95:652-656 (1998).

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g. as described in Gazzano-Santoro et al., J. Immunol. Methods, 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 and WO 1999/51642. See, also, e.g., Idusogie et al., J. Immunol. 164:4178-4184 (2000).

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

In one embodiment, the “Kd” or “Kd value” according to this invention is measured by a radiolabeled antigen-binding assay (RIA) performed with the Fab version of an antibody of interest and its antigen as described by the following assay. Solution-binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol., 293:865-881 (1999)). To establish conditions for the assay, microtiter plates (DYNEX Technologies, Inc.) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen antigen are mixed with serial dilutions of a Fab of interest (e.g., consistent with assessment of the anti-VEGF antibody, Fab-12, in Presta et al., Cancer Res., 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% TWEEN-20™ surfactant in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, the Kd or Kd value is measured by using surface-plasmon resonance assays using a BIACORE®-2000 or a BIACORE®-3000 instrument (BIAcore, Inc., Piscataway, N.J.) at 25° C. with immobilized antigen CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately ten response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% TWEEN20™ surfactant (PBST) at 25° C. at a flow rate of approximately 25 μl/min. Association rates (kₒₙ) and dissociation rates (k_{off}) are calculated using a simple one-to-one Langmuir binding model (BIAcore® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol., 293:865-881 (1999). If the on-rate exceeds 10⁶ M⁻¹s⁻¹ by the surface-plasmon resonance assay above, then the on-rate can be determined by using a fluorescent quenching technique that measures the increase or decrease in fluorescence-emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow-equipped spectrophotometer (Aviv Instruments) or a 8000-series SLM-AMINCO™ spectrophotometer (ThermoSpectronic) with a stirred cuvette.

An “on-rate,” “rate of association,” “association rate,” or “kₒₙ” according to this invention can also be determined as described above using a BIACORE®-2000 or a BIACORE®-3000 system (BIAcore, Inc., Piscataway, N.J.).

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

In certain embodiments, the humanized antibody useful herein further comprises amino acid alterations in the IgG Fc and exhibits increased binding affinity for human FcRn over an antibody having wild-type IgG Fc, by at least 60 fold, at least 70 fold, at least 80 fold, more preferably at least 100 fold, preferably at least 125 fold, even more preferably at least 150 fold to about 170 fold.

The N-glycosylation site in IgG is at Asn297 in the CH2 domain. Included for use in therapy herein are compositions of any humanized antibodies having an Fc region, wherein about 80-100% (and preferably about 90-99%) of the antibody in the composition comprises a mature core carbohydrate structure that lacks fucose, attached to the Fc region of the glycoprotein, or has reduced fucose content.

As used herein, “rheumatoid arthritis” or “RA” refers to a recognized disease state that may be diagnosed according to the 2000 revised American Rheumatoid Association criteria for the classification of RA, or any similar criteria. The term includes not only active and early RA, but also incipient RA, as defined below. Physiological indicators of RA include, symmetric joint swelling which is characteristic though not invariable in RA. Fusiform swelling of the proximal interphalangeal (PIP) joints of the hands as well as metacarpophalangeal (MCP), wrists, elbows, knees, ankles, and metatarsophalangeal (MTP) joints are commonly affected and swelling is easily detected. Pain on passive motion is the most sensitive test for joint inflammation, and inflammation and structural deformity often limits the range of motion for the affected joint. Typical visible changes include ulnar deviation of the fingers at the MCP joints, hyperextension, or hyperflexion of the MCP and PIP joints, flexion contractures of the elbows, and subluxation of the carpal bones and toes. The subject with RA may be resistant to DMARDs, in that the DMARDs are not effective or fully effective in treating symptoms. Further candidates for therapy according to this invention include those who have experienced an inadequate response to previous or current treatment with TNF inhibitors such as etanercept, infliximab and/or adalimumab because of toxicity or inadequate efficacy (for example, etanercept for 3 months at 25 mg twice a week or at least 4 infusions of infliximab at 3 mg/kg).

A patient with “active rheumatoid arthritis” means a patient with active and not latent symptoms of RA. Subjects with “early active rheumatoid arthritis” are those subjects with active RA diagnosed for at least 8 weeks but no longer than four years, according to the revised 1987 ACR criteria for the classification of RA.

Subjects with “early rheumatoid arthritis” are those subjects with RA diagnosed for at least eight weeks but no longer than four years, according to the revised 1987 ACR criteria for classification of RA. RA includes, for example, juvenile-onset RA, juvenile idiopathic arthritis (JIA), or juvenile RA (JRA).

Patients with “incipient RA” have early polyarthritis that does not fully meet ACR criteria for a diagnosis of RA, in association with the presence of RA-specific prognostic biomarkers such as anti-CCP and shared epitope. They include patients with positive anti-CCP antibodies who present with polyarthritis, but do not yet have a diagnosis of RA, and are at high risk for going on to develop bona fide ACR criteria RA (95% probability).

“Joint damage” is used in the broadest sense and refers to damage or partial or complete destruction to any part of one or more joints, including the connective tissue and cartilage, where damage includes structural and/or functional damage of any cause, and may or may not cause joint pain/arthalgia. It includes, without limitation, joint damage associated with or resulting from inflammatory joint disease as well as non-inflammatory joint disease. This damage may be caused by any condition, such as an autoimmune disease, especially arthritis, and most especially RA. Exemplary such conditions include acute and chronic arthritis, RA including juvenile-onset RA, JIA, or JRA, and stages such as rheumatoid synovitis, gout or gouty arthritis, acute immunological arthritis, chronic inflammatory arthritis, degenerative arthritis, type II collagen-induced arthritis, infectious arthritis, septic arthritis, Lyme arthritis, proliferative arthritis, psoriatic arthritis, Still's disease, vertebral arthritis, osteoarthritis, arthritis chronica progrediente, arthritis deformans, polyarthritis chronica primaria, reactive arthritis, menopausal arthritis, estrogen-depletion arthritis, and ankylosing spondylitis/rheumatoid spondylitis), rheumatic autoimmune disease other than RA, and significant systemic involvement secondary to RA (including but not limited to vasculitis, pulmonary fibrosis or Felty's syndrome). For purposes herein, joints are points of contact between elements of a skeleton (of a vertebrate such as an animal) with the parts that surround and support it and include, but are not limited to, for example, hips, joints between the vertebrae of the spine, joints between the spine and pelvis (sacroiliac joints), joints where the tendons and ligaments attach to bones, joints between the ribs and spine, shoulders, knees, feet, elbows, hands, fingers, ankles and toes, but especially joints in the hands and feet.

“Treatment” of a subject herein refers to both therapeutic treatment and prophylactic or preventative measures. Those in need of treatment include those already with RA or joint damage as well as those in which the RA or joint damage or the progress of RA or joint damage is to be prevented. Hence, the subject may have been diagnosed as having the RA or joint damage or may be predisposed or susceptible to the RA or joint damage, or may have RA or joint damage that is likely to progress in the absence of treatment. Treatment is successful herein if the RA or joint damage is alleviated or healed, or progression of RA or joint damage, including its signs and symptoms and structural damage, is halted or slowed down as compared to the condition of the subject prior to administration. Successful treatment further includes complete or partial prevention of RA or of the development of joint or structural damage. For purposes herein, slowing down or reducing RA or joint damage or the progression of joint damage is the same as arrest, decrease, or reversal of the RA or joint damage.

As used herein, the term “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

A “subject” herein is any single human subject, including a patient, eligible for treatment who is experiencing or has experienced one or more signs, symptoms, or other indicators of RA or joint damage, whether, for example, newly diagnosed or previously diagnosed and now experiencing a recurrence or relapse, or is at risk for RA or joint damage, no matter the cause. Intended to be included as a subject are any subjects involved in clinical research trials not showing any clinical sign of disease, or subjects involved in epidemiological studies, or subjects once used as controls. The subject may have been previously treated with a medicament for RA or joint damage, including a lymphotoxin receptor antagonist, or not so treated. The subject may be naïve to a second medicament being used when the treatment herein is started, i.e., the subject may not have been previously treated with, for example, an immunosuppressive agent such as MTX at “baseline” (i.e., at a set point in time before the administration of a first dose of antagonist in the treatment method herein, such as the day of screening the subject before treatment is commenced). Such “naïve” subjects are generally considered to be candidates for treatment with such second medicament.

“Clinical improvement” refers to prevention of further progress of RA or joint damage or any improvement in RA or joint damage as a result of treatment, as determined by various testing, including radiographic testing. Thus, clinical improvement may, for example, be determined by assessing the number of tender or swollen joints, the Psoriasis Assessment Severity Index, a global clinical assessment of the subject, assessing erythrocyte sedimentation rate, or assessing the amount of C-reactive protein level.

For purposes herein, a subject is in “remission” if he/she has no symptoms of RA or active joint damage, such as those detectable by the methods disclosed herein, and has had no progression of RA or joint damage as assessed at baseline or at a certain point of time during treatment. Those who are not in remission include, for example, those experiencing a worsening or progression of RA or joint damage. Such subjects experiencing a return of symptoms, including active RA or joint damage, are those who have “relapsed” or had a “recurrence.”

A “symptom” of RA or joint damage is any morbid phenomenon or departure from the normal in structure, function, or sensation, experienced by the subject and indicative of RA or joint damage, such as those noted above, including tender or swollen joints.

The expression “effective amount” refers to an amount of a medicament that is effective for treating RA or joint damage. This would include an amount that is effective in achieving a reduction in RA or joint damage as compared to baseline prior to administration of such amount as determined, e.g., by radiographic or other testing. An effective amount of a second medicament may serve not only to treat the RA or joint damage in conjunction with the antagonist herein, but also serve to treat undesirable effects, including side-effects or symptoms or other conditions accompanying RA or joint damage, including a concomitant or underlying disease or disorder.

“Total modified Sharp score” means a score obtained for assessment of radiographs using the method according to Sharp, as modified by Genant, Am. J. Med., 30:35-47 (1983). The primary assessment will be the change in the total Sharp-Genant score from screening. The Sharp-Genant score combines an erosion score and a joint space narrowing score of both hands and feet. Joint damage is measured in this test scoring by a mean change of less than the score at baseline (when patient is screened or tested before first administration of the antagonist herein).

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); NSAIDs; ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as CTX; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as MTX (oral or subcutaneous); anti-malarial agents such as chloroquine and hydroxychloroquine; sulfasalazine; leflunomide; cytokine antagonists such as cytokine antibodies or cytokine receptor antibodies including anti-interferon-α, -β, or -γ antibodies, anti-TNFα antibodies (infliximab (REMICADE®) or adalimumab), anti-TNF-alpha immunoadhesin (etanercept), anti-TNFβ antibodies, anti-IL-2 antibodies and anti-IL-2 receptor antibodies, and anti-IL-6 receptor antibodies and antagonists (such as ACTEMRA™ (tocilizumab)); anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 1990/08187); streptokinase; transforming growth factor-beta (TGFβ); streptodornase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251:430-432 (1991); WO 1990/11294; Ianeway, Nature, 341:482 (1989); and WO 1991/01133); BAFF antagonists such as anti-BAFF antibodies and anti-BR3 antibodies and zTNF4 antagonists (for review, see Mackay and Mackay, Trends Immunol., 23:113-115 (2002)); biologic agents that interfere with T cell helper signals, such as anti-CD40 receptor or anti-CD40 ligand (CD154), including blocking antibodies to CD40-CD40 ligand (e.g., Durie et al., Science, 261:1328-1330 (1993); Mohan et al., J. Immunol., 154:1470-1480 (1995)) and CTLA4-Fc (Finck et al., Science, 265:1225-1227 (1994)); and T-cell receptor antibodies (EP 340,109) such as T10B9. Some immunosuppressive agents herein are also DMARDs, such as MTX. Examples of preferred immunosuppressive agents herein include CTX, chlorambucil, azathioprine, leflunomide, MMF, or MTX.

The term “cytokine” is a generic term for proteins released by one cell population that act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-1b, IL-2, IL-3, IL-4, IL-5, IL-6, IL-7, IL-8, IL-9, IL-11, IL-12, IL-15, including PROLEUKIN® rIL-2; a tumor necrosis factor such as TNF-α or TNF-β (lymphotoxin); and other polypeptide factors including LIF and kit ligand (KL). As used herein, the term cytokine includes proteins from natural sources or from recombinant cell culture and biologically active equivalents of the native-sequence cytokines, including synthetically produced small-molecule entities and pharmaceutically acceptable derivatives and salts thereof. A “cytokine antagonist” is a molecule that inhibits or antagonizes such cytokines by any mechanism, including, for example, antibodies to the cytokine, antibodies to the cytokine receptor, and immunoadhesins.

Examples of “disease-modifying anti-rheumatic drugs” or “DMARDs” include hydroxycloroquine, sulfasalazine, MTX, leflunomide, etanercept, infliximab (plus oral and subcutaneous MTX), azathioprine, D-penicillamine, gold salts (oral), gold salts (intramuscular), minocycline, cyclosporine including cyclosporine A and topical cyclosporine, staphylococcal protein A (Goodyear and Silverman, J. Exp. Med., 197(9):1125-1139 (2003)), including salts and derivatives thereof, etc. A preferred DMARD herein is MTX.

Examples of “non-steroidal anti-inflammatory drugs” or “NSAIDs” include aspirin, acetylsalicylic acid, ibuprofen, naproxen, indomethacin, sulindac, tolmetin, COX-2 inhibitors such as celecoxib (CELEBREX®; 4-(5-(4-methylphenyl)-3-(trifluoromethyl)-1H-pyrazol-1-yl) benzenesulfonamide and valdecoxib (BEXTRA®), and meloxicam (MOBIC®), including salts and derivatives thereof, etc. Preferably, they are aspirin, naproxen, ibuprofen, indomethacin, or tolmetin.

“Corticosteroid” refers to any one of several synthetic or naturally occurring substances with the general chemical structure of steroids that mimic or augment the effects of the naturally occurring corticosteroids. Examples of synthetic corticosteroids include prednisone, prednisolone (including methylprednisolone, such as SOLU-MEDROL® methylprednisolone sodium succinate), dexamethasone or dexamethasone triamcinolone, hydrocortisone, and betamethasone. The preferred corticosteroids herein are prednisone, methylprednisolone, hydrocortisone, or dexamethasone.

A “medicament” is an active drug to treat RA or joint damage or the signs or symptoms or side effects of RA or joint damage.

The term “pharmaceutical formulation” refers to a sterile preparation that is in such form as to permit the biological activity of the medicament to be effective, and which contains no additional components that are unacceptably toxic to a subject to which the formulation would be administered.

A “sterile” formulation is aseptic or free from all living microorganisms and their spores.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

A “kit” is any manufacture (e.g a package or container) comprising at least one reagent, e.g., a medicament for treatment of RA or joint damage, or a probe for specifically detecting a biomarker gene or protein of the invention. The manufacture is preferably promoted, distributed, or sold as a unit for performing the methods of the present invention.

A “target audience” is a group of people or an institution to whom or to which a particular medicament is being promoted or intended to be promoted, as by marketing or advertising, especially for particular uses, treatments, or indications, such as individual patients, patient populations, readers of newspapers, medical literature, and magazines, television or internet viewers, radio or internet listeners, physicians, drug companies, etc.

The term “sample” or “test sample” as used herein generally refers to a biological sample. For example, a biological sample obtained from an individual. Examples of a biological sample are body fluid, body tissue, cells, tissue, cell culture, or other biological material. Body fluids are, e.g., lymph, sera, whole fresh blood, peripheral blood mononuclear cells, frozen whole blood, plasma (including fresh or frozen), urine, saliva, semen, synovial fluid and spinal fluid. Samples also include e.g., synovial tissue, skin, hair follicle, and bone marrow. Methods for obtaining tissue biopsies and body fluids from mammals are well known in the art. “Sample” and “biological sample” are used herein interchangeably.

For example, “serum sample” as used herein is e.g., a serum sample obtained from an individual. Methods for obtaining sera from mammals are well known in the art.

The term “synovial sample” as used herein is e.g., a synovial sample (e.g., fluid and/or tissue) obtained from an individual. Methods for obtaining synovial sample from mammals are well known in the art.

The term “biomarker” as used herein refers to an indicator of e.g., a normal and/or pathological state of a patient, which can be in response to therapeutic intervention. Examples of biomarkers include, but are not limited to a DNA, RNA, protein, carbohydrate, or glycolipid-based molecular marker, the expression or presence of in a biological sample can be detected by standard methods (or methods disclosed herein) and can be e.g., predictive and/or prognostic of the responsiveness of an RA patition to treatment with a LT antagonist. Such biomarkers contemplated by the present invention include, but are not limited to solLTαβ. In some embodiments, a biomarker (e.g., a specific mutation and/or SNP) is present in a test sample, and is not in a control or reference sample, or is present at a particular amount or level in the test sample that differs from the control or reference sample. In another embodiment, the expression of such a biomarker may be determined to be higher than that observed for a control sample. The terms “marker” and “biomarker” are used herein interchangeably. The terms “predictive” and “prognostic” as used herein, in the sense of meaning that the methods for prediction or prognostication are to allow the person practicing the method to select patients that are deemed likely to respond to treatment with a LT receptor antagonist and/or a LT antagonist.

The term “marker” or “biomarker” can also refer to an identifiable physical location on a chromosome, such as a restriction endonuclease recognition site or a gene, whose inheritance can be monitored. The marker may be an expressed region of a gene referred to as a “gene expression marker”, or some segment of DNA with no known coding function.

A “pharmacodynamic biomarker” or “PDB” as used herein refers to a biomarker that is detectable before, during, and/or after the administration of a therapeutic agent to a patient in need. Pharmacodynamic markers can e.g., provide the basis for a clinical trial or non-clinical trial assay which aid in determining the dosing and regimen, identifying patient subgroups or phenotypes that are responsive to the therapeutic agent, or selecting and developing a lead therapeutic agent. For example, lymphotoxin alpha-beta (LTαβ) may be used as a pharmacodynamic biomarker for identifying an RA patient subphenotype that is responsive to a LT antagonist, such as an anti-lymphotoxin alpha (LTα) antibody. Additionally, the PDBs can be used to monitor treatment with the drug.

The verbs “determine” and “assess” shall have the same meaning and are used interchangeably throughout the application.

An “effective response” of a patient or a patient's “responsiveness” to treatment with a lymphotoxin receptor antagonist and/or a LT antagonist and similar wording refers to the clinical or therapeutic benefit imparted to a patient at risk for or suffering from RA from or as a result of the treatment with the antagonist. Such benefit includes cellular or biological responses, a complete response, a partial response, a stable disease (without progression or relapse), or a response with a later relapse of the patient from or as a result of the treatment with the antagonist. For example, an effective response can be observed in a patient diagnosed with a lower amount of at least one of the biomarkers herein versus a patient not diagnosed with lower amounts of one or more of the biomarkers. The incidence of biomarker(s) herein effectively predicts, or predicts with high sensitivity, such effective response.

The expression “not responsive to,” as it relates to the reaction of subjects or patients to one or more of the medicaments that were previously administered to them, describes those subjects or patients who, upon administration of such medicament(s), did not exhibit any or adequate signs of treatment of the disorder for which they were being treated, or they exhibited a clinically unacceptably high degree of toxicity to the medicament(s), or they did not maintain the signs of treatment after first being administered such medicament(s), with the word treatment being used in this context as defined herein. The phrase “not responsive” includes a description of those subjects who are resistant and/or refractory to the previously administered medication(s), and includes the situations in which a subject or patient has progressed while receiving the medicament(s) that he or she is being given, and in which a subject or patient has progressed within 12 months (for example, within six months) after completing a regimen involving the medicament(s) to which he or she is no longer responsive. The non-responsiveness to one or more medicaments thus includes subjects who continue to have active disease following previous or current treatment therewith. For instance, a patient may have active disease activity after about one to three months of therapy with the medicament(s) to which they are non-responsive. Such responsiveness may be assessed by a clinician skilled in treating the disorder in question.

By “reducing the risk of a negative side effect” is meant reducing the risk of a side effect resulting from treatment with the antagonist herein to a lower extent than the risk observed resulting from treatment of the same patient or another patient with a previously administered medicament. Such side effects include those set forth above regarding toxicity, and are preferably infection, cancer, heart failure, or demyelination.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to various embodiments herein, one may use the results of an analytical assay to determine whether a specific therapeutic regimen using a a lymphotoxin receptor antagonist and/or a LT antagonist, such as an anti-LTα antibody, should be performed.

The “amount” or “level” of a biomarker associated with an increased clinical benefit to a RA patient or patient with joint damage is a detectable level in a biological sample. These can be measured by methods known to the expert skilled in the art and also disclosed by this invention. The expression level or amount of biomarker assessed can be used to determine the response to the treatment.

The terms “level of expression” or “expression level” in general are used interchangeably and generally refer to the amount of a polynucleotide or an amino acid product or protein in a biological sample. “Expression” generally refers to the process by which gene-encoded information is converted into the structures present and operating in the cell. Therefore, according to the invention “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

An “algorithm” as used in the methods and systems herein is a specific set of instructions or a definite list of well-defined instructions for carrying out a procedure, typically proceeding through a well-defined series of successive states, and eventually terminating in an end-state, in this case, a binary answer of yes or no to the amount(s) of the cytokine(s).

As used herein, the term “covariate” refers to certain variables or information relating to a patient. The clinical endpoints are frequently considered in regression models, where the endpoints represent the dependent variable and the biomarkers represent the main or target independent variables (regressors). If additional variables from the clinical data pool are considered, they are denoted as (clinical) covariates.

The term “clinical covariate” is used herein to describe all clinical information about the patient, which is in general available at baseline. These clinical covariates comprise demographic information like sex, age, etc., other anamnestic information, concomitant diseases, concomitant therapies, results of physical examinations, common laboratory parameters obtained, known properties of the RA or joint damage, information quantifying the extent of RA disease, clinical performance scores like ECOG or Karnofsky index, clinical disease staging, timing and result of pretreatments, disease history, as well as all similar information that may be associated with the clinical response to treatment.

As used herein, the term “raw analysis” or “unadjusted analysis” refers to regression analyses, wherein besides the considered biomarkers, no additional clinical covariates are used in the regression model, neither as independent factors nor as stratifying covariate.

As used herein, the term “adjusted by covariates” refers to regression analyses, wherein besides the considered biomarkers, additional clinical covariates are used in the regression model, either as independent factors or as stratifying covariate.

As used herein, the term “univariate” refers to regression models or graphical approaches wherein, as an independent variable, only one of the target biomarkers is part of the model. These univariate models can be considered with and without additional clinical covariates.

As used herein, the term “multivariate” refers to regression models or graphical approaches wherein, as independent variables, more than one of the target biomarkers is part of the model. These multivariate models can be considered with and without additional clinical covariates.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide, followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The terms “splicing” and “RNA splicing” are used interchangeably and refer to RNA processing that removes introns and joins exons to produce mature mRNA with continuous coding sequence that moves into the cytoplasm of an eukaryotic cell.

In theory, the term “exon” refers to any segment of an interrupted gene that is represented in the mature RNA product (B. Lewin. Genes IV Cell Press, Cambridge Mass. 1990). In theory the term “intron” refers to any segment of DNA that is transcribed but removed from within the transcript by splicing together the exons on either side of it. Operationally, exon sequences occur in the mRNA sequence of a gene as defined by Ref. SEQ ID numbers. Operationally, intron sequences are the intervening sequences within the genomic DNA of a gene, bracketed by exon sequences and having GT and AG splice consensus sequences at their 5′ and 3′ boundaries.

The word “label” when used herein refers to a compound or composition that is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable. The term is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “naked antibody” is an antibody that is not conjugated to a heterologous molecule, such as a small molecule or radiolabel.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step. The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant dmain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

An “amino acid sequence variant” antibody herein is an antibody with an amino acid sequence which differs from a main species antibody. Ordinarily, amino acid sequence variants will possess at least about 70% homology with the main species antibody, and preferably, they will be at least about 80%, more preferably at least about 90% homologous with the main species antibody. The amino acid sequence variants possess substitutions, deletions, and/or additions at certain positions within or adjacent to the amino acid sequence of the main species antibody. Examples of amino acid sequence variants herein include an acidic variant (e.g. deamidated antibody variant), a basic variant, an antibody with an amino-terminal leader extension (e.g. VHS-) on one or two light chains thereof, an antibody with a C-terminal lysine residue on one or two heavy chains thereof, etc., and includes combinations of variations to the amino acid sequences of heavy and/or light chains. The antibody variant of particular interest herein is the antibody comprising an amino-terminal leader extension on one or two light chains thereof, optionally further comprising other amino acid sequence and/or glycosylation differences relative to the main species antibody.

A “glycosylation variant” antibody herein is an antibody with one or more carbohydrate moieities attached thereto which differ from one or more carbohydrate moieties attached to a main species antibody. Examples of glycosylation variants herein include antibody with a G1 or G2 oligosaccharide structure, instead a G0 oligosaccharide structure, attached to an Fc region thereof, antibody with one or two carbohydrate moieties attached to one or two light chains thereof, antibody with no carbohydrate attached to one or two heavy chains of the antibody, etc., and combinations of glycosylation alterations.

Where the antibody has an Fc region, an oligosaccharide structure may be attached to one or two heavy chains of the antibody, e.g. at residue 299 (298, Eu numbering of residues). For pertuzumab, G0 was the predominant oligosaccharide structure, with other oligosaccharide structures such as G0-F, G-1, Man5, Man6, G1-1, G1(1-6), G1(1-3) and G2 being found in lesser amounts in the pertuzumab composition.

Unless indicated otherwise, a “G1 oligosaccharide structure” herein includes G-1, G1-1, G1(1-6) and G1(1-3) structures.

An “amino-terminal leader extension” herein refers to one or more amino acid residues of the amino-terminal leader sequence that are present at the amino-terminus of any one or more heavy or light chains of an antibody. An exemplary amino-terminal leader extension comprises or consists of three amino acid residues, VHS, present on one or both light chains of an antibody variant.

A “deamidated” antibody is one in which one or more asparagine residues thereof has been derivatized, e.g. to an aspartic acid, a succinimide, or an iso-aspartic acid.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

By “solid phase” or “solid support” is meant a non-aqueous matrix to which a polypeptide, nucleic acid, antibody or Ihh, DefA5 and/or DefA6 binding agent-of the present invention can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes.

A “small molecule” or “small organic molecule” is defined herein to have a molecular weight below about 500 Daltons.

The expression “effective amount” refers to an amount of a medicament that is effective for treating RA or joint damage. This would include an amount that is effective in achieving a reduction in RA or joint damage as compared to baseline prior to administration of such amount as determined, e.g., by radiographic or other testing. An effective amount of a second medicament may serve not only to treat the RA or joint damage in conjunction with the antagonist herein, but also serve to treat undesirable effects, including side-effects or symptoms or other conditions accompanying RA or joint damage, including a concomitant or underlying disease or disorder. An “effective amount” may be determined empirically and in a routine manner, in relation to this purpose.

The terms “level of expression” or “expression level” are used interchangeably and generally refer to the amount of a polynucleotide or an amino acid product or protein in a biological sample. “Expression” generally refers to the process by which gene-encoded information is converted into the structures present and operating in the cell. Therefore, according to the invention “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

The term “overexpression” as used herein, refers to cellular gene expression levels of a tissue that is higher than the normal expression levels for that tissue. The term “underexpression” as used herein, refers to cellular gene expression levels of a tissue that is lower than the normal expression levels for that tissue. In either case, the higher or lower expression is significantly different from normal expression under controlled conditions of the study.

A “control” includes a sample obtained from an individual for use in determining base-line or normal expression of a gene or activity of a protein in a patientmammal. Accordingly, a control sample may be obtained by a number of means including from individuals not affected by a rheumatoid arthritis (as determined by standard techniques); e.g., a control sample of a subject not experiencing RA; a control sample from a subject not having RA; or a control sample from a subject not suspected of being at risk for RA. A control also includes a previously established standard. Accordingly, any test or assay conducted according to the invention may be compared with the established standard and it may not be necessary to obtain a control sample for comparison each time.

### III. General Description of the Invention

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

The present invention relates to a soluble lymphotoxin (solLT) and methods of using the solLT as a biomarker in the treatment of autoimmune disease. More particularly, the present invention relates to soluble lymphotoxin alpha-beta (solLTαβ) and methods of using this solLTαβ as a biomarker in the treatment of rheumatoid arthritis (RA).

The compositions and methods of the present invention provide for convenient, efficient, and potentially cost-effective means to obtain information that aids in patient treatment decisions for autoimmune diseases such as RA. For example, the present invention provides methods of using the amount of solLTαβ in a patient with RA to assess or identify appropriate or effective therapies for treating the patient.

A. Soluble LTalpha-beta (solLTαβ) Compositions and Methods

The present invention provides soluble LTalpha-beta (solLTαβ) compositions and methods for use in obtaining information regarding the treatment of autoimmune diseases, e.g. rheumatoid arthritis (RA). For example, the present invention provides methods of assessing the responsiveness of a patient, having an autoimmune disease, to treatment with an LT antagonist, the method comprising assessing or determining the level of a soluble LTαβ (solLTαβ) in the patient, where an increased amount of the solLTαβ in the treated patient, as compared to the amount in the untreated patient, is indicative of responsiveness to treatment with the LT antagonist.

The amount or level of solLTαβ may be determined using a variety of standard assay formats, including assays for detecting protein or nucleic acids. In some emodiments, the assay format detects the amount of solLTαβ protein or RNA, and an activity thereof.

In one embodiment, the present invention provides a method of predicting whether a patient with RA will respond to treatment with a LT antagonist, comprising assessing the amount of solLTαβ in the patient, where the amount of solLTαβ is predictive of whether the patient will respond to treatment with the LT antagonist. In one embodiment, serum and/or synovial fluid is obtained from the patient and subjected to an assay to assess the amount of biomarkers present in the patient. In some embodiments, the threshold or baseline amount may be determined based upon a control sample. In one aspect the control sample is a synovial fluid sample from an osteoarthritis patient's affected joint or from the RA patient's affected joint prior to treatment. In another aspect the control sample is from a normal donor serum sample or a pre-treatment sample from the RA patient.

In another embodiment, the invention provides a method of specifying a LT antagonist for use in a RA patient subpopulation, the method comprising providing instruction to administer the LT antagonist to a patient subpopulation having an amount of solLTαβ that correlates with or is indicative of RA.

In a further aspect, the invention provides a system for analyzing responsiveness of a patient with RA to treatment with a LT antagonist comprising: reagents to detect in a sample from the patient an amount of solLTαβ; hardware to perform detection of the biomarkers; and computational means to perform an algorithm to determine if the patient is susceptible or responsive to the treatment.

The reagents to detect the solLTαβ may be, for example, antibodies, polynucleotides, and other molecules that bind to solLTαβ. The hardware is preferably a machine or computer to perform the detection step, and the computational means may be by, for example, computer or machine.

The invention further provides a method for selecting a therapy for a patient or a patient population with RA comprising assessing the amount of solLTαβ in the patient or patient population, wherein the amount of solLTαβ indicates the patient will be responsive to the therapy. In one embodiment, the amount of solLTαβ in the patient serum or synovial fluid or tissue is assessed. In one embodiment, the method further comprises administering the LT antagonist to the patient. In a further embodiment, the antagonist is an anti-LTα antibody.

In another embodiment, the invention provides a method for selecting a patient with RA for treatment with a LT antagonist comprising assessing the amount of solLTαβ in the patient, wherein the amount of solLTαβ indicates the patient will be responsive to treatment with the LT antagonist. In one embodiment, the amount of solLTαβ in the patient serum or synovial fluid or tissue is assessed. In one embodiment, the method further comprises administering the LT antagonist to the patient. In a further embodiment, the antagonist is an anti-LTα antibody.

In another embodiment, the invention provides a method for identifying a patient with RA for treatment with a LT antagonist comprising assessing the amount of solLTαβ in the patient, wherein the amount of solLTαβ indicates the patient will be responsive to treatment with the LT antagonist. In one embodiment, the amount of solLTαβ in the patient serum or synovial fluid or tissue is assessed. In one embodiment, the method further comprises administering the LT antagonist to the patient. In a further embodiment, the antagonist is an anti-LTα antibody.

In another embodiment, the invention provides a method for monitoring the responsiveness of an RA patient to treatment with a LT antagonist, comprising assessing the amount of solLTαβ in the patient, wherein the amount of solLTαβ is indicative of the responsiveness of the patient to treatment with the LT antagonist. In one embodiment, the amount of solLTαβ in the patient serum or synovial fluid or tissue is assessed. In a one embodiment, the antagonist is an anti-LTα antibody.

One of skill in the medical arts, particularly pertaining to the application of diagnostic tests and treatment with therapeutics, will recognize that biological systems are somewhat variable and not always entirely predictable, and thus many good diagnostic tests or therapeutics are occasionally ineffective. Thus, it is ultimately up to the judgment of the attending physician to determine the most appropriate course of treatment for an individual patient, based upon test results, patient condition and history, and his or her own experience. There may even be occasions, for example, when a physician will choose to treat a patient with a LT antagonist even when a patient is not predicted to be particularly sensitive to LT antagonists, based on data from diagnostic tests or from other criteria, particularly if all or most of the other obvious treatment options have failed, or if some synergy is anticipated when given with another treatment.

The present invention further provides a method of identifying a biomarker whose expression level is predictive of the effective responsiveness of a particular patient with RA to a LT antagonist comprising: (a) measuring the expression level of a candidate biomarker in a panel of cells that displays a range of sensitivities to a LT antagonist, and (b) identifying a correlation between the expression level of or presence of said candidate biomarker in the cells and the sensitivity of a patient with RA to effective responsiveness to the LT antagonist, wherein the correlation indicates that the expression level or presence of said biomarker is predictive of the responsiveness of the patient to treatment by a LT antagonist. In one embodiment of this method the panel of cells is a panel of RA samples prepared from samples derived from patients or experimental animal models. In an additional embodiment the panel of cells is a panel of cell lines in mouse xenografts, wherein responsiveness can, for example, be determined by monitoring a molecular marker of responsiveness.

The present invention also provides a method of identifying a biomarker that is diagnostic for more effective treatment of RA with a LT antagonist comprising: (a) measuring the level of a candidate biomarker in samples from patients with RA, and (b) identifying a correlation between the expression level of or presence of said candidate biomarker in the sample from the patient with the effectiveness of treatment of the RA with a LT antagonist, wherein the correlation indicates that said biomarker is diagnostic for more effective treatment of the RA with a LT antagonist.

In another aspect, the present invention provides a method of identifying a biomarker that is diagnostic for prolonged symptom-free status of a patient with RA when treated with a LT antagonist comprising: (a) measuring the level of the candidate biomarker in samples from patients with RA, and (b) identifying a correlation between the expression level, seropositivity, or presence of said candidate biomarker in the sample from the patient with prolonged symptom-free status of that patient when treated with a LT antagonist, wherein the correlation of a biomarker with prolonged symptom-free status in said patients indicates said biomarker is diagnostic for prolonged symptom-free status of a patient with RA when treated with a LT antagonist.

The effectiveness of treatment in the preceding methods can, for example, be determined by using the ACR and/or European League Against Rheumatism (EULAR) clinical response parameters in the patients with RA, or by assaying a molecular determinant of the degree of RA in the patient.

In all the methods described herein the sample is taken from a patient who is suspected to have, or is diagnosed to have RA, and hence is likely in need of treatment. For assessment of marker expression, patient samples, such as those containing cells, or proteins or nucleic acids produced by these cells, may be used in the methods of the present invention. In the methods of this invention, the level of a biomarker can be determined by assessing the amount (e.g. absolute amount or concentration) of the markers in a sample, preferably assessed in bodily fluids or excretions containing detectable levels of biomarkers. In some embodiments, synovial fluid, synovial tissue, and/or serum is used for assessment of the amount of solLTαβ. “Blood” as used herein includes, whole blood, plasma, serum, or any derivative of blood. Other bodily fluids or secretions are useful as samples in the present invention including, e.g., urine, saliva, stool, pleural fluid, lymphatic fluid, sputum, ascites, prostatic fluid, cerebrospinal fluid (CSF), or any other bodily secretion or derivative thereof. Assessment of a biomarker in such bodily fluids or excretions can sometimes be preferred in circumstances where an invasive sampling method is inappropriate or inconvenient. However, the sample to be tested herein is preferably synovial tissue, synovial fluid, blood/serum, or any combination thereof.

The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, biopsies may also be subjected to post-collection preparative and storage techniques, e.g., fixation.

In one embodiment, where one or more of the biomarkers described herein are found to be present in a sample from the patient at level(s) no greater than predetermined threshold level(s) for each biomarker, the patient from whom the sample was procured is concluded to be a candidate for therapy with a LT antagonist as disclosed herein. The level of biomarker protein can be determined using methods well known to those skilled in the art.

As to physical and quantitative tests for detection of protein biomarkers, such as LTαβ and/or LTα for example, various protein assays are available. For example, the sample may be contacted with an antibody specific for said biomarker under conditions sufficient for an antibody-biomarker complex to form, and then detecting said complex. The presence of the protein biomarker may be accomplished in a number of ways, such as by Western blotting (with or without immunoprecipitation), 2-dimensional SDS-PAGE, immunoprecipitation, fluorescence activated cell sorting (FACS), flow cytometry, and ELISA procedures for assaying a wide variety of tissues and samples, including plasma or serum. A wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g., from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e., radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase, and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

An enzyme immunoassay (EIA) and serological assay, including a second-generation ELISA (IMMUNOSCAN RA™), as well as an agglutination assay (Latex and Waaler-Rose) and specific ELISA (IgM, IgG and IgA) may also be used. Commercially available ELISAs can be used, including IMMUNOSCAN RA™ (Eurodiagnostica, The Netherlands), Inova Diagnostics and Axis-Shield Diagnostics. Detection can be using 3 synthetic citrullinated peptide variants.

Abreu et al., “Multiplexed immunoassay for detection of rheumatoid factors by FIDIS Technology” Annals of the New York Academy of Sciences 1050 (Autoimmunity), 357-363 (2005) compares FIDIS RHEUMA™, a multiplexed immunoassay designed for simultaneous detection of IgM class RF directed against Fc determinants of IgG from humans and animals, with agglutination and ELISA and evaluates the clinical sensitivity and specificity of biological markers for RA. FIDIS technology was employed using the LUMINEX™ system and consisted of distinct color-coded microsphere sets, a flow cytometer, and digital signal processing hardware and software. Agglutination and ELISA tests can be performed with commercial kits. For human specificity, FIDIS was compared with latex agglutination and ELISA. For animal specificity, FIDIS was compared with Waaler-Rose and ELISA. Detection of IgG anti-CCP by ELISA by immunofluorescence was also determined. Dubois-Galopin et al., “Evaluation of a new fluorometric immunoassay for the detection of anti-cyclic citrullinated peptide autoantibodies in rheumatoid arthritis” Annales de Biologie Clinique, 64(2):162-165 (2006) evaluated the measurement of anti-CCP antibodies by a new fluorescent-enzyme immunoassay, called EliA CCP™, fully automated onto UniCAP 100ε. This compares well with an ELISA method (Euroimmun).

Methods for detecting genetic biomarkers desired to be assessed in addition to the protein biomarker(s) (for example, polymorphisms) include protocols that examine the presence and/or expression of a SNP, for example, in a sample. Tissue or cell samples from mammals can be conveniently assayed for, e.g., genetic-marker mRNAs or DNAs using Northern, dot-blot, or polymerase chain reaction (PCR) analysis, array hybridization, RNase protection assay, or using DNA SNP chip microarrays, which are commercially available, including DNA microarray snapshots. For example, real-time PCR (RT-PCR) assays such as quantitative PCR assays are well known in the art. In an illustrative embodiment of the invention, a method for detecting a SNP mRNA in a biological sample comprises producing cDNA from the sample by reverse transcription using at least one primer; amplifying the cDNA so produced using a SNP polynucleotide as sense and antisense primers to amplify SNP cDNAs therein; and detecting the presence of the amplified SNP cDNA. In addition, such methods can include one or more steps that allow one to determine the levels of SNP mRNA in a biological sample (e.g., by simultaneously examining the levels a comparative control mRNA sequence of a “housekeeping” gene such as an actin family member). Optionally, the sequence of the amplified SNP cDNA can be determined.

In one specific embodiment, genotyping of a polymorphism can be performed by RT-PCR technology, using the TAQMAN™ 5′-allele discrimination assay, a restriction fragment-length polymorphism PCR-based analysis, or a PYROSEQUENCER™ instrument. In addition, the method of detecting a genetic variation or polymorphism set forth in U.S. Pat. No. 7,175,985 may be used. In this method a nucleic acid is synthesized utilizing the hybridized 3′-end, which is synthesized by complementary strand synthesis, on a specific region of a target nucleotide sequence existing as the nucleotide sequence of the same strand as the origin for the next round of complementary strand synthesis.

Probes used for PCR may be labeled with a detectable marker, such as, for example, a radioisotope, fluorescent compound, bioluminescent compound, a chemiluminescent compound, metal chelator, or enzyme. Such probes and primers can be used to detect the presence of a SNP in a sample and as a means for detecting a cell expressing SNP-encoded proteins. As will be understood by the skilled artisan, a great many different primers and probes may be prepared based on known sequences and used effectively to amplify, clone, and/or determine the presence and/or levels of SNP mRNAs.

Other methods include protocols that examine or detect mRNAs in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes that have potential to be expressed in certain disease states may be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment (see, e.g., WO 2001/75166). See, for example, U.S. Pat. No. 5,700,637, U.S. Pat. No. 5,445,934, and U.S. Pat. No. 5,807,522, Lockart, Nature Biotechnology, 14:1675-1680 (1996); and Cheung et al., Nature Genetics, 21(Suppl):15-19 (1999) for a discussion of array fabrication.

In addition, the DNA profiling and SNP detection method utilizing microarrays described in EP 1,753,878 may be employed. This method rapidly identifies and distinguishes between different DNA sequences utilizing short tandem repeat (STR) analysis and DNA microarrays. In an embodiment, a labeled STR target sequence is hybridized to a DNA microarray carrying complementary probes. These probes vary in length to cover the range of possible STRs. The labeled single-stranded regions of the DNA hybrids are selectively removed from the microarray surface utilizing a post-hybridization enzymatic digestion. The number of repeats in the unknown target is deduced based on the pattern of target DNA that remains hybridized to the microarray.

One example of a microarray processor is the Affymetrix GENECHIP® system, which is commercially available and comprises arrays fabricated by direct synthesis of oligonucleotides on a glass surface. Other systems may be used as known to one skilled in the art.

Other methods for determining the level of the biomarker besides RT-PCR or another PCR-based method include proteomics techniques, as well as individualized genetic profiles that are necessary to treat RA based on patient response at a molecular level. The specialized microarrays herein, e.g., oligonucleotide microarrays or cDNA microarrays, may comprise one or more biomarkers having expression profiles that correlate with either sensitivity or resistance to one or more anti-CD20 antibodies. Additionally, SNPs can be detected using electronic circuitry on silicon microchips, as disclosed, for example, in WO 2000/058522.

Identification of biomarkers that provide rapid and accessible readouts of efficacy, drug exposure, or clinical response is increasingly important in the clinical development of drug candidates. Embodiments of the invention include measuring changes in the levels of secreted proteins, or plasma biomarkers, which represent one category of biomarker. In one aspect, plasma samples, which represent a readily accessible source of material, serve as surrogate tissue for biomarker analysis.

Many references are available to provide guidance in applying the above techniques (Kohler et al., Hybridoma Techniques (Cold Spring Harbor Laboratory, New York, 1980); Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982); and Zola, Monoclonal Antibodies: A Manual of Techniques, pp. 147-158 (CRC Press, Inc., 1987)). Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, N.Y. 11803-2500. Typical protocols for evaluating the status of genes and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis).

For use in detection of the biomarkers, kits or articles of manufacture are also provided by the invention. Such kits can be used to determine if a subject with RA will be effectively responsive to a LT antagonist. These kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be an antibody or polynucleotide specific for a protein or autoantibody marker or a gene or message, respectively. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, e.g., avidin or streptavidin, bound to a reporter molecule, such as an enzymatic, florescent, or radioisotope label.

Such kit will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above.

The kits of the invention have a number of embodiments. A typical embodiment is a kit comprising a container, a label on said container, and a composition contained within said container, wherein the composition includes a primary antibody that binds to a protein or autoantibody biomarker, and the label on said container indicates that the composition can be used to evaluate the presence of such proteins or antibodies in a sample, and wherein the kit includes instructions for using the antibody for evaluating the presence of biomarker proteins in a particular sample type. The kit can further comprise a set of instructions and materials for preparing a sample and applying antibody to the sample. The kit may include both a primary and secondary antibody, wherein the secondary antibody is conjugated to a label, e.g., an enzymatic label.

Another embodiment is a kit for detecting the biomarker(s) along with a genetic polymorphism biomarker that comprises a first container, a label on said container, and a composition contained within said container, wherein the composition includes a reagent to detect the biomarker(s) as noted above, a second container, a label on said container, and a composition contained within said second container, wherein the composition includes one or more polynucleotides that hybridize to a complement of the polynucleotide polymorphism being detected under stringent conditions, and the label on said first container indicates that the composition can be used to evaluate the presence of one or more of the biomarkers described herein in a sample, and the label on said second container indicates that the composition can be used to evaluate the presence of a SNP in a sample (the sample being the same or different from the one containing the cytokine(s)), and wherein the kit includes instructions for using the reagent for detecting the amount(s) of biomarker(s) in a particular sample and instructions for using the polynucleotide(s) for evaluating the presence of the SNP RNA or DNA in a particular sample type.

Other optional components of the kit include one or more buffers (e.g., block buffer, wash buffer, substrate buffer, etc.), other reagents such as substrate (e.g., chromogen) that is chemically altered by an enzymatic label, epitope retrieval solution, control samples (positive and/or negative controls), control slide(s), etc. Kits can also include instructions for interpreting the results obtained using the kit.

In further specific embodiments, for antibody-based kits, the kit can comprise, for example: (1) a first antibody (e.g., attached to a solid support) that binds to a biomarker protein; and, optionally, (2) a second, different antibody that binds to either the protein or the first antibody and is conjugated to a detectable label.

For kits that also detect genes (oligonucleotide-based kits), the kit can also comprise, for example: (1) an oligonucleotide, e.g., a detectably labeled oligonucleotide, which hybridizes to a nucleic acid sequence encoding a biomarker protein or (2) a pair of primers useful for amplifying a biomarker nucleic acid molecule. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples that can be assayed and compared to the test sample. Each component of the kit can be enclosed within an individual container and all of the various containers can be within a single package, along with instructions for interpreting the results of the assays performed using the kit.

B. Statistics

As used herein, the general form of a prediction rule consists in the specification of a function of one or multiple biomarkers potentially including clinical covariates to predict response or non-response, or more generally, predict benefit or lack of benefit in terms of suitably defined clinical endpoints.

The simplest form of a prediction rule consists of a univariate model without covariates, wherein the prediction is determined by means of a cutoff or threshold. This can be phrased in terms of the Heaviside function for a specific cutoff c and a biomarker measurement x, where the binary prediction A or B is to be made, then

If H (x−c)=0, then predict A.

If H (x−c)=1, then predict B.

This is the simplest way of using univariate biomarker measurements in prediction rules. If such a simple rule is sufficient, it allows for a simple identification of the direction of the effect, i.e., whether high or low expression levels are beneficial for the patient.

The situation can be more complicated if clinical covariates need to be considered and/or if multiple biomarkers are used in multivariate prediction rules. The two hypothetical examples below illustrate the issues involved:

Covariate Adjustment (Hypothetical Example): For a biomarker X it is found in a clinical trial population that high expression levels are associated with a worse clinical response (univariate analysis). A closer analysis shows that there are two types of RA clinical response in the population, one of which possesses a worse response than the other one and at the same time the biomarker expression for this overall RA group is generally higher. An adjusted covariate analysis reveals that for each of the RA types the relation of clinical benefit and clinical response is reversed, i.e., within the RA types, lower expression levels are associated with better clinical response. The overall opposite effect was masked by the covariate RA type—and the covariate adjusted analysis as part of the prediction rule reversed the direction.

Multivariate Prediction (Hypothetical Example): For a biomarker X it is found in a clinical trial population that high expression levels are slightly associated with a worse clinical response (univariate analysis). For a second biomarker Y a similar observation was made by univariate analysis. The combination of X and Y revealed that a good clinical response is seen if both biomarkers are low. This makes the rule to predict benefit if both biomarkers are below some cutoffs (AND—connection of a Heaviside prediction function). For the combination rule, a simple rule no longer applies in a univariate sense; for example, having low expression levels in X will not automatically predict a better clinical response.

These simple examples show that prediction rules with and without covariates cannot be judged on the univariate level of each biomarker. The combination of multiple biomarkers plus a potential adjustment by covariates does not allow assigning simple relationships to single biomarkers. Since the marker genes, in particular in serum, may be used in multiple-marker prediction models potentially including other clinical covariates, the direction of a beneficial effect of a single marker gene within such models cannot be determined in a simple way, and may contradict the direction found in univariate analyses, i.e., the situation as described for the single marker gene.

C. Methods of Diagnosis Using Soluble LTalpha-Beta (solLTαβ) as a Biomarker

The methods of the present invention are valuable tools for providing information concerning methods of treating autoimmune diseases, e.g., rheumatoid arthritis (RA).

In one embodiment, the methods provided herein include the step of determining the amount of solLTαβ in a sample from an RA patient. The methods of the present invention may further include the step of manipulating or testing a sample from an RA patient. In one embodiment, the manipulating step includes contacting a sample with a reagent to detect the amount of solLTαβ. In another embodiment, the reagent is a nucleic acid, a polypeptide, an antibody or a solLTαβ-reactive fragment thereof, a recombinant.

For example, a method of detecting the differential expression of an IBD marker in a biological sample comprises first contacting the sample with an anti-IBD marker antibody, an IBD marker-reactive fragment thereof, or a recombinant protein containing an antigen-binding region of an anti-IBD marker antibody; and then detecting the binding of an IBD marker protein in the sample.

Measurement of biomarker expression or protein levels may be performed by using a software program executed by a suitable processor. Suitable software and processors are well known in the art and are commercially available. The program may be embodied in software stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

Following the measurement or obtainment of the level of solLTαβ, the assay results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. In some embodiments, the assays will be performed or the assay results analyzed in a country or jurisdiction which differs from the country or jurisdiction to which the results or diagnoses are communicated.

In a preferred embodiment, a diagnosis, prediction and/or treatment recommendation based on the solLTαβ level in a patient is communicated to the patient as soon as possible after the assay is completed and the diagnosis and/or prediction is generated. The results and/or related information may be communicated to the patient by the patient's treating physician. Alternatively, the results may be communicated directly to a patient by any means of communication, including writing, such as by providing a written report, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761, the entire contents of which are incorporated by reference herein; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

To facilitate diagnosis, the level of solLTαβ can be displayed on a display device, contained electronically, or in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history.

The methods of this invention, when practiced for commercial diagnostic purposes generally produce a report or summary of the normalized levels of one or more of the biomarkers described herein. The methods of this invention will produce a report comprising one or more predictions concerning a patient and a LT antagonist treatment including, but not limited to, suitability for treatment, responsiveness to treatment, therapeutic efficacy of treatment, safety of treatment, or any combination thereof. In another embodiment, the reports may concern a prediction regarding a patient who has not been administered a LT antagonist treatment or a prediction regarding a patient who has been administered a LT antagonist treatment.

The methods and reports of this invention can further include storing the report in a database. Alternatively, the method can further create a record in a database for the subject and populate the record with data. In one embodiment the report is a paper report, in another embodiment the report is an auditory report, in another embodiment the report is an electronic record. It is contemplated that the report is provided to a physician and/or the patient. The receiving of the report can further include establishing a network connection to a server computer that includes the data and report and requesting the data and report from the server computer. The methods provided by the present invention may also be automated in whole or in part.

D. RA Patients—Use of Soluble LTalpha-Beta (solLTαβ) as a Biomarker

The present invention provides methods of providing information about RA patients who have been treated or are undergoing treatment with a therapeutically effective amount of a LT antagonist by detecting or determining in a patient sample the amount of solLTαβ, wherein the amount of solLTαβ indicates that the patient is responsive or likely to be responsive to treatment with the LT antagonist. An example of such an amount of solLTαβ is 20-800 pg/ml in patient serum or 20-400 pg/ml in patient synovial fluid.

In another embodiment, the invention provides a method wherein the detected amount of solLTαβ in a patient sample is: diagnostic, predictive or prognositic of RA or progression of RA or risk of RA. An example of such an amount of solLTαβ is at least 50 pg/ml, at least 100 pg/ml, at least 200 pg/ml, at least 300 pg/ml, at least 400 pg/ml, or at least 500 pg/ml.

The effectiveness of LT antagonist treatment in the preceding methods can, for example, be determined by using the ACR and/or EULAR clinical response parameters in the patients with RA, or by assaying a molecular determinant of the degree of RA in the patient. Thus, for example, a clinician may use any of several methods known in the art to measure the effectiveness of a particular dosage scheme of a LT antagonist. For example, x-ray technology can be used to determine the extent of joint destruction and damage in the patient, and the scale of ACR20, ACR50, and ACR70 can be used to determine relative effective responsiveness to the therapy. Dosage regimens may be adjusted to provide the optimum desired response (e.g., a therapeutic response). For example, a dose may be administered, several divided doses may be administered over time, or the dose may be proportionally reduced or increased as indicated by exigencies of the therapeutic situation.

Once the patient population most responsive to treatment with the antagonist has been identified, treatment with the antagonist herein, alone or in combination with other medicaments, results in an improvement in the RA or joint damage, including signs or symptoms thereof. For instance, such treatment may result in an improvement in ACR measurements relative to a patient treated with the second medicament only (e.g., an immunosuppressive agent such as MTX), and/or may result in an objective response (partial or complete, preferably complete) as measured by ACR. Moreover, treatment with the combination of an antagonist herein and at least one second medicament preferably results in an additive, more preferably synergistic (or greater than additive) therapeutic benefit to the patient. Preferably, in this method the timing between at least one administration of the second medicament and at least one administration of the antagonist herein is about one month or less, more preferably, about two weeks or less.

It will be appreciated by one of skill in the medical arts that the exact manner of adjusting or modifying the administration to the patient a therapeutically effective amount of a LT antagonist following a diagnosis of a patient's likely responsiveness to the antagonist will be at the discretion of the attending physician. The mode of administration, including dosage, combination with other anti-RA agents, timing and frequency of administration, and the like, may be affected by the extent of the diagnosis of the patient's likely responsiveness to such antagonist, as well as the patient's condition and history.

The composition comprising an antagonist will be formulated, dosed, and administered in a fashion consistent with good medical practice. Factors for consideration in this context include the particular type of RA being treated, the particular mammal being treated, the clinical condition of the individual patient, the cause of the RA, the site of delivery of the antagonist, possible side-effects, the type of antagonist, the method of administration, the scheduling of administration, and other factors known to medical practitioners. The effective amount of the antagonist to be administered will be governed by such considerations.

A physician having ordinary skill in the art can readily determine and prescribe the effective amount of the pharmaceutical composition required, depending on such factors as the particular antagonist type and safety profile. For example, the physician could start with doses of such antagonist, such as an anti-LT alpha antibody, employed in the pharmaceutical composition at levels lower than that required to achieve the desired therapeutic effect to assess safety, and gradually increase the dosage until the desired effect (without compromising safety) is achieved. The effectiveness of a given dose or treatment regimen of the antagonist can be determined, for example, by assessing signs and symptoms in the patient using the standard RA measures of efficacy.

a. Dosage.

For the prevention or treatment of disease, the appropriate dosage of an antibody of the invention (when used alone or in combination with a second medicament as noted below) will depend, for example, on the type of disease to be treated, the type of antibody, the severity and course of the disease, whether the antibody is administered for preventive or therapeutic purposes, previous therapy, the patient's clinical history and response to the antibody, and the discretion of the attending physician. The dosage is preferably efficacious for the treatment of that indication while minimizing toxicity and side effects.

The antibody is suitably administered to the patient at one time or over a series of treatments. Depending on the type and severity of the disease, about 1 μg/kg to 500 mg/kg (preferably about 0.1 mg/kg to 400 mg/kg) of antibody is an initial candidate dosage for administration to the patient, whether, for example, by one or more separate administrations, or by continuous infusion. One typical daily dosage might range from about 1 μg/kg to 500 mg/kg or more, depending on the factors mentioned above. For repeated administrations over several days or longer, depending on the condition, the treatment is sustained until a desired suppression of disease symptoms occurs. One exemplary dosage of the antibody would be in the range from about 0.05 mg/kg to about 400 mg/kg. Thus, one or more doses of about 0.5 mg/kg, 2.0 mg/kg, 4.0 mg/kg or 10 mg/kg or 50 mg/kg or 100 mg/kg or 300 mg/kg or 400 mg/kg (or any combination thereof) may be administered to the patient. Such doses may be administered intermittently, e.g., every week or every three weeks (e.g., such that the patient receives from about two to about twenty, e.g., about six doses of the antibody). An initial higher loading dose, followed by one or more lower doses, may be administered. An exemplary dosing regimen comprises administering an initial loading dose of about 4 to 500 mg/kg, followed by a weekly maintenance dose of about 2 to 400 mg/kg of the antibody. However, other dosage regimens may be useful. The progress of this therapy is easily monitored by conventional techniques and assays.

For the treatment of an autoimmune disorder, the therapeutically effective dosage will typically be in the range of about 50 mg/m² to about 3000 mg/m², preferably about 50 to 1500 mg/m², more preferably about 50-1000 mg/m². In one embodiment, the dosage range is about 125-700 mg/m². For treating RA, in one embodiment, the dosage range for the humanized antibody is about 50 mg/m² or 125 mg/m² (equivalent to about 200 mg/dose) to about 1000 mg/m², given in two doses, e.g., the first dose of about 200 mg is administered on day one followed by a second dose of about 200 mg on day 15. In different embodiments, the dosage is about any one of 50 mg/dose, 80 mg/dose, 100 mg/dose, 125 mg/dose, 150 mg/dose, 200 mg/dose, 250 mg/dose, 275 mg/dose, 300 mg/dose, 325 mg/dose, 350 mg/dose, 375 mg/dose, 400 mg/dose, 425 mg/dose, 450 mg/dose, 475 mg/dose, 500 mg/dose, 525 mg/dose, 550 mg/dose, 575 mg/dose, or 600 mg/dose, or 700 mg/dose, or 800 mg/dose, or 900 mg/dose, or 1000 mg/dose, or 1500 mg/dose.

In treating disease, the LTalpha-binding antibodies of the invention can be administered to the patient chronically or intermittently, as determined by the physician of skill in the disease.

A patient administered a drug by intravenous infusion or subcutaneously may experience adverse events such as fever, chills, burning sensation, asthenia, and headache. To alleviate or minimize such adverse events, the patient may receive an initial conditioning dose(s) of the antibody followed by a therapeutic dose. The conditioning dose(s) will be lower than the therapeutic dose to condition the patient to tolerate higher dosages.

The antibodies herein may be administered at a frequency that is within the skill and judgment of the practicing physician, depending on various factors noted above, for example, the dosing amount. This frequency includes twice a week, three times a week, once a week, bi-weekly, or once a month, In a preferred aspect of this method, the antibody is administered no more than about once every other week, more preferably about once a month.

b. Route of Administration

The antibodies used in the methods of the invention (as well as any second medicaments) are administered to a subject or patient, including a human patient, in accord with suitable methods, such as those known to medical practitioners, depending on many factors, including whether the dosing is acute or chronic. These routes include, for example, parenteral, intravenous administration, e.g., as a bolus or by continuous infusion over a period of time, by subcutaneous, intramuscular, intra-arterial, intraperitoneal, intrapulmonary, intracerebrospinal, intra-articular, intrasynovial, intrathecal, intralesional, or inhalation routes (e.g., intranasal). Parenteral infusions include intramuscular, intravenous, intraarterial, intraperitoneal, or subcutaneous administration. In addition, the antibody is suitably administered by pulse infusion, particularly with declining doses of the antibody. Preferred routes herein are intravenous or subcutaneous administration, most preferably subcutaneous.

In one embodiment, the antibody herein is administered by intravenous infusion, and more preferably with about 0.9 to 20% sodium chloride solution as an infusion vehicle.

As noted above, however, these suggested amounts of antagonist and frequency of dosing are subject to a great deal of therapeutic discretion. The key factor in selecting an appropriate dose and schedule is the result obtained, as indicated above. For example, relatively higher doses may be needed initially for the treatment of ongoing and acute RA. To obtain the most efficacious results, once antagonist therapy is predicted by the biomarkers herein the antagonist is administered as close to the first sign, diagnosis, appearance, or occurrence of the RA as possible or during remissions of the RA.

In all the inventive methods set forth herein, the antagonist (such as an antibody that binds to a LT or a lymphotoxin receptor) may be unconjugated, such as a naked antibody, or may be conjugated with another molecule for further effectiveness, such as, for example, to improve half-life. In one embodiment, the antagonist is a LTα antagonist. In another embodiment, the LT antagonist is an anti-LTα antibody, and more particularly a humanized anti-LTα antibody.

In a further embodiment of the methods herein, the subject has never been previously treated with one or more drugs, such as with a TNF-α inhibitor, e.g., TNFR-Fc or an anti-TNF-α or anti-TNF-α receptor antibody, to treat, for example, RA, or with immunosuppressive agent(s) to treat joint damage or an underlying cause such as an autoimmune disorder, has never been previously treated with a LT antagonist (e.g., an antibody to a LT). In another embodiment, the subject has never been previously treated with an integrin antagonist such as anti-α4 integrin antibody or co-stimulation modulator, an immunosuppressive agent, a cytokine antagonist, an anti-inflammatory agent such as a NSAID, a DMARD other than MTX, except for azathioprine and/or leflunomide, a cell-depleting therapy, including investigational agents (e.g., CAMPATH, anti-CD4, anti-CD5, anti-CD3, anti-CD19, anti-CD11a, anti-CD22, or BLys/BAFF), a live/attenuated vaccine within 28 days prior to baseline, or a corticosteroid such as an intra-articular or parenteral glucocorticoid within 4 weeks prior to baseline. More preferably, the subject has never been treated with an immunosuppressive agent, cytokine antagonist, integrin antagonist, corticosteroid, analgesic, a DMARD, or a NSAID. Still more preferably, the subject has never been treated with an immunosuppressive agent, cytokine antagonist, integrin antagonist, corticosteroid, DMARD, or NSAID.

In a further aspect, the subject may have had a relapse with the RA or joint damage or suffered organ damage such as kidney damage before being treated in any of the methods above, including after the initial or a later antagonist or antibody exposure. However, preferably, the subject has not relapsed with the RA or joint damage and more preferably has not had such a relapse before at least the initial treatment.

In a further embodiment, the subject does not have a malignancy, including a B-cell malignancy, solid tumors, hematologic malignancies, or carcinoma in situ (except basal cell and squamous cell carcinoma of the skin that have been excised and cured). In a still further embodiment, the subject does not have rheumatic autoimmune disease other than RA, or significant systemic involvement secondary to RA (including but not limited to vasculitis, pulmonary fibrosis, or Felty's syndrome). In another embodiment, the subject does have secondary Sjögren's syndrome or secondary limited cutaneous vasculitis. In another embodiment, the subject does not have functional class IV as defined by the ACR Classification of Functional Status in RA. In a further embodiment, the subject does not have inflammatory joint disease other than RA (including, but not limited to, gout, reactive arthritis, psoriatic arthritis, seronegative spondyloarthropathy, or Lyme disease), or other systemic autoimmune disorder (including, but not limited to, SLE, inflammatory bowel disease, scleroderma, inflammatory myopathy, mixed connective tissue disease, or any overlap syndrome). In another embodiment, the subject does not have juvenile idiopathic arthritis (JIA), juvenile RA (JRA), and/or RA before age 16. In another embodiment, the subject does not have significant and/or uncontrolled cardiac or pulmonary disease (including obstructive pulmonary disease), or significant concomitant disease, including but not limited to, nervous system, renal, hepatic, endocrine or gastrointestinal disorders, nor primary or secondary immunodeficiency (history of, or currently active), including known history of HIV infection. In another aspect, the subject does not have any neurological (congenital or acquired), vascular or systemic disorder that could affect any of the efficacy assessments, in particular, joint pain and swelling (e.g., Parkinson's disease, cerebral palsy, or diabetic neuropathy). In a still further embodiment, the subject does not have MS. In a yet further aspect, the subject does not have lupus or Sjögren's syndrome. In still another aspect, the subject does not have an autoimmune disease other than RA. In yet another aspect of the invention, any joint damage in the subject is not associated with an autoimmune disease or with an autoimmune disease other than RA, or with a risk of developing an autoimmune disease or an autoimmune disease other than RA.

For purposes of these lattermost statements, an “autoimmune disease” herein is a disease or disorder arising from and directed against an individual's own tissues or organs or a co-segregate or manifestation thereof or resulting condition therefrom. In many of these autoimmune and inflammatory disorders, a number of clinical and laboratory markers may exist, including, but not limited to, hypergammaglobulinemia, high levels of autoantibodies, antigen-antibody complex deposits in tissues, benefit from corticosteroid or immunosuppressive treatments, and lymphoid cell aggregates in affected tissues. “Autoimmune disease” can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease that can affect multiple organ systems (for example, SLE, RA, polymyositis, etc.). Preferred such diseases include autoimmune rheumatologic disorders (such as, for example, RA, Sjögren's syndrome, scleroderma, lupus such as SLE and lupus nephritis, polymyositis/dermatomyositis, cryoglobulinemia, anti-phospholipid antibody syndrome, and psoriatic arthritis), autoimmune gastrointestinal and liver disorders (such as, for example, inflammatory bowel diseases (e.g., ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-negative vasculitis and ANCA-associated vasculitis, including Churg-Strauss vasculitis, Wegener's granulomatosis, and microscopic polyangiitis), autoimmune neurological disorders (such as, for example, MS, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)). More preferred such diseases include, for example, RA, ulcerative colitis, ANCA-associated vasculitis, lupus, MS, Sjögren's syndrome, Graves' disease, IDDM, pernicious anemia, thyroiditis, and glomerulonephritis.

In another preferred aspect of the above-described method, the subject was administered MTX prior to the baseline or start of treatment. More preferably, the MTX was administered at a dose of about 10-25 mg/week. Also, preferably, the MTX was administered for at least about 12 weeks prior to the baseline, and still more preferably the MTX was administered at a stable dose the last four weeks prior to the baseline. In other embodiments, the MTX was administered perorally or parenterally.

In a particularly preferred embodiment of the above-identified methods, the subject has exhibited an inadequate response to one or more TNF-α inhibitors or to MTX.

In another preferred aspect, MTX is administered to the subject along with the LT antagonist, for example, an anti-LTα antibody.

Also included herein, after the diagnosis step, is a method of monitoring the treatment of bone or soft tissue joint damage in a subject comprising administering an effective amount of a LT antagonist (such as an antibody thereto, including an anti-LTα antibody) to the subject and measuring by imaging techniques such as MRI or radiography after at least about three months, preferably about 24 weeks, from the administration whether the bone or soft tissue joint damage has been reduced over baseline prior to the administration, wherein a decrease versus baseline in the subject after treatment indicates the antagonist such as an anti-LTα antibody is having an effect on the joint damage. Preferably, the degree of reduction versus baseline is measured a second time after the administration of the antagonist such as an antibody or immunoadhesin.

In other aspects, at least about three months after the administration, an imaging test (radiographic and/or MRI) is given that measures a reduction in bone and soft tissue joint damage as compared to baseline prior to the administration, and the amount of antagonist administered is effective in achieving a reduction in the joint damage. Preferably, the test measures a total modified Sharp score. In other preferred embodiments, the method further comprises an additional administration to the patient of a LT antagonist in an amount effective to achieve a continued or maintained reduction in joint damage as compared to the effect of a prior administration of the antagonist. In preferred aspects, the antagonist is additionally administered to the patient even if there is no clinical improvement in the patient at the time of the radiographic testing after a prior administration. Preferably, the clinical improvement is determined by assessing the number of tender or swollen joints, conducting a global clinical assessment of the patient, assessing erythrocyte sedimentation rate, assessing the amount of C-reactive protein level, or using composite measures of disease activity (disease response), such as the DAS-28, ACR-20, -50, or -70 scores.

In yet another aspect, the invention provides, after the diagnosis step, a method of determining whether to continue administering a LT antagonist (such as an anti-LTα antibody) to a subject with bone or soft tissue joint damage comprising measuring reduction in joint damage in the subject, using imaging techniques, such as radiography and/or MRI, after administration of the antagonist a first time, measuring reduction in joint damage in the subject, using imaging techniques such as radiography and/or MRI after administration of the antagonist a second time, comparing imaging findings in the subject at the first time and at the second time, and if the score is less at the second time than at the first time, continuing administration of the antagonist.

In a still further embodiment, a step is included in the treatment method to test for the subject's response to treatment after the administration step to determine that the level of response is effective to treat the bone or soft tissue joint damage. For example, a step is included to test the imaging (radiographic and/or MRI) score after administration and compare it to baseline imaging results obtained before administration to determine if treatment is effective by measuring if, and by how much, it has been changed. This test may be repeated at various scheduled or unscheduled time intervals after the administration to determine maintenance of any partial or complete remission. Alternatively, the methods herein comprise a step of testing the subject, before administration, to see if one or more biomarkers or symptoms are present for joint damage, as set forth above. In another method, a step may be included to check the subject's clinical history, as detailed above, for example, to rule out infections or malignancy as causes, for example, primary causes, of the subject's condition, prior to administering the antagonist to the subject. Preferably, the joint damage is primary (i.e., the leading disease), and is not secondary, such as secondary to infection or malignancy, whether solid or liquid tumors.

In one embodiment of all the methods herein, the antagonist (for example, an anti-LTα antibody) is the only medicament administered to the subject to treat the RA, i.e., no other medicament than the antagonist is administered to the subject to treat the RA.

In any of the methods herein, preferably the antagonist is one of the medicaments used to treat the RA. Thus, one may administer to the subject along with the LT antagonist an effective amount of a second medicament (where the B-LT antagonist (e.g., an anti-LTα antibody) is a first medicament). The second medicament may be one or more medicaments, and includes, for example, an immunosuppressive agent, a cytokine antagonist such as a cytokine antibody, an integrin antagonist (e.g., antibody), a corticosteroid, or any combination thereof. The type of such second medicament depends on various factors, including the type of RA and/or joint damage, the severity of the RA and/or joint damage, the condition and age of the subject, the type and dose of the first medicament employed, etc.

Examples of such additional medicaments include an immunosuppressive agent (such as mitoxantrone (NOVANTRONE®), MTX, cyclophosphamide, chlorambucil, leflunomide, and azathioprine), intravenous immunoglobulin (gamma globulin), lymphocyte-depleting therapy (e.g., mitoxantrone, cyclophosphamide, CAMPATH™ antibodies, anti-CD4, cladribine, a polypeptide construct with at least two domains comprising a de-immunized, autoreactive antigen or its fragment that is specifically recognized by the Ig receptors of autoreactive B-cells (WO 2003/68822), total body irradiation, and bone marrow transplantation), integrin antagonist or antibody (e.g., an LFA-1 antibody such as efalizumab/RAPTIVA® commercially available from Genentech, or an alpha 4 integrin antibody such as natalizumab/ANTEGREN® available from Biogen, or others as noted above), drugs that treat symptoms secondary or related to RA and/or joint damage such as those noted herein, steroids such as corticosteroid (e.g., prednisolone, methylprednisolone such as SOLU-MEDROL™ methylprednisolone sodium succinate for injection, prednisone such as low-dose prednisone, dexamethasone, or glucocorticoid, e.g., via joint injection, including systemic corticosteroid therapy), non-lymphocyte-depleting immunosuppressive therapy (e.g., MMF or cyclosporine), a TNF-α inhibitor such as an antibody to TNF-α or its receptor or TNFR-Fc (e.g., etanercept), DMARD, NSAID, plasmapheresis or plasma exchange, trimethoprim-sulfamethoxazole (BACTRIM™, SEPTRA™), MMF, H2-blockers or proton-pump inhibitors (during the use of potentially ulcerogenic immunosuppressive therapy), levothyroxine, cyclosporin A (e.g., SANDIMMUNE®), somatostatin analogue, a DMARD or NSAID, or a cytokine antagonist such as antibody, anti-metabolite, immunosuppressive agent, rehabilitative surgery, radioiodine, thyroidectomy, or an anti-IL-6 receptor antagonist/antibody (e.g., ACTEMRA™ (tocilizumab)).

Preferred such medicaments include gamma globulin, an integrin antagonist, anti-CD4, cladribine, trimethoprimsulfamethoxazole, an H2-blocker, proton-pump inhibitor, cyclosporine, TNF-α inhibitor, DMARD, NSAID (to treat, for example, musculoskeletal symptoms), levothyroxine, cytokine antagonist (including cytokine-receptor antagonist), anti-metabolite, immunosuppressive agent such as MTX or a corticosteroid, bisphosphonate.

The more preferred such medicaments are an immunosuppressive agent such as MTX or a corticosteroid, a DMARD, an integrin antagonist, a NSAID, a cytokine antagonist, a bisphosphonate, or a combination thereof.

In one particularly preferred embodiment, the second medicament is a DMARD, which is preferably selected from the group consisting of auranofin, chloroquine, D-penicillamine, injectable gold, oral gold, hydroxychloroquine, sulfasalazine, myocrisin, and MTX.

In another such embodiment, the second medicament is a NSAID, which is preferably selected from the group consisting of: fenbufen, naprosyn, diclofenac, etodolac and indomethacin, aspirin, and ibuprofen.

In a further such embodiment, the second medicament is an immunosuppressive agent, which is preferably selected from the group consisting of etanercept, infliximab, adalimumab, leflunomide, anakinra, azathioprine, MTX, and cyclophosphamide.

In other preferred aspects, the second medicament is selected from the group consisting of anti-α4, etanercept, infliximab, etanercept, adalimumab, kinaret, efalizumab, OPG, RANK-Fc, anti-RANKL, pamidronate, alendronate, actonel, zolendronate, clodronate, MTX, azulfidine, hydroxylchloroquine, doxycycline, leflunomide, SSZ, prednisolone, IL-1 receptor antagonist, prednisone, and methylprednisolone.

In still preferred embodiments, the second medicament is selected from the group consisting of infliximab, an infliximab/MTX combination, etanercept, a corticosteroid, cyclosporin A, azathioprine, auranofin, hydroxychloroquine (HCQ), a combination of prednisolone, MTX, and SSZ, a combination of MTX, SSZ, and HCQ, a combination of cyclophosphamide, azathioprine, and HCQ, and a combination of adalimumab with MTX. If the second medicament is a corticosteroid, preferably it is prednisone, prednisolone, methylprednisolone, hydrocortisone, or dexamethasone. Also, preferably, the corticosteroid is administered in lower amounts than are used if the antagonist is not administered to a subject treated with a corticosteroid as standard-of-care therapy. Most preferably, the second medicament is MTX.

All these second medicaments may be used in combination with each other or by themselves with the first medicament, so that the expression “second medicament” as used herein does not mean it is the only medicament besides the first medicament, respectively. Thus, the second medicament need not be one medicament, but may constitute or comprise more than one such drug.

These second medicaments as set forth herein are generally used in the same dosages and with administration routes as used hereinbefore or about from 1 to 99% of the heretofore-employed dosages. If such second medicaments are used at all, preferably, they are used in lower amounts than if the first medicament were not present, especially in subsequent dosings beyond the initial dosing with the first medicament, so as to eliminate or reduce side effects caused thereby.

The combined administration of a second medicament includes co-administration (concurrent administration), using separate formulations or a single pharmaceutical formulation, and consecutive administration in either order, wherein preferably there is a time period while both (or all) active agents (medicaments) simultaneously exert their biological activities.

The LT antagonists described herein are administered by any suitable means, including parenteral, topical, intraperitoneal, intrapulmonary, intranasal, and/or intralesional administration. Parenteral infusions include intramuscular, intravenous (i.v.), intraarterial, intraperitoneal, or subcutaneous (s.c.) administration. Intrathecal administration is also suitable. Also the antagonist may suitably be administered by pulse infusion, e.g., with declining doses of the antagonist. Preferably if the antagonist is an antibody, the dosing is given by i.v. or s.c. means, and more preferably by i.v. infusion(s) or injection(s).

Aside from administration of antagonists to the patient by traditional routes as noted above, the present invention includes administration by gene therapy. Such administration of nucleic acids encoding the antagonist is encompassed by the expression “administering an effective amount of an antagonist”. See, for example, WO 1996/07321 concerning the use of gene therapy to generate intracellular antibodies.

There are two major approaches to getting the nucleic acid (optionally contained in a vector) into the patient's cells, in vivo and ex vivo. For in vivo delivery the nucleic acid is injected directly into the patient, usually at the site where the antagonist is required. For ex vivo treatment, the patient's cells are removed, the nucleic acid is introduced into these isolated cells, and the modified cells are administered to the patient either directly or, for example, encapsulated within porous membranes that are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent specific for the target cells, such as an antibody specific for a cell-surface membrane protein on the target cell, a ligand for a receptor on the target cell, etc. Where liposomes are employed, proteins that bind to a cell-surface membrane protein associated with endocytosis may be used for targeting and/or to facilitate uptake, e.g. capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem., 262:4429-4432 (1987) and Wagner et al., Proc. Natl. Acad. Sci. USA, 87:3410-3414 (1990). Gene-marking and gene-therapy protocols are described, for example, in Anderson et al., Science, 256:808-813 (1992) and WO 1993/25673.

In another embodiment, a method is provided for treating joint damage in a subject eligible for treatment based on the biomarker analysis herein comprising administering a LT antagonist, such as an antibody thereto, for example, anti-LTα antibody, to the subject, and giving the subject, at least about 52 weeks after the administration, an imaging test that measures a reduction in the joint damage as compared to baseline prior to the administration, wherein the amount of antagonist such as an anti-LTα antibody administered is effective in achieving a reduction in the joint damage, indicating that the subject has been successfully treated.

In this method, preferably the test measures a total modified Sharp score. In another preferred embodiment of this joint-treatment method, the antagonist is an anti-LTα antibody.

In another preferred embodiment, the joint damage is caused by arthritis, preferably RA, and more preferably early or incipient RA. In all the methods herein, the RA is preferably early or incipient RA. The subject herein may be RF negative or positive.

In another aspect, such method further comprises re-treating the subject by providing an additional administration to the subject of the antagonist such as an anti-LTα antibody in an amount effective to treat RA or achieve a continued or maintained reduction in joint damage as compared to the effect of a prior administration of the antagonist. The re-treatment may be commenced at least about 24 weeks (preferably at about 24 weeks) after the first administration of the antagonist, and one or more further re-treatments is optionally commenced. In another embodiment, the further re-treatment is commenced at least about 24 weeks after the second administration of the antagonist.

In one aspect the antagonist is additionally administered to the subject even if there is no clinical improvement in the subject at the time of RA testing or another imaging testing after a prior administration.

In a further preferred aspect, RA or joint damage has been reduced after the re-treatment as compared to the extent of RA or joint damage after the first assessment such as imaging assessment.

If multiple exposures of antagonist are provided as in re-treatment, each exposure may be provided using the same or a different administration means. In one embodiment, each exposure is by i.v. administration. In another embodiment, each exposure is given by s.c. administration. In yet another embodiment, the exposures are given by both i.v. and s.c. administration.

Preferably the same antagonist, such as an anti-LTα antibody is used for at least two antagonist exposures, and preferably for each antagonist exposure. Thus, the initial and second antagonist exposures are preferably with the same antagonist, and more preferably all antagonist exposures are with the same antagonist, i.e., treatment for the first two exposures, and preferably all exposures, is with one type of LT antagonist, e.g., an antagonist that binds to a LT, such as an anti-LTα antibody.

Preferably, in this re-treatment method, a second medicament is administered in an effective amount, wherein the antagonist is a first medicament. In one aspect, the second medicament is more than one medicament. In another aspect, the second medicament is one of those set forth above, including an immunosuppressive agent, a DMARD, an integrin antagonist, a NSAID, a cytokine antagonist, a bisphosphonate, or a combination thereof, most preferably MTX.

For the re-treatment methods described herein, where a second medicament is administered in an effective amount with an antagonist exposure, it may be administered with any exposure, for example, only with one exposure, or with more than one exposure. In one embodiment, the second medicament is administered with the initial exposure. In another embodiment, the second medicament is administered with the initial and second exposures. In a still further embodiment, the second medicament is administered with all exposures. It is preferred that after the initial exposure, such as of steroid, the amount of such second medicament is reduced or eliminated so as to reduce the exposure of the subject to an agent with side effects such as prednisone, prednisolone, methylprednisolone, and cyclophosphamide.

In one embodiment of the re-treatment method, the subject has never been previously administered any drug(s), such as immunosuppressive agent(s), to treat the RA or joint damage. In another aspect, the subject or patient is responsive to previous therapy for the RA or joint damage.

In another aspect of re-treatment, the subject or patient has been previously administered one or more medicaments(s) to treat the RA or joint damage. In a further embodiment, the subject or patient was not responsive to one or more of the medicaments that had been previously administered. Such drugs to which the subject may be non-responsive include, for example, chemotherapeutic agents, immunosuppressive agents, cytokine antagonists, integrin antagonists, corticosteroids, analgesics, or LT antagonists such as antagonists to a LT or a lymphotoxin receptor, for example, an anti-LTα antibody. More particularly, the drugs to which the subject may be non-responsive include immunosuppressive agents or LT antagonists such as an anti-LTα antibodies. Preferably, such antagonists are not antibodies or immunoadhesins, and are, for example, small-molecule inhibitors, or anti-sense oligonucleotides, or antagonistic peptides, as noted, for example, in the background section. In a further aspect, such antagonists include an antibody or immunoadhesin, such that re-treatment is contemplated with one or more antibodies or immunoadhesins of this invention to which the subject was formerly non-responsive. Most preferably, the subject or patient is not responsive to previous therapy with MTX or a TNF-α inhibitor.

In another embodiment, a method is provided for treating joint damage in a subject comprising administering a LT antagonist, such as an antibody thereto, for example, an anti-LTα antibody, to the subject, and giving the subject, at least about 52 weeks after the administration, an imaging test that measures a reduction in the joint damage as compared to baseline prior to the administration, wherein the amount of LT antagonist administered is effective in achieving a reduction in the joint damage, indicating that the subject has been successfully treated.

In this method, preferably the test measures a total modified Sharp score. In another preferred embodiment of this joint-treatment method, the antagonist is an anti-LTα antibody.

Preferably, in this method regarding the about 52-week assessment, a second medicament is administered in an effective amount, wherein the antagonist such as anti-LTα antibody is a first medicament. In one aspect, the second medicament is more than one medicament. In another aspect, the second medicament is one of those set forth above, including an immunosuppressive agent, a DMARD, an integrin antagonist, a NSAID, a cytokine antagonist, a bisphosphonate, or a combination thereof, most preferably MTX.

In a further aspect, the invention involves a method of reducing the risk of a negative side effect in a subject (e.g., selected from the group consisting of an infection, cancer, heart failure, and demyelination) comprising administering to the subject an effective amount of a LT antagonist if the subject has one or more of the biomarkers herein.

A discussion of methods of producing, modifying, and formulating such antagonists follows.

E. Production of Antagonists

The methods and articles of manufacture of the present invention use, or incorporate, a LT antagonist such as an antibody. Methods for screening for such antagonists are noted above. Methods for generating such antagonists are well within the skill of the art, and include chemical synthesis, recombinant production, hybridoma production, peptide synthesis, oligonucleotide synthesis, phage-display, etc., depending on the type of antagonist being produced.

While the preferred antagonist is an antibody, other antagonists are contemplated herein. For example, the antagonist may comprise a small-molecule antagonist optionally fused to, or conjugated with, a cytotoxic agent. Libraries of small molecules may be screened against LT antigens of interest herein to identify a small molecule that binds to that antigen. The small molecule may further be screened for its antagonistic properties and/or conjugated with a cytotoxic agent.

The antagonist may also be a peptide generated by rational design or by phage display (see, e.g., WO 1998/35036). In one embodiment, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide.

A description follows as to exemplary techniques for the production of the antibody antagonists used in accordance with the present invention.

a. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C=NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with 1/5 to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

b. Monoclonal Antibodies

Monoclonal antibodies are obtained from a population of substantially homogeneous antibodies, i.e., the individual antibodies comprising the population are identical and/or bind the same epitope except for possible variants that arise during production of the monoclonal antibody, such variants generally being present in minor amounts. Thus, the modifier “monoclonal” indicates the character of the antibody as not being a mixture of discrete or polyclonal antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

c. Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

d. Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807.

Alternatively, phage display technology (McCafferty et al., Nature, 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson and Chiswell, Current Opinion in Structural Biology, 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol., 222:581-597 (1991), or Griffith et al., EMBO J., 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

e. Antibody Fragments

Various techniques have been developed for the production of antibody fragments. Traditionally, these fragments were derived via proteolytic digestion of intact antibodies (see, e.g., Morimoto et al., J. Biochem. Biophys. Methods, 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology, 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 1993/16185; U.S. Pat. No. 5,571,894; and U.S. Pat. No. 5,587,458. The antibody fragment may also be a “linear antibody”, e.g., as described in U.S. Pat. No. 5,641,870 for example. Such linear antibody fragments may be monospecific or bispecific.

f. Bispecific Antibodies

Bispecific antibodies are antibodies that have binding specificities for at least two different epitopes. Exemplary bispecific antibodies may bind to two different epitopes of a LTα antigen. Bispecific antibodies can be prepared as full-length antibodies or antibody fragments (e.g. F(ab′)₂ bispecific antibodies).

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 1993/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 1994/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 1991/00360, WO 1992/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol., 147:60 (1991).

F. Modifications of the Antagonist

Modifications of the antagonist are contemplated herein. For example, the antagonist may be linked to one of a variety of nonproteinaceous polymers, e.g., polyethylene glycol (PEG), polypropylene glycol, polyoxyalkylenes, or copolymers of polyethylene glycol and polypropylene glycol. Antibody fragments, such as Fab′, linked to one or more PEG molecules are a therapeutic embodiment of the invention.

The antagonists disclosed herein may also be formulated as liposomes. Liposomes containing the antagonist are prepared by methods known in the art, such as described in Epstein et al., Proc. Natl. Acad. Sci. USA, 82:3688 (1985); Hwang et al., Proc. Natl. Acad. Sci. USA, 77:4030 (1980); U.S. Pat. Nos. 4,485,045 and 4,544,545; and WO 1997/38731. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al., J. Biol. Chem., 257:286-288 (1982) via a disulfide interchange reaction. A chemotherapeutic agent is optionally contained within the liposome. See Gabizon et al., J. National Cancer Inst., 81(19):1484 (1989).

Amino acid sequence modification(s) of protein or peptide antagonists described herein are contemplated. For example, it may be desirable to improve the binding affinity and/or other biological properties of the antagonist. Amino acid sequence variants of the antagonist are prepared by introducing appropriate nucleotide changes into the antagonist nucleic acid, or by peptide synthesis. Such modifications include, for example, deletions from, and/or insertions into and/or substitutions of, residues within the amino acid sequences of the antagonist. Any combination of deletion, insertion, and substitution is made to arrive at the final construct, provided that the final construct possesses the desired characteristics. The amino acid changes also may alter post-translational processes of the antagonist, such as changing the number or position of glycosylation sites.

A useful method for identification of certain residues or regions of the antagonist that are preferred locations for mutagenesis is called “alanine scanning mutagenesis” as described by Cunningham and Wells, Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antagonist variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antagonist with an N-terminal methionyl residue or the antagonist fused to a cytotoxic polypeptide. Other insertional variants of the antagonist molecule include the fusion to the N- or C-terminus of the antagonist of an enzyme, or a polypeptide which increases the serum half-life of the antagonist.

Another type of variant is an amino acid substitution variant. These variants have at least one amino acid residue in the antagonist molecule replaced by different residue. The sites of greatest interest for substitutional mutagenesis of antibody antagonists include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1

| Original | Exemplary | Preferred |
|---|---|---|
| Residue | Substitutions | Substitutions |
| Ala (A) | val; leu; ile | Val |
| Arg (R) | lys; gln; asn | Lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | Ala | ala |
| His (H) | asn; gln; lys; arg | arg |
| Ile (I) | leu; val; met; ala; | leu |
| | phe; norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | tyr |
| Pro (P) | Ala | ala |
| Ser (S) | Thr | thr |
| Thr (T) | Ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in the biological properties of the antagonist are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

hydrophobic: norleucine, met, ala, val, leu, ile;

neutral hydrophilic: cys, ser, thr;

acidic: asp, glu;

basic: asn, gln, his, lys, arg;

residues that influence chain orientation: gly, pro; and

aromatic: trp, tyr, phe.

Non-conservative substitutions will entail exchanging a member of one of these classes for another class.

Any cysteine residue not involved in maintaining the proper conformation of the antagonist also may be substituted, generally with serine, to improve the oxidative stability of the molecule and prevent aberrant crosslinking Conversely, cysteine bond(s) may be added to the antagonist to improve its stability (particularly where the antagonist is an antibody fragment such as an Fv fragment).

A particularly preferred type of substitutional variant involves substituting one or more HVR residues of a parent antibody. Generally, the resulting variant(s) selected for further development will have improved biological properties relative to the parent antibody from which they are generated. A convenient way for generating such substitutional variants is affinity maturation using phage display. Briefly, several HVR sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. Alanine-scanning mutagenesis can be performed to identify candidate HVR residues contributing significantly to antigen binding for possible modification. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antagonist alters the original glycosylation pattern of the antagonist. Such altering includes deleting one or more carbohydrate moieties found in the antagonist, and/or adding one or more glycosylation sites that are not present in the antagonist.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antagonist is typically accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antagonist (for O-linked glycosylation sites).

Where the antibody comprises an Fc region, the carbohydrate attached thereto may be altered. For example, antibodies with a mature carbohydrate structure that lacks fucose attached to an Fc region of the antibody are described in US 2003/0157108 (Presta). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju) and WO 1999/22764 (Raju) concerning antibodies with altered carbohydrate attached to the Fc region thereof. See also US 2005/0123546 (Umana et al.); US 2004/0072290 (Umana et al.); US 2003/0175884 (Umana et al.); and WO 2005/044859 (Umana et al.) on antigen-binding molecules with modified glycosylation, including antibodies with an Fc region containing N-linked oligosaccharides.

The preferred glycosylation variant herein comprises an Fc region, wherein a carbohydrate structure attached to the Fc region lacks fucose. Such variants have improved ADCC function. Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; US 2006/0063254; US 2006/0064781; US 2006/0078990; US 2006/0078991; Okazaki et al., J. Mol. Biol., 336:1239-1249 (2004); Yamane-Ohnuki et al., Biotech. Bioeng., 87:614 (2004). Examples of cell lines producing defucosylated antibodies include Lec13 CHO cells deficient in protein fucosylation (Ripka et al., Arch. Biochem. Biophys., 249:533-545 (1986); US 2003/0157108 A1 (Presta) and WO 2004/056312 A1 (Adams et al., especially at Example 11), and knockout cell lines, such as alpha-1,6-fucosyltransferase gene, FUT8, knockout CHO cells (Yamane-Ohnuki et al., Biotech. Bioeng., 87:614 (2004)). See also Kanda et al., Biotechnol. Bioeng., 94:680-688 (2006). US 2007/0048300 (Biogen-IDEC) discloses a method of producing aglycosylated Fc-containing polypeptides, such as antibodies, having desired effector function, as well as aglycosylated antibodies produced according to the method and as methods of using such antibodies as therapeutics.

See also US 2006/024304 (Gerngross et al.); U.S. Pat. No. 7,029,872 (Gerngross); US 2004/018590 (Gerngross et al.); US 2006/034828 (Gerngross et al.); US 2006/034830 (Gerngross et al.); US 2006/029604 (Gerngross et al.); WO 2006/014679 (Gerngross et al.); WO 2006/014683 (Gerngross et al.); WO 2006/014685 (Gerngross et al.); WO 2006/014725 (Gerngross et al.); and WO 2006/014726 (Gerngross et al.) on recombinant glycoproteins and glycosylation variants.

Nucleic acid molecules encoding amino-acid-sequence variants of the antagonist are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antagonist.

To increase the serum half life of the antagonist, one may incorporate a salvage receptor binding epitope into the antagonist (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Antibodies with substitutions in an Fc region thereof and increased serum half-lives are also described in WO 2000/42072 (Presta, L.).

Wngineered antibodies with three or more (preferably four) functional antigen binding sites are also contemplated (US 2002/0004587, Miller et al.).

G. Pharmaceutical Formulations

Therapeutic formulations of the antagonists used in accordance with the present invention are prepared for storage by mixing the antagonist having the desired degree of purity with optional pharmaceutically acceptable carriers, excipients, or stabilizers in the form of lyophilized formulations or aqueous solutions. For general information concerning formulations, see, e.g., Gilman et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Eastori, Pa.; Avis et al., (eds.) (1993) Pharmaceutical Dosage Forms: Parenteral Medications Dekker, New York; Lieberman et al., (eds.) (1990) Pharmaceutical Dosage Forms: Tablets Dekker, New York; and Lieberman et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™, or polyethylene glycol (PEG).

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

Crystallized forms of the antagonist are also contemplated. See, for example, US 2002/0136719A1 (Shenoy et al.).

The formulation herein may also contain more than one active compound (a second medicament as noted above), preferably those with complementary activities that do not adversely affect each other. The type and effective amounts of such medicaments depend, for example, on the amount and type of LT antagonist present in the formulation, and clinical parameters of the subjects. The preferred such second medicaments are noted above.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

H. Articles of Manufacture

Articles of manufacture containing materials useful for the treatment of the RA described above are provided herein. The article of manufacture comprises a container and a label or package insert on or associated with the container. In this aspect, the package insert is on or associated with the container. Suitable containers include, for example, bottles, vials, syringes, etc. The containers may be formed from a variety of materials such as glass or plastic. The container holds or contains the antagonist that is effective for treating the RA or joint damage and may have a sterile access port (for example, the container may be an intravenous solution bag or a vial having a stopper pierceable by a hypodermic injection needle). At least one active agent in the composition is the LT antagonist. The label or package insert indicates that the composition is used for treating joint damage or RA in a subject eligible for treatment with specific guidance regarding dosing amounts and intervals of antagonist and any other medicament being provided.

The article of manufacture may further comprise a second container comprising a pharmaceutically acceptable diluent buffer, such as bacteriostatic water for injection (BWFI), phosphate-buffered saline, Ringer's solution, and dextrose solution. The article of manufacture may further include other materials desirable from a commercial and user standpoint, including other buffers, diluents, filters, needles, and syringes.

The kits and articles of manufacture of the present invention also include information, for example in the form of a package insert or label, indicating that the composition is used for treating RA or joint damage where levels of one or more of the three cytokines herein no greater than predetermined threshold levels for each cytokine are detected in a serum sample from the patient with the disease. The insert or label may take any form, such as paper or electronic media, for example, a magnetically recorded medium (e.g., floppy disk) or a CD-ROM. The label or insert may also include other information concerning the pharmaceutical compositions and dosage forms in the kit or article of manufacture.

Generally, such information aids patients and physicians in using the enclosed pharmaceutical compositions and dosage forms effectively and safely. For example, the following information regarding the antagonist may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

In a preferred embodiment the article of manufacture herein further comprises a container comprising a second medicament, wherein the antagonist is a first medicament, and which article further comprises instructions on the package insert for treating the patient with the second medicament, in an effective amount. The second medicament may be any of those set forth above, with an exemplary second medicament being those set forth above, including an immunosuppressive agent, a corticosteroid, a DMARD, an integrin antagonist, a NSAID, a cytokine antagonist, a bisphosphonate, or a combination thereof, more preferably a DMARD, NSAID, cytokine antagonist, integrin antagonist, or immunosuppressive agent. Most preferably, the second medicament is MTX.

In another aspect, the invention provides a method for manufacturing a LT antagonist or a pharmaceutical composition thereof comprising combining in a package the antagonist or pharmaceutical composition and a label stating that the antagonist or pharmaceutical composition is indicated for treating patients with RA from whom sample(s) has/have been obtained showing levels of one or more of the biomarkers herein no greater than predetermined threshold levels for each biomarker by assessing the levels of one or more of the biomarkers. This can be alone or in combination with showing the presence or amounts of other biomarkers in the sample. The same method can apply to joint damage.

The present invention further provides a method for treating RA in a patient comprising administering to the patient an effective amount of an anti-arthritis therapy other than a LT antagonist, such as a DMARD (including MTX), or cytokine- or integrin-directed biologic therapy, wherein a sample from the patient before administration of the therapy exhibits a level of one or more of the biomarkers described herein greater than predetermined threshold levels for each as defined herein. The sample may be assessed, for example, by any of the methods described herein for determining the levels of one or more of such biomarkers. The assessment of the sample identifies the patient as one who is less likely or not likely to demonstrate an effective response to treatment with a LT antagonist.

I. Methods of Advertising

Advertising is generally paid communication through a non-personal medium in which the sponsor is identified and the message is controlled. Advertising for purposes herein includes publicity, public relations, product placement, sponsorship, underwriting, and sales promotion. This term also includes sponsored informational public notices appearing in any of the print communications media designed to appeal to a mass audience to persuade, inform, promote, motivate, or otherwise modify behavior toward a favorable pattern of purchasing, supporting, or approving the invention herein.

The advertising and promotion of the diagnostic method herein may be accomplished by any means. Examples of advertising media used to deliver these messages include television, radio, movies, magazines, newspapers, the internet, and billboards, including commercials, which are messages appearing in the broadcast media. Advertisements also include those on the seats of grocery carts, on the walls of an airport walkway, and on the sides of buses, or heard in telephone hold messages or in-store PA systems, or anywhere a visual or audible communication can be placed. More specific examples of promotion or advertising means include television, radio, movies, the internet such as webcasts and webinars, interactive computer networks intended to reach simultaneous users, fixed or electronic billboards and other public signs, posters, traditional or electronic literature such as magazines and newspapers, other media outlets, presentations or individual contacts by, e.g., e-mail, phone, instant message, postal, courier, mass, or carrier mail, in-person visits, etc.

The type of advertising used will depend on many factors, for example, on the nature of the target audience to be reached, e.g., hospitals, insurance companies, clinics, doctors, nurses, and patients, as well as cost considerations and the relevant jurisdictional laws and regulations governing advertising of medicaments and diagnostics. The advertising may be individualized or customized based on user characterizations defined by service interaction and/or other data such as user demographics and geographical location.

Many alternative experimental methods known in the art may be successfully substituted for those specifically described herein in the practice of this invention, as for example described in many of the excellent manuals and textbooks available in the areas of technology relevant to this invention (e.g. Using Antibodies, A Laboratory Manual, edited by Harlow, E. and Lane, D., 1999, Cold Spring Harbor Laboratory Press, (e.g. ISBN 0-87969-544-7); Roe B. A. et. al. 1996, DNA Isolation and Sequencing (Essential Techniques Series), John Wiley & Sons (e.g. ISBN 0-471-97324-0); Methods in Enzymology: Chimeric Genes and Proteins, 2000, ed. J. Abelson, M. Simon, S. Emr, J. Thomer. Academic Press; Molecular Cloning: a Laboratory Manual, 2001, 3rd Edition, by Joseph Sambrook and Peter MacCallum, (the former Maniatis Cloning manual) (e.g. ISBN 0-87969-577-3); Current Protocols in Molecular Biology, Ed. Fred M. Ausubel, et. al. John Wiley & Sons (e.g. ISBN 0-471-50338-X); Current Protocols in Protein Science, Ed. John E. Coligan, John Wiley & Sons (e.g. ISBN 0-471-11184-8); and Methods in Enzymology: Guide to protein Purification, 1990, Vol. 182, Ed. Deutscher, M. P., Academic Press, Inc. (e.g. ISBN 0-12-213585-7)), or as described in the many university and commercial websites devoted to describing experimental methods in molecular biology.

Further details of the invention are illustrated by the following non-limiting Examples. The disclosures of all citations in the specification are expressly incorporated herein by reference.

### IV. Examples

The following examples show for the first time the cleavage of LTαβ heterotrimers from the cell membrane and release of the cleaved, solLTαβ into circulation, and increased levels of the solLTαβ found in RA synovial fluid. Also described herein is the ability of solLTαβ to activate fibroblast-like synoviocytes (FLS) isolated from RA patients.

Lymphotoxin (LT) is a TNF superfamily member and is secreted from activated lymphocytes as a trimeric cytokine (LTα3) or complexed on the cell-surface with transmembrane bound LTβ predominantly as LTα1β2. The present invention provides a specific assay for human LTαβ that detects both LTα1β2 and LTα2β1 heterotrimers, but not LTα3. Using this assay, the present inventors show here that surface LTαβ complexes are shed from the surface of activated human polarized Th1 cells. The mechanism is partially dependent on matrix metalloproteinases, as a TACE (TNFα convertase enzyme) inhibitor reduced soluble LTαβ levels shed into the culture fluid in the absence of affecting cell surface or mRNA expression. Circulating levels of solLTαβ were detected in serum from normal donors. SolLTαβ was also detected in serum from RA patients and in synovial fluid taken from diseased joints. SolLTαβ levels found in serum were similar between healthy donors and RA patients, however, synovial fluid from RA joints had significantly higher levels of solLTαβ than synovial fluid from patients with osteoarthritis. In addition, solLTαβ activated primary synovial fibroblasts.

Soluble LTαβ may act as a proinflammatory mediator and be a relevant biomarker in RA patients.

### Example 1

### Assays

This example describes electrochemiluminescent assays (ECLA) specific for human LTα3 and LTαβ. Other assays used in subsequent Examples are also described.

Human LTβR-Fc was constructed as follows: human LTβR encompassing the extracellular domain (position 1 through position 224) was cloned into a modified pRK5 expression vector encoding the human IgG1 Fc region downstream of the LTβR sequence. Proteins were overexpressed in CHO cells and purified by protein A affinity chromatography, as previously described (Grogan/Spits manuscript in press Nature Immunology).

Murine LTβR-Fc was constructed as follows: murine LTβR encompassing the extracellular domain (position 1 through position 222) was cloned into a modified pRK5 expression vector encoding the murine IgG2a Fc region downstream of the LTβR sequence. Proteins were overexpressed in CHO cells and purified by protein A affinity chromatography.

### A. Mouse LT Assays

### Electrochemiluminescent Assay for Measurement of Murine LTα3

Soluble mouse LTα3 was measured by coating a High Bind 96-Well Microtiter plate (Meso Scale Discovery) with 35 μg/ml of goat anti-mouse LTα Clone AF749 (R&D Systems) diluted in PBS/0.05% Tween® 20 for one hour. The wells were then blocked with 150 uL PBS containing 5% bovine serum albumin for 1-2 hours. The wells were washed 6× with PBS containing 0.05% Tween®-20 (PBS/Tween®) and a titration curve of recombinant mouse LTα3 (R&D Systems), controls and unknown test samples diluted in assay diluent (AD: PBS, 0.5% BSA, 0.05% Tween 20, 10 ppm Proclin) were added at 25 μl/well and incubated for 2 hours. The wells were washed 6× with PBS/Tween® and an anti-LTα monoclonal antibody (N3EV) (Genentech) labeled with SULFO-TAG® NHS (Meso Scale Discovery) was diluted to 3 ug/ml in assay diluent and added to the wells at 25 μl/well for 1 hour. The plate was washed with wash buffer 6× and 150 μl/well of Read Buffer T (Meso Scale Discovery) diluted 1:2 in H₂0 was added to the plate. The plate was immediately read on an MA6000 SECTOR™ Imager (Meso Scale Discovery). A weighted 4-parameter fit curve was plotted using XLFit (Guildford, UK) from the resulting standard curve values and unknown concentrations were extrapolated.

### Electrochemiluminescent Assay for Measurement of Murine LTαβ

Soluble mouse LTαβ was measured using streptavidin coated 96-Well Microtiter plates (Meso Scale Discovery). Wells were then blocked with 150 uL of PBS containing 5% bovine serum albumin for 1-2 hours. Twenty-five uL per well of 1 μg/ml murine LTβR-Fc-biotin (R&D Systems) diluted in PBS/0.05% Tween® was then incubated with the blocked plate for 30 minutes. The wells were washed 6× with PBS containing 0.05% Tween®-20 (PBS/Tween®) and a titration curve of recombinant mouse LTα1β2 (R&D Systems), controls and unknown test samples diluted in high salt assay diluent (PBS, 0.5% BSA, 0.05% Tween® 20, 10 ppm Proclin, 5 mM EDTA, 0.2% BGG, 0.25% CHAPS+3.5 mM NaCl pH 7.4) were added at 25 μl/well and incubated for 2 hours. The wells were washed 6× with PBS/Tween® and an anti-LTα monoclonal antibody (N3EV) (Genentech) labeled with SULFO-TAG® NHS (Meso Scale Discovery) was diluted to 3 ug/ml in assay diluent and added to the wells at 25 μl/well for 1 hour. The plate was washed with wash buffer 6× and 150 μl/well of Read Buffer T (Meso Scale Discovery) diluted 1:2 in H₂0 was added to the plate. The plate was immediately read on an MA6000 SECTOR™ Imager (Meso Scale Discovery). A weighted 4-parameter fit curve was plotted using XLFit (Guildford, UK) from the resulting standard curve values and unknown concentrations were extrapolated.

### B. Human LT Assays

### Electrochemiluminescent Assay for Measurement of Human LTα3

A human LTα3 standard was generated in house as follows: The coding sequence of the extra-cellular domain of human LTα (P. W. Gray et al., 1984, Nature 312:721-724), was fused downstream of the trp promoter and ribosome binding site (D G Yansura and D J Henner, 1990, Methods in Enzymology 185:54-60) in the plasmid pBR322 (J G Sutcliffe, 1978, Cold Spring Harbor Symposium Quant. Biol. 43:77) to create an expression construct for this protein in E. coli. Small scale protein inductions were carried out in shake flasks by diluting overnight LB cultures (20×) into either M9 minimal media (J Sambrook et al., 1989, Molecular Cloning: A labatory Manual, Cold Spring Harbor Laboratory, Cold Spring Harbor, N.Y.) or (100×) into CRAP media (L C Simmons et al., 2002, J. Immunol. Methods 263:133-147). Inductions were then allowed to proceed overnight at 30° C. with shaking Samples were removed the next day for expression analysis by SDS PAGE while the bulk of the cell paste was centrifuged and frozen prior to purification. E coli paste expressing LTα3 was extracted by microfluidization and the LTα3 purified by a combination of ion exchange and gel filtration steps as described alpha (P. W. Gray et al., 1984, Nature 312:721-724).

High Bind 96-well microtiter plates (Meso Scale Discovery (MSD), Gaithersburg, Md.) were spotted with 5 uL goat anti-human LTα clone AF211 (R&D Systems) diluted in PBS/0.05% Tween 20 (PBS/Tween) to 2 μg/ml, and incubated for one hour at room temperature. Wells were blocked with 150 uL PBS+5% BSA for 1-2 hours with agitation. Plates were washed 6× with PBS/Tween and a titration curve of recombinant human LTα3 (Genentech Inc.) controls, and test samples diluted in high salt assay diluent (HSAD: PBS, 0.5% BSA, 0.05% Tween 20, 0.25% CHAPS, 5 mM EDTA, 0.20% BgG, 0.35M NaCl, 10% FBS) were added at 25 μl/well and incubated for 2 hours with agitation. Plates were washed 6× with PBS/Tween, an anti-LTα biotinylated PAb (BAF-211, R&D Systems) diluted to 2 ug/ml in AD was added at 25 μL/well, and plates were incubated 1 hour with agitation. Plates were washed, 500 ng/mL Strepavidin-Sulgo-TAG® (MSD) diluted in AD was added at 25 μL/well, and plates were incubated for 30 minutes with agitation. Plates were washed with PBS/Tween 6×, and 150 μl/well of Read Buffer T (MSD) diluted 1:2 in H₂0 was added to wells. Plates were read on an MA6000 Sector™ Imager (MSD) according to manufacturer's. A weighted 4-parameter fit standard curve was plotted using XLFit (Guildford, UK) and values of unknowns were extrapolated.

An alternative protocol is as follows: High Bind 96-well microtiter plates (MSD), were coated with 4 ug/mL goat anti-human LTα AF211 in PBS/0.05% Tween 20 (Sigma) and blocked with Human Serum Cytokine Assay Diluent (SCAD) (MSD). Plates were then processed as above.

### Electrochemiluminescent Assay for Measurement of Human LTαβ

High Bind 96-well microtiter plates (MSD) were spotted with 5 uL of 25 μg/ml recombinant human LTβR-Fc fusion protein (Genentech Inc.) diluted in PBS/Tween, and incubated 1 hour at RT. Wells were blocked and washed as above, a titration curve of recombinant human LTα1β2 (R&D Systems), controls, and test samples diluted in AD were added at 25 μl/well, ands plates were incubated 2 hours with agitation. Plates were washed as above, an anti-LTα biotinylated PAb (BAF-211, R&D Systems) diluted to 1 ug/ml in AD was added to the wells at 25 μL/well, and plates were incubated 1 hour with agitation. Plates were washed and incubated with Strepavidin-Sulfo-TAG® (Meso Scale Discovery), developed with MSD read buffer, read on a MA6000 Sector™ Imager (Meso Scale Discovery), and plotted as above.

### C. TNFα Assay

### Electrochemiluminescent Assay for Measurement of Human TNF-α

Soluble human TNF-α, was detected using a human 96-well TNF-α kit (K111BHA-4, Meso Scale Discovery) according to manufacturer's instructions.

### Electrochemiluminescent Assay for Measurement of Murine TNF-α

Soluble murine TNF-α, was quantified using 96-well muTNF-α kit (K112BHA-4, MSD) according to manufacturer's instructions.

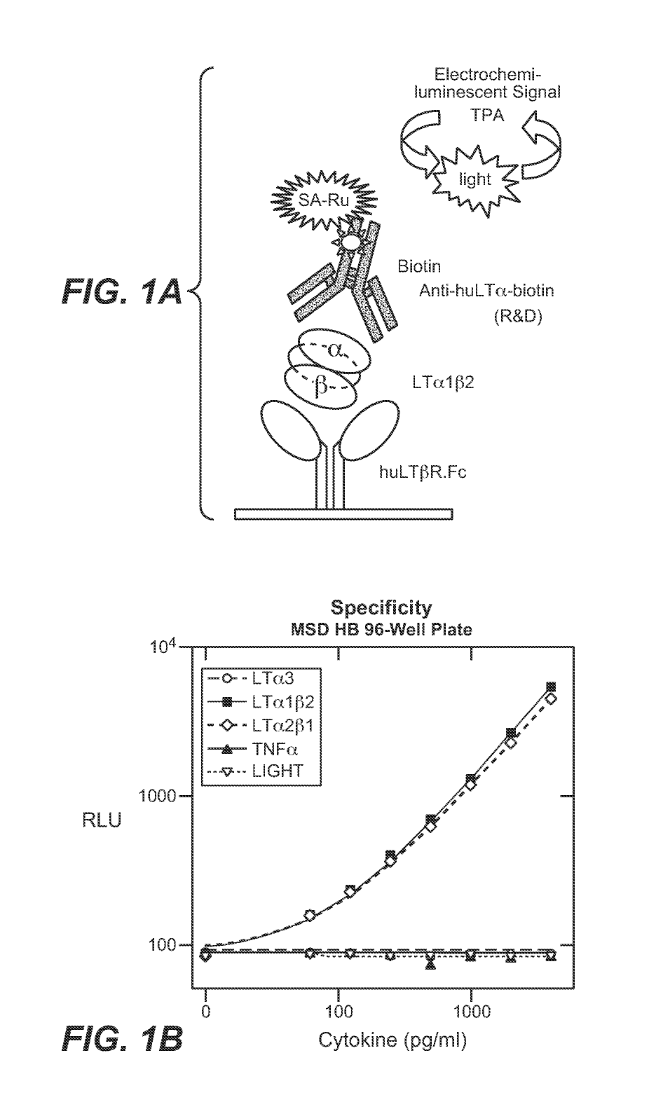

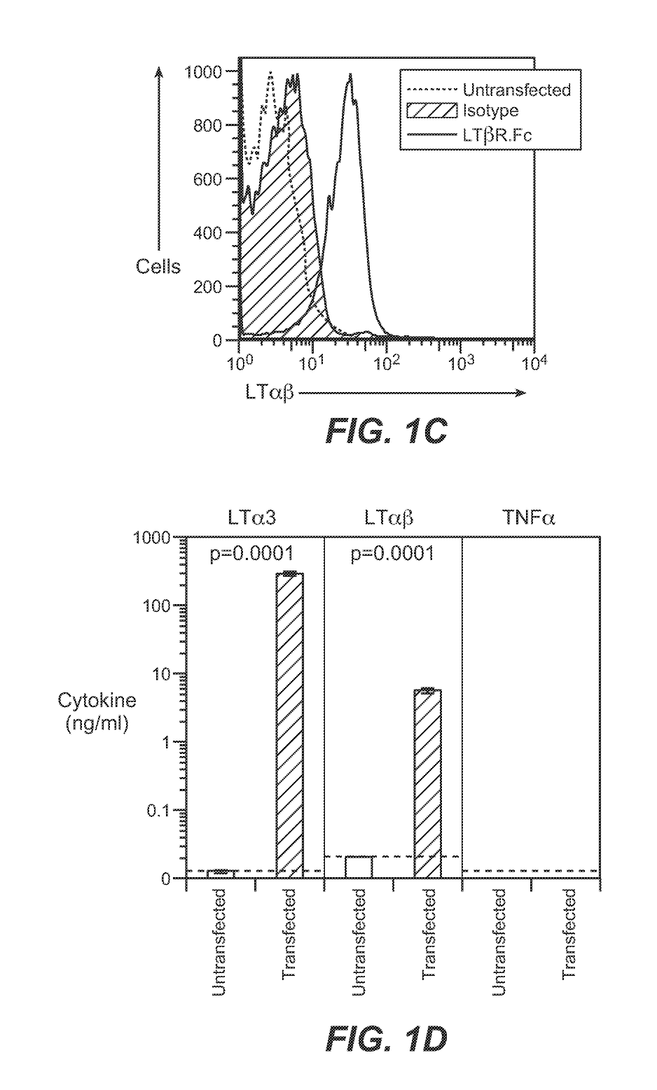

To develop an assay specific for huLTαβ, huLTβR-Fc fusion protein was used for capture. Bound LTαβ was detected with polyclonal anti-LTα-biotin antibody (clone 211, R&D Systems) (assay schematic shown in FIG. 1A). The lower limit of quantitation (LLOQ) for LTαβ is 20 pg/mL in 50% human serum. The present inventors tested the assay for detection of human recombinant LTαβ trimers, as well as other TSF ligands LTα3, and TNF-α (TNFalpha) which do not bind LTβR. LIGHT binds to LTβR, but is not detected by the anti-LTα detection antibody. Detection of various recombinant proteins in this assay is shown in FIG. 1B. TNF-α and LIGHT are not recognized. In addition, the assay does not recognize LTα3, since LTβR requires an LTβ subunit to bind. However, the assay detects both LTα1β2 and LTα2β1.

To verify that this assay could detect native soluble LTαβ, supernatant from a stably transfected 293-huLTαβ (human LTαβ) expressing cell line was assessed. See Example 2, below.

### Example 2

### SolLTαβ is Shed from Activated Lymphocytes In Vitro

This example shows that LTαβ is shed from activated lymphocytes to yield soluble LTαβ (solLTαβ) in the periphery, e.g., in the serum or synovial fluid.

Total human CD4⁺ T cells were isolated from PBMC with a CD4⁺ T cell isolation kit (Miltenyi Biotec). Cells were cultured in complete DMEM media (DMEM supplemented with 10% FBS, 2 mM glutamine, 2 μM 2-ME, 1 mM sodium pyruvate, 100 U/ml penicillin and 100 μg/ml streptomycin) in presence of 5 μg/ml anti-CD3 mAb and 2 μg/ml anti-CD28 mAb. Human Th subset polarization conditions were as follows: Th0: 5 μg/ml anti-hIL-12, 5 μg/ml anti-hIFN-γ 1 μg/ml anti-hIL-4; Th1: 1 ng/ml rhIL-12, 10 ng/ml rhIFN-γ, 1 μg/ml anti-hIL-4; Th2: 5 ng/ml rhIL-4, 5 μg/ml anti-hIL-12, 6 μg/ml anti-hIFN-γ; Th17: 10 ng/ml rhIL-23, 10 μg/ml anti-hIL-12, 10 μg/ml anti-hIFN-γ. T cells were re-stimulated with 5 μg/mL anti-CD3 and 2 μg/mL anti-CD28 with indicated amount of TNFα protease inhibitor 1 (TAPI-1) (Peptides International) or DMSO as control.

At day1 and day2 after re-stimulation, cells were collected for FACS and RNA preparation; culture supernatants were collected for LT and TNF-α quantitation.

Antibodies used for staining were as follows: FITC- or PerCP-anti-CD4, PE-anti-CD25 and Alexa-647-anti-mIgG2a purchased from BD Biosciences; anti-LTalpha, and LTβR-Fc were Alexa-647-conjugated using Alexa Fluor 647 Protein Labeling Kit (Invitrogen). Samples were acquired on a FACSCalibur flow cytometer using CellQuest Pro v.5.1.1 software (BD Biosciences) and data analysis was conducted using FlowJo v6.4.2 software (Tree Star, Inc.). Viable cells were identified by gating based on forward and side scatter. For CFSE labeling of cells, cells were incubated with 5 uM CFSE for 5 min at RT, followed by four washes with PBS. For determination of absolute cell numbers, CaliBRITE APC Beads (BD Biosciences) were added before analyzing samples by flow cytometry, and total cell numbers were determined according to manufacturer's instructions.

SolLTαβ is measured by electrochemiluminescent assays (ECLA) specific for human LTα3 and LTαβ. (See Example 1 (Assays), above.) For LTαβ, human LTβR-Fc fusion protein was used for capture, as this receptor specifically binds LTβ and exclusively captures LT trimers containing one or more β subunits. The assay detected human recombinant LTαβ complexes (LTα1β2 and LTα2β1) similarly, but did not recognize human recombinant LTα3 or other TNF-SF members, TNFα and LIGHT (FIG. 1B). To verify that the assay could detect native soluble LTαβ supernatant from a stable 293-huTαβ expressing cell line was assessed. Expresssion of human LTαβ in 293 cells: 293 cells were transfected with full-length human LTα and human LTβ (human LTα sequence GenBank Ref NM_{—}000595; human LTβ sequence GenBank Ref NM_{—}002341) to generate stable human LTαβ-expressing cell lines. The 293-huLTαβ transfectants express surface LTαβ (FIG. 1C) and supernatants contained high levels of secreted LTα (287±18 ng/mL) and lower levels of soluble LTαβ (5.5±0.51 ng/mL), but no TNFα was detectable as expected (FIG. 1D).

Activated T helper cells secrete soluble LTα3 homotrimers and express LTα1β2 on their surface. LTα1β2 is detected on the surface of cells polarized under Th0, Th1, Th2 or Th17 conditions 24 hours after reactivation, however levels on Th2 cell were significantly lower and completely absent 72 hours post-activation, consistent with previous reports (Gramaglia et al., Lymphotoxin alphabeta is expressed on recently activated naive and Th1-like CD4 cells but is down-regulated by IL-4 during Th2 differentiation. J Immunol 1999; 162(3):1333-8) (Grogan, submitted manuscript). To determine if primary human CD4⁺ LTαβ⁻ lymphocytes also shed soluble LTαβ, we examined supernatants from activated polarized T helper cells on day 2 (FIG. 2A). Analysis of cell culture supernatants for TNF-α, IFN-γ, IL-17, IL-22 and IL-4 confirmed the polarization of these cells (Grogan, submitted manuscript). Soluble LTαβ was detected in culture supernatant whenever LTα3 and TNFα were detected, albeit at lower levels. LTαβ is actively cleaved from activated lymphocytes.

### Example 3

### Activated Human T Cells Shed LTαβ by ADAM17 Protease Cleavage

This example illustrates the role of metalloproteinase cleavage in the shedding of LTαβ by activated T cells.

To determine if LTαβ complexes were shed by metalloproteinase cleavage, the TNFα protease inhibitor 1 (TAPI-1), known to inhibit the cleavage of TNFα by ADAM17 protease, was tested on human CD4′ Th1 cells. Culture supernatants were analyzed for solLTαβ, LTα3 and TNFα one day post re-activation in the presence or absence of TAPI-1 (FIGS. 2A & B). T cells were collected, cultured and polarized as in Example 2 above.

Culture supernatant from resting Th1 polarized lymphocytes contained low levels of solLTαβ (0.10 ng/mL+/−0.06), which increased approximately 8 fold after one day of re-activation (0.78 ng/mL+/−0.40, n=3). Increases in supernatant LTα3 and TNFα were also observed in re-activated cells. TAPI-1 treatment decreased the levels of soluble LTαβ in supernatants of re-activated cells in a dose dependent manner (2-7 fold). Secreted LTα3 was not affected by TAPI-1 treatment, whereas shed TNFα supernatant concentrations were decreased 3-8 fold as expected. Decreased LTαβ in the culture supernatant of TAPI-1 treated cells was not accompanied by an increase in cell surface LT (data not shown), or a change in levels of intracellular LT mRNA (FIG. 2C).

The present Example shows direct evidence that the metalloproteinase (TACE, ADAM17) is one mechanism by which LTαβ complexes are cleaved from the cell surface in a manner similar to other TNF-SF members such as TNF. TAPI-1 (an inhibitor of ADAM17), decreased the concentration of LTαβ detected in supernatants of activated lymphocytes. Therefore, LTαβ may also be cleaved by ADAM17.

### Example 4

### Both LTα and LTβ Subunits are Detected in Activated Human Lymphocyte Culture Supernatant by Western Blotting

To validate the appropriate size of the soluble LTβ cleavage fragments and quantitate the relative ratios of LTα and LTβ subunits, lymphotoxin trimers were immunoprecipitated from activated human lymphocyte culture supernatant using either an anti-LTα antibody or LTβR-Fc.

T cells were collected, cultured and polarized as in Example 2 above.

Agarose beads were conjugated with goat anti-human LTα AF211 or LTβR-Fc using an AminoLink Plus Immobilization Kit 44894 (Pierce, Rockford, Ill.) according to manufacturer's instructions. One mL each of polarized T cell culture supernatants were incubated over night with 100 μL conjugated beads at 4° C. Beads were washed 3× with PBS/0.05% Tween 20/0.5% BSA; immune complexes were denatured and liberated from the beads by incubating 5 minutes at 90° C. in SDS sample buffer (Invitrogen)+5% β mercaptoethanol (Sigma). Recombinant LTα1β2 (R & D Systems, Minneapolis Minn.) was used as standard. Molecular weight markers (Invitrogen), standards, and samples were electrophoresed on a 1.5 mM 4-20% tris-glycerine gel (Invitrogen). Proteins were transferred to a nitrocellulose membrane using iBot (Invitrogen). Membranes were blocked with LI-COR Biosciences (Lincoln, Nebr.) block buffer and probed overnight in LI-COR block buffer containing 100 ng/mL anti-LTα AF211-biotin (R&D Systems) and 500 ng/mL anti-LTβ 1684 (R&D Systems). Blots were washed in PBS+0.1% Tween-20 at room temperature, then probed with secondary reagents; anti-muIg-dye IR800 and streptavidin-dye IR680 (LI-COR), diluted 1:10,000 in LI-COR block buffer at room temperature for 1 hour with agitation, washed as before, and images acquired on LI-COR IR reader.

Blots containing transferred proteins were probed with a mixture of anti-LTα and anti-LTβ specific antibodies and detected with red and green fluorescent dyes, respectively. LTα secreted from human lymphocytes migrated as several glycosylated forms of MW 26-30 KDa, larger than the recombinant protein from R&D, which lacks the first N-terminal 34 amino acids of the native protein. LTβ shed from human lymphocytes also migrated as two glycosylated forms of 28-30 kDa, slightly larger than the recombinant soluble ECD from R&D, which lacks 53 N-terminal amino acids (4 of which are part of the ECD). The size of the LTβ fragment in T cell supernatant is consistent with its cleavage at the membrane surface and release of a glycosylated ECD of 195 amino acids. As expected, anti-LTα-conjugated beads immunoprecipitated a larger amount of LTα than LTβR-Fc-conjugated beads, since anti-LTα immunoprecipitated both homo- and hetero-trimeric complexes from the supernatant (FIG. 2D).

LTαβ is shed into culture superntants in vitro and the assays described herein are specific for LTαβ.

### Example 5

### Increased solLTαβ in Sera of Murine Inflammatory Disease Models

This example illustrates that LTαβ is actively cleaved from activated lymphocytes, is found in the circulation in vivo in preclinical animal models of inflammation and autoimmune disease (CIA and EAE).

EAE Model

Female SJL/J mice were immunized intradermally at the base of the tail with 200 μl of emulsion containing 150 μg of peptide PLP 139-151 in 100 μl of PBS and 100 μl of CFA. On Day 12, animals with a score of 2-4 were randomized into four different treatment groups. Mice were treated with 6 mg/kg anti-ragweed IgG2a monoclonal antibody (control antibody), murine LTβR-Fc or murine TNFRII-Fc in 100 μl PBS, subcutaneously, three times a week for the duration of the study. Animals were evaluated daily for clinical signs using the same grading system as for the transgenic mice.

The severity of experimental autoimmune encephalomyelitis (EAE) in the experimental mouse model, as measured by the clinical score, was reduced versus isotype control by administration of the anti-LTα antibody S5H3, comparable to that seen with the CTLA-4-Fc molecule. On day 70, serum was collected and muLTα was analysed by ELISA. The results thus suggest that the antibody treatment would be efficacious in treating diseases predicted from the EAE model, such as relapsing remitting MS.

Induction of Arthritis and Treatment: CIA Model

In RA the synovial membrane of multiple joints can become inflamed, leading to destruction of joint tissues including bone and cartilage. The synovium of RA can be highly inflammatory in nature and is typically characterized by lymphocyte and mononuclear cell infiltration, T-cell and antigen-pressing cell (APC) activation, B-cell immunoglobulin (Ig) secretion, and pro-inflammatory cytokine production (Potocnik et al., Scand. J. Immunol., 31:213 (1990); Wernick et al., Arthritis Rheum., 28:742 (1985); Ridley et al., Br. J. Rheumatology, 29:84 (1990); Thomas et al., J. Immunol., 152:2613 (1994); Thomas et al., J. Immunol., 156:3074 (1996)). Chronically inflamed synovium is usually accompanied by a massive CD4 T-cell infiltration (Pitzalis et al., Eur. J. Immunol., 18:1397 (1988); Morimoto et al., Am. J. Med., 84:817 (1988)).

Collagen-induced arthritis (CIA) is an animal model for human RA, which resembles human disease, and can be induced in susceptible strains of mice by immunization with heterologous type-II collagen (CII) (Courtenay et al., Nature, 283:665 (1980); Cathcart et al., Lab. Invest., 54:26 (1986)). Both CD4 T cells and antibodies to CII are required to develop CIA. Transfer of anti-CII to naïve animals only leads to partial histo-pathology that is quite different from CIA, and complete symptoms of CIA do not develop (Holmdahl et al., Agents Action, 19:295 (1986)). In contrast, adoptive transfer of both CD4 T cells and anti-CII antibodies from CII-immunized mice to naïve recipients completely reconstitutes the symptoms of classical CIA (Seki et al., J. Immunol., 148:3093 (1992)). Involvement of both T cells and antibodies in CIA is also consistent with histo-pathological findings of inflamed joints in CIA. Thus, agents that block B-cell or T-cell functions, or inhibit pro-inflammatory cytokines induced by T cells, may be efficacious to prevent or treat arthritis. Indeed, depletion of CD4 T cells, blockade of CD40-CD40L interactions, neutralization of TNF-α, or blocking of IL-1 receptors can lead to prevention of CIA in mice (Maini et al., Immunol. Rev., 144:195 (1995); Joosten et al., Arthritis Rheum., 39:797 (1996); Durie et al., Science, 261:1328 (1993)).

In the CIA model used herein, DBA-1J mice were immunized with 100 μg bovine collagen type II in 100 μl of Complete Freund's Adjuvant (CFA) on Day 0 and Day 21 intradermally. At Day 24 post-immunization, mice were randomly divided into treatment groups. Animals were subcutaneously treated either with 6 mg/kg anti-ragweed IgG2a monoclonal antibody (control antibody) or with murine LTβR-Fc in 100 μl PBS. Animals were treated three times weekly for the duration of the study. Limbs of animals were examined daily for signs of joint infiltration using a grading system of 1-4 for each joint, giving a maximum of score 16. Sera was collected on Day 35 for cytokine (e.g., LTα and TNFα) analysis.

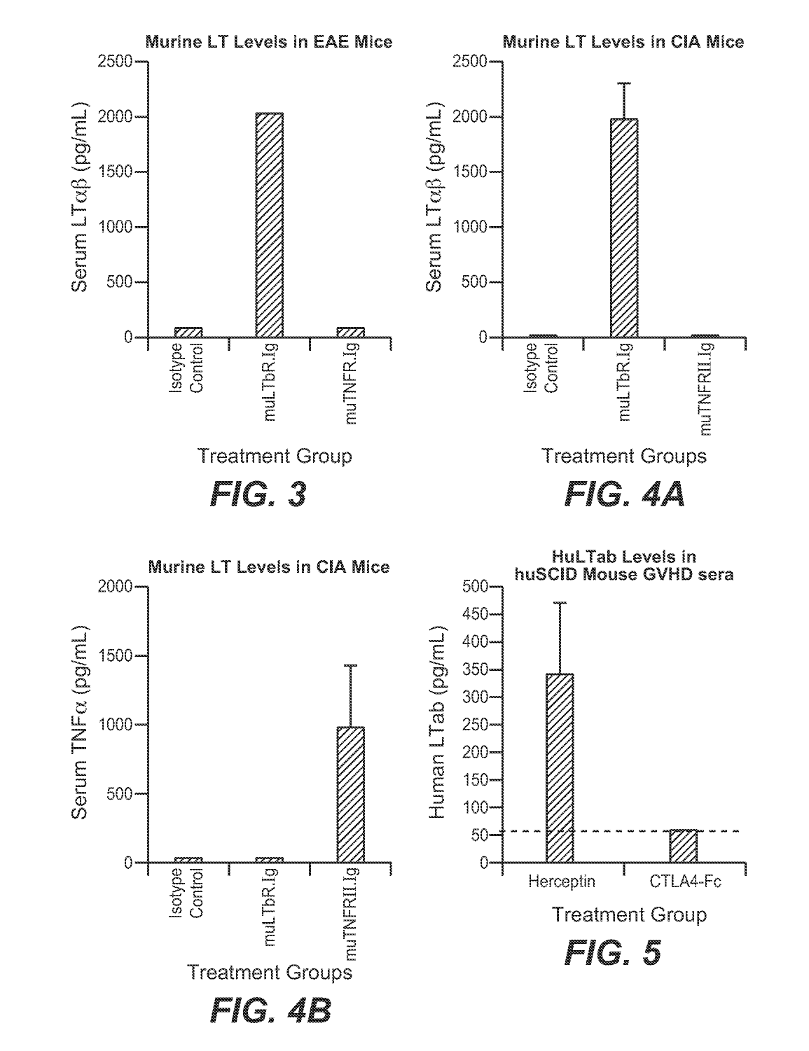

Elevated solLTαβ levels are detected in serum of mice treated for 11 days with muLTβR-Fc (a recombinant murine LTβ receptor conjugated to an immunoglobulin Fc region), but not with isotype control antibody or TNFRII-Fc (EAE, FIG. 3; CIA, FIG. 4A), This suggests the stabilization in the circulation of a ligand, LTαβ, that binds to LTβR-Fc in both murine models. Circulating LT levels were undetectable in normal mice (data not shown) or diseased mice treated with an isotype control antibody. Similarly, increased levels of TNF-α were detected in mice treated with TNFRII-Fc (FIG. 4B).

### Example 6

### Soluble huLTαβ Complexes are Detected in Serum of HuSCID GVHD Model

This example examines LTαβ in a Graft-versus-host disease (GVHD) model and shows that activated human lymphocytes in immunocompetent mice express human LTαβ. GVHD occurs when immunocompetent cells are transplanted into immunosuppressed or tolerant patients. The donor T cells recognize host antigens and become activated, secrete cytokines, proliferate and differentiate into effector cells. This response is known as graft-versus-host-reaction (GVHR). The GVHR response is a multi-organ syndrome and the effects can vary from life-threatening severe inflammation to mild cases of diarrhea and weight loss. GVHD models in mice have been used to model the clinical disorders of acute and chronic GVHR that occur after bone marrow transplantation and autoimmune diseases. A general procedure is described in Current Protocols in Immunology, supra, unit 4.3. In this instance, human PBMCs were purified from LEUKOPACK™ of a normal donor by FICOL™ gradient.

Human peripheral blood mononuclear cells produce graft vs host disease when transplanted into severe combined immune deficient (SCID) mice. SCID mice were reconstituted with human peripheral blood mononuclear cell (PBMC) purified from a leukopack of normal donor. SCID mice transplanted with human leukocytes develop severe graft vs. host disease. All mice (n=10/group) were sub-lethally irradiated with 350 rads using Cesium 137 source. Two hours after irradiation, mice were injected with 50 million human PBMC cells/mouse in 200 ul PBS intravenously. Immediately after cell injection mice were treated IP either with 300 ug of trastuzumab (humanIgG1 isotype control Ab) or CTLA-4-Fc in 100 ul saline 2 times/week. CTLA-4-Fc inhibits T cell activation and reduces the graft vs. host disease response. CTLA-4-Fc was generated in a similar manner as that used to generate mouse LTβR-Fc in Example 1, with the extracellular domain of murine CTLA-4 (position 1 through position 160) cloned into a modified pRK5 expression vector encoding the human IgG1 Fc region downstream of the CTLA-4 sequence. POLYMYXIN™ B 110 mg/liter and Neomycin 1.1 g/liter were added to the drinking water for 5 days post irradiation. Mice were monitored for graft-versus-host-disease (GVHD) as indicated by survival.

Mice were bled and serum collected and analysed by huLTαβ ECLA on day 1.

Soluble human LTαβ was detected in serum of the mice at levels of approximately 350 pg/mL, and was reduced at least 7 fold to undetectable levels in mice treated with CTLA-4-Fc, which suppressed human lymphocyte activation. FIG. 5 shows that no shed human LTαβ complexes were detected in serum of huSCID mice treated with CTLA-4-Fc, however high levels were detected in huSCID mice treated with control antibody (Herceptin). The human LTαβ assay does not cross-react with murine LTαβ.

The huSCID in vivo model of human T cell activation shows elevated circulating LT levels.

### Example 7

### Circulating Peripheral SolLTαβ in Serum and Synovial Fluid of RA Patients

This example shows that LTαβ is shed from activated lymphocytes to yield soluble LTαβ (solLTαβ) and that solLTαβ can be identified in the serum and synovial fluid of RA patients.

Serum samples were collected from healthy human donors and patients fulfilling the 1987 American College of Rheumatology criteria for RA. Synovial fluid samples were collected from patients diagnosed with RA or with osteoarthritis (OA). All healthy controls and patients had given their written informed consent. Blood samples were taken from consenting healthy human donors. Serum was separated from the clotted cellular portion by centrifugation and frozen in aliquots at −80° C.

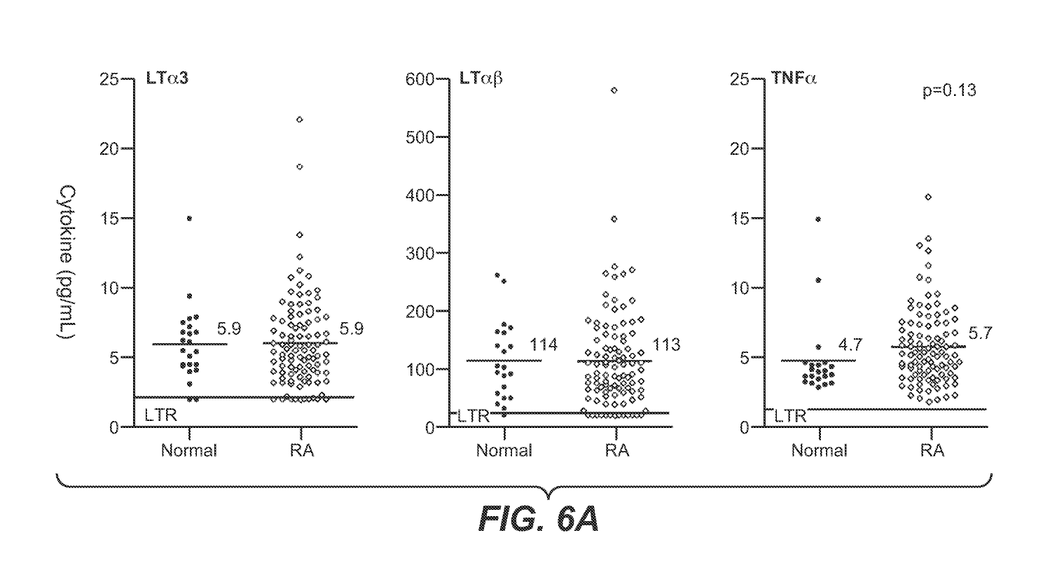

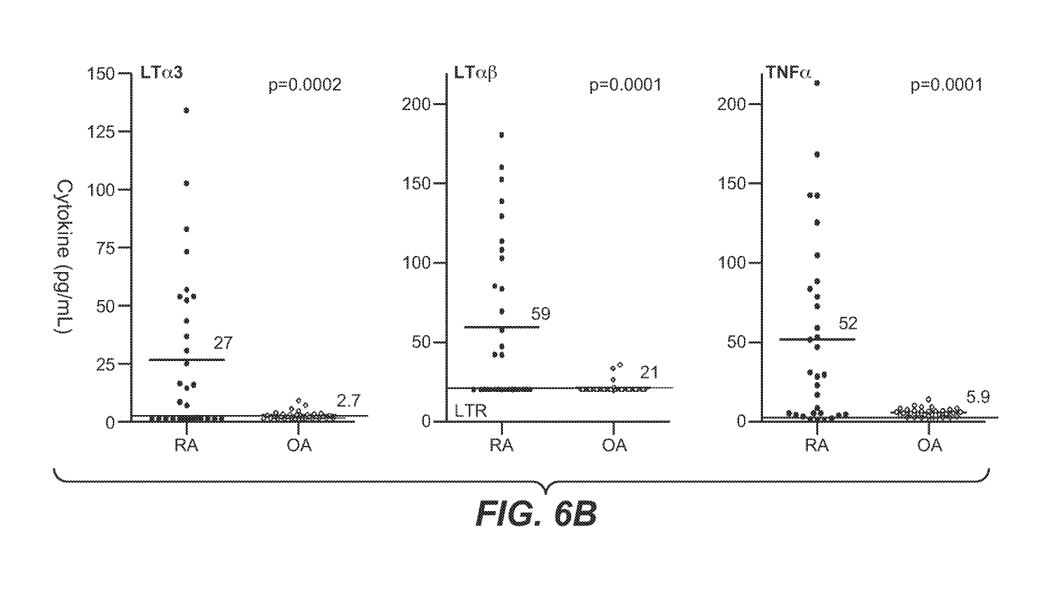

Sera from 23 normal donors and 100 RA patients were analyzed for levels of LTα3, solLTαβ, and TNFα. Using the assays described in Example 1 soluble LTαβ was detected in normal and RA sera at approximately 20 fold higher levels than the soluble LTα3 homotrimer (FIG. 6A). Average solLTαβ, LTα3 and TNFα levels did not significantly differ between normal donors and RA patients, although TNFα levels were elevated in approximately 50% of RA patients.

Pro-inflammatory cytokines are elevated at sites of damaged tissue, therefore, synovial fluid from the inflamed joints of 31 RA patients and 33 osteoarthritis (OA) patients was analyzed for solLTαβ, LTα3, and TNFα levels (FIG. 6B). RA synovial fluid contained an average of 27, 59, and 52 pg/mL of LTα3, solLTαβ, and TNFα, respectively, while OA synovial fluid contained significantly less (average of 2.7, 21, and 5.9 pg/mL of LTα3, solLTαβ, and TNFα, respectively). There was a weak association between LTα3 and solLTαβ levels in RA patient serum (R²=0.37), suggesting that an underlying mechanism may effect the levels of both. No correlation between LTα3 and LTαβ was apparent in synovial fluids of the RA patients analyzed (R²=0.24).

LTαβ is detected in human synovial fluid of arthritis patients. Although LTαβ levels in RA sera are only modestly higher than in healthy controls, levels in synovial fluid are significantly higher than in OA patients. Synovial fluid levels of other inflammatory chemokines and cytokines also tend to be higher in RA vs. OA patients.

### Example 8

### Soluble LTαβ Activates Synovial Fibroblasts from RA Patients

This example examines the ability of the soluble LTαβ heterotrimer to act as a functional proinflammatory cytokine in RA. We assessed its ability, together with the LTα3 isoform, to induce expression of proinflammatory chemokines, cytokines, and adhesion molecules in primary fibroblast-like synoviocytes (FLS) isolated from RA patients.

For this Example soluble LTαβ heterotrimers were expressed in insect cells as follows: The 187 amino acid carboxy-terminal portion of the extra-cellular coding region of the human LTβ gene (c-terminal his tagged) and the 162 amino acid carboxy-terminal portion of the extra-cellular coding region of the human LTα gene (N-terminal flag-tagged) were cloned into the pAcGP67B Baculovirus expression vector (Pharmingen), and viruses were generated with these two constructs. Insect Tni cells were co-infected with both viruses for 3 days in protein free cell culture media at 27° C. The infected cell culture media was purified over Ni-NTA, anti-flag M2 column, then QHP and S200 size exclusion column to purify the LTα1β2 protein.

Total RNA was isolated using the Qiagen RNeasy mini kit (Qiagen, Valencia, Calif.). Real-time RT-PCR was conducted on an ABI 7500 Real-Time PCR system (Applied Biosystems) with Taqman one-step RT-PCR master mix kit following manufacturer's protocol (Applied Biosystems, Foster City, Calif.). Primers and probes used are as follows:

LTα
probe:
(SEQ ID NO: 2)
5′-CAA GGC CAC CTC CTC CCC AC-3′ (FAM-TAMRA);
forward:
(SEQ ID NO: 3)
5′-TCT TCT CTG GGA AAG CCT ACT C-3′;
reverse:
(SEQ ID NO: 4)
5′-CCT CAT GGG CCA GGT AGA-3′.
LTβ
probe:
(SEQ ID NO: 5)
5′-ACG TAC ACC CTC TCG CCC CTC C-3′ (FAM-TAMRA);
forward:
(SEQ ID NO: 6)
5′-ACG GGC CTC TCT GGT ACA-3′;
reverse:
(SEQ ID NO: 7)
5′-CAT ATC GGG GTG ACT GAT GTT-3′.
TNF-α
probe:
(SEQ ID NO: 8)
5′-CTG AGG CCT CTG CTC CCC AGG-3′ (FAM-TAMRA);
forward:
(SEQ ID NO: 9)
5′-TGG TGA CCA ACT GTC ACT CAT-3′;
reverse:
(SEQ ID NO: 10)
5′AAT AGT AGG CCG ATT ACA GAC ACA-3′.
RPL19
probe:
(SEQ ID NO: 11)
5′-CAC AAG CTG AAG GCA GAC AAG GCC C-3′
(FAM, TAMRA);
forward:
(SEQ ID NO: 12)
5′-GCG GAT TCT CAT GGA ACA-3′;
reverse:
(SEQ ID NO: 13)
5′-GGT CAG CCA GGA GCT TCT TG-3′;
IL-8
probe:
(SEQ ID NO:14)
5-AAC TGC ACC TTC ACA CAG AGC TGC-3′ (FAM-BHQ1);
forward
(SEQ ID NO: 15)
5′-CTC TCT TGG CAG CCT TCC TG-3′;
reverse
(SEQ ID NO: 16)
5′-CTA AGT TCT TTA GCA CTC CTT GGC-3′.

The following human primer/probe sets were purchased from ABI (Applied Biosystems, Foster City, Calif.): IL6—Hs99999032_m1; CXCL1—Hs00236937_m1; CXCL2—Hs00236966_m1; ICAM1—Hs99999152_m1; VCAM1—Hs00365485_m1. All assays were done in triplicate and data was normalized to RPL19.

Isolation of primary synovial fibroblasts was performed as follows: synovial tissue obtained from RA patients fulfilling the 1987 ACR criteria was processed 24 hours post-biopsy. Tissue was digested in 50 μg/mL collagenase type VIII in RPMI media for 90 minutes at 37° C. with agitation. The digested tissue mixture was filtered over a 70 μm mesh cell strainer and washed twice in DMEM. Cells were counted and plated at 1×10⁶ cells/mL in DMEM. After 24 hours of culture, non-adherent cells were aspirated and fresh media was replaced on adherent cells. Cells were passaged at 90% confluence and passage number for all experiments did not exceed 5. The synovial fibroblast cultures were >99% pure and free of macrophage contamination as assessed by CD14 staining with flow cytometry.

Cultured FLS were incubated for 6 hours at 37° C. with either 300 ng/mL LTαβ or 100 ng/mL LTα3 or media alone (control). In a second experiment, FLS were stimulated with LTαβ or LTα3 alone or in the presence of 25 μg/mL LTβR-Fc or TNFRII-Fc. In both experiments, total RNA was purified from the cells and quantitative PCR performed for the genes shown in FIG. 4 using the above nucleotide sequences.

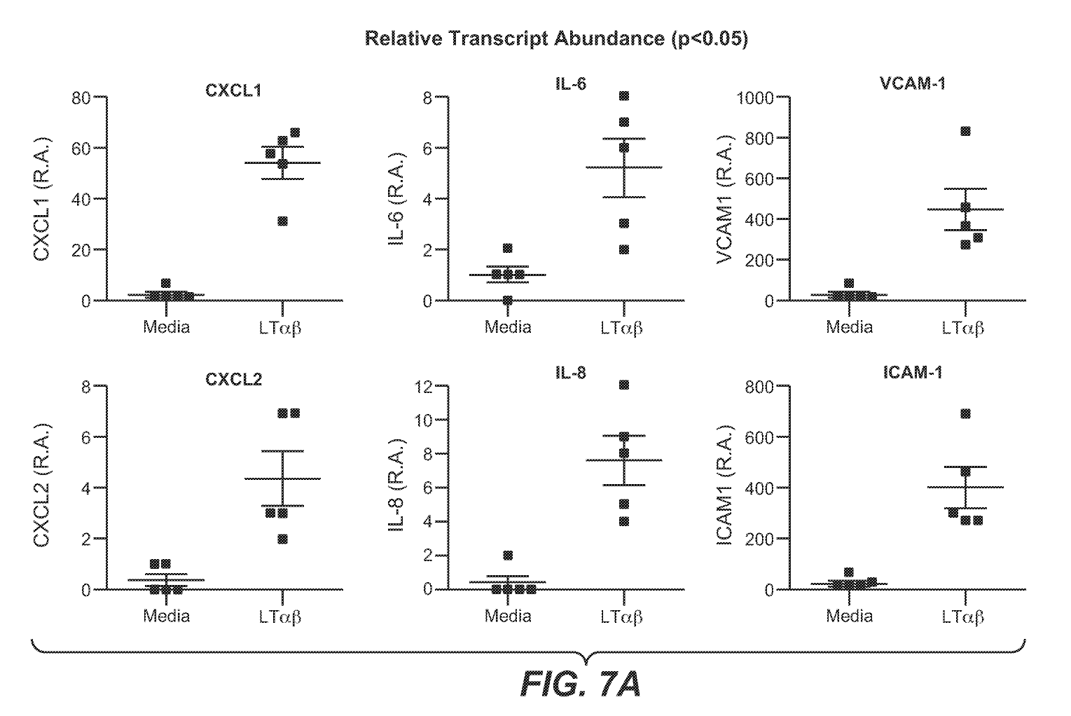

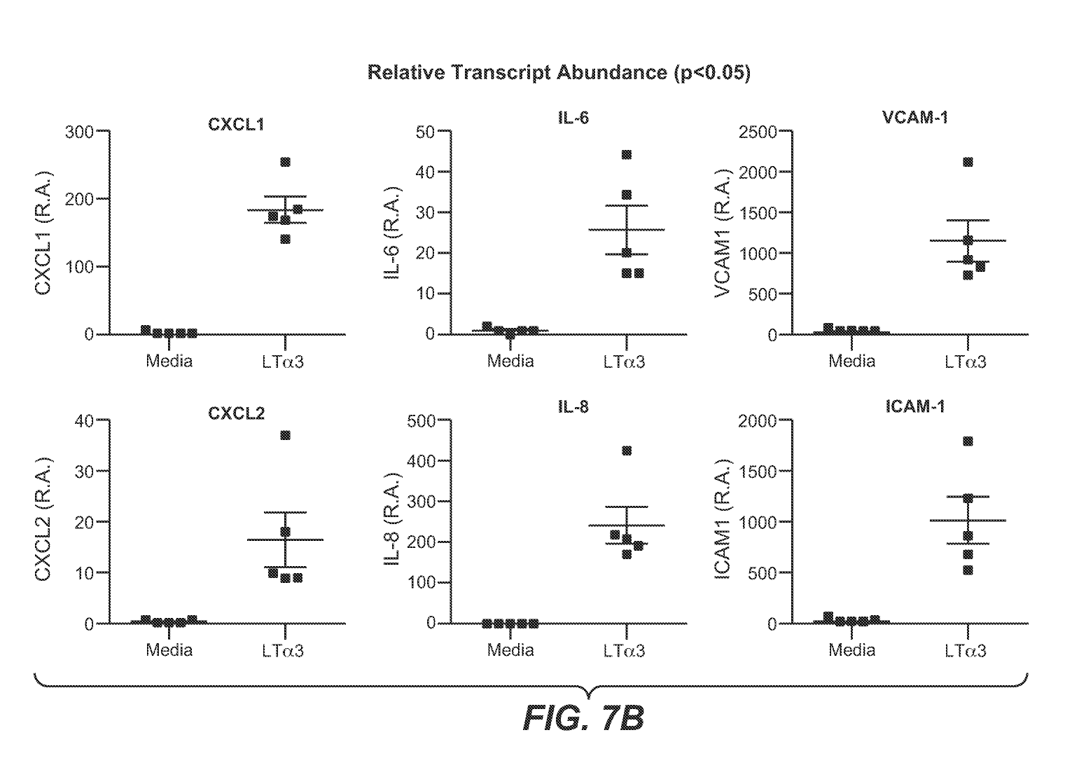

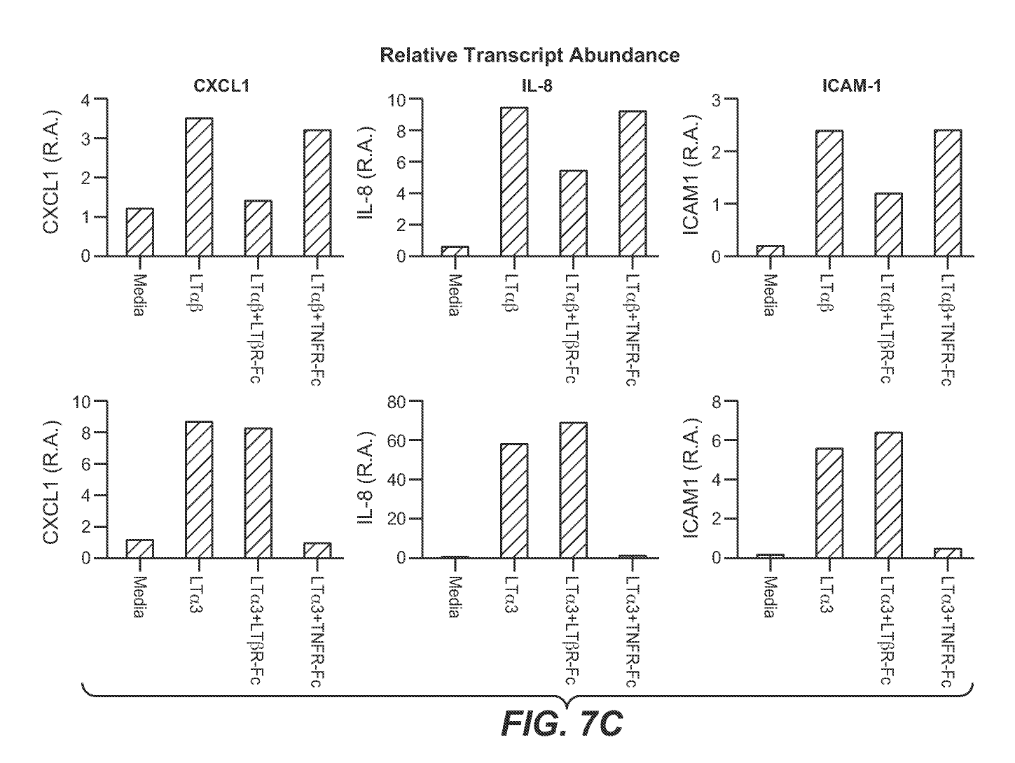

Culture of FLS with LTαβ resulted in the rapid induction of transcripts for CXCL1 (GROα), CXCL2 (GROβ), IL-6, IL-8, VCAM-1 and ICAM-1 (FIG. 7A). LTα3 was also able to induce these genes, but with an increased biological potency (FIG. 7B). To confirm the specificity of these cytokine/cytokine receptor interactions, we also performed stimulation of RA FLS with LTαβ or LTα3 in the presence of LTβR-Fc or TNFRII-Fc. As shown in FIG. 7C, LTβR-Fc but not TNFRII-Fc blocked proinflammatory gene expression induced by LTαβ, while TNFRII-Fc but not LTβR-Fc blocked gene expression induced by LTα3. Therefore, both LTαβ and solLTαβ trimeric isoforms act as cytokines and drive expression of proinflammatory genes in primary RA FLS.

### Example 9

### Statistical Methods

This example shows methods useful in biomarker prediction.

The statistical tasks can comprise the following steps:

1. Pre-selection of candidate biomarkers

2. Pre-selection of relevant clinical efficacy response predictive covariates

3. Selection of biomarker prediction functions at a univariate level

4. Selection of biomarker prediction functions including clinical covariates at a univariate level

5. Selection of biomarker prediction functions at a multivariate level

6. Selection of biomarker prediction functions including clinical covariates at a multivariate level

The following text details the different steps:

1: Pre-selection of candidate biomarkers: The statistical pre-selection of candidate biomarkers is oriented towards the strength of association with measures of clinical benefit. For this purpose the different clinical endpoints may be transformed in derived surrogate scores, as, e.g., an ordinal assignment of the degree of clinical benefit scores regarding TTP that avoid censored observations. These surrogate transformed measures can be easily used for simple correlation analysis, e.g. by the non-parametric Spearman rank correlation approach. An alternative is to use the biomarker measurements as metric covariates in time-to-event regression models, as, e.g., Cox proportional hazard regression. Depending on the statistical distribution of the biomarker values, this step may require some pre-processing, as, for example, variance-stabilizing transformations and the use of suitable scales or, alternatively, a standardization step such as using percentiles instead of raw measurements. A further approach is inspection of bivariate scatter plots, for example, by displaying the scatter of (x-axis=biomarker value, y-axis=measure of clinical benefit) on a single-patient basis. Some non-parametric regression line as achieved, for example, by smoothing splines can be useful to visualize the association of biomarker and clinical benefit.

The goal of these different approaches is the pre-selection of biomarker candidates that show some association with clinical benefit in at least one of the benefit measures employed, while results for other measures are not contradictory. When there are available control groups, then differences in association of biomarkers with clinical benefit in the different arms could be a sign of differential prediction that makes the biomarker(s) eligible for further consideration.

2: Pre-selection of relevant clinical efficacy response predictive covariates: The statistical pre-selection of clinical covariates as defined herein parallels the approaches for pre-selecting biomarkers and is also oriented towards the strength of association with measures of clinical benefit. So in principle the same methods apply as considered under 1 above. In addition to statistical criteria, criteria from clinical experience and theoretical knowledge may apply to pre-select relevant clinical covariates.

The predictive value of clinical covariates could interact with the predictive value of the biomarkers. They will be considered for refined prediction rules, if necessary.

3: Selection of biomarker prediction functions at a univariate level: The term “prediction function” will be used in a general sense to mean a numerical function of a biomarker measurement that results in a number scaled to imply the target prediction.

A simple example is the choice of the Heaviside function for a specific cutoff c and a biomarker measurement x, where the binary prediction A or B is to be made, then If H (x−c)=0, then predict A. If H (x−c)=1, then predict B.

This is probably the most common way of using univariate biomarker measurements in prediction rules. The definition of “prediction function” as noted above includes referral to an existing training data set that can be used to explore the prediction possibilities. Different routes can be taken to achieve a suitable cutoff c from the training set. First, the scatterplot with smoothing spline mentioned under 1 can be used to define the cutoff. Alternatively, some percentile of the distribution could be chosen, e.g., the median or a quartile. Cutoffs can also be systematically extracted by investigating all possible cutoffs according to their prediction potential with regard to the measures of clinical benefit. Then, these results can be plotted to allow for an either manual selection or to employ some search algorithm for optimality. This can be realized based on certain clinical endpoints using a Cox model, wherein at each test cutoff the biomarker is used as a binary covariate. Then the results for the clinical endpoints can be considered together to chose a cutoff that shows prediction in line with both endpoints.

Another uncommon approach for choosing a prediction function can be based on a fixed-parameter Cox regression model obtained from the training set with biomarker values (possibly transformed) as covariate. A further possibility is to base the decision on some likelihood ratio (or monotonic transform of it), where the target probability densities are pre-determined in the training set for separation of the prediction states. Then the biomarker would be plugged into some function of predictive criteria.

4: Selection of biomarker prediction functions including clinical covariates at a univariate level: Univariate refers to using only one biomarker—with regard to clinical covariates, this can be a multivariate model. This approach parallels the search without clinical covariates, except that the methods should allow for incorporating the relevant covariate information. The scatterplot method of choosing a cutoff allows only a limited use of covariates, e.g., a binary covariate could be color coded within the plot. If the analysis relies on some regression approach, then the use of covariates (also many of them at a time) is usually facilitated. The cutoff search based on the Cox model described under 3 above allows for an easy incorporation of covariates and thereby leads to a covariate adjusted univariate cutoff search. The adjustment by covariates may be done as covariates in the model or via the inclusion in a stratified analysis.

Also the other choices of prediction functions allow for the incorporation of covariates.

This is straightforward for the Cox model choice as prediction function. This includes the option to estimate the influence of covariates on an interaction level, which means that, e.g., for different age groups different predictive criteria apply.

For the likelihood ratio type of prediction functions, the prediction densities must be estimated including covariates. For this purpose, the methodology of multivariate pattern recognition can be used or the biomarker values can be adjusted by multiple regression on the covariates (prior to density estimation).

The CART technology (Classification and Regression Trees; Breiman et al. (Wadsworth, Inc.: New York, 1984) can be used for this purpose, employing a biomarker (raw measurement level) plus clinical covariates and utilizing a clinical benefit measure as response. Cutoffs are searched and a decision-tree type of function will be found involving the covariates for prediction. The cutoffs and algorithms chosen by CART are frequently close to optimal and may be combined and unified by considering different clinical benefit measures.

5: Selection of biomarker prediction functions at a multivariate level: When there are several biomarker candidates that maintain their prediction potential within the different univariate prediction function choices, then a further improvement may be achieved by combinations of biomarkers, i.e., considering multivariate prediction functions.

Based on the simple Heaviside function model, combinations of biomarkers may be evaluated, e.g., by considering bivariate scatterplots of biomarker values where optimal cutoffs are indicated. Then a combination of biomarkers can be achieved by combining different Heaviside function by the logical “AND” and “OR” operators to achieve an improved prediction.

The CART technology can be used for this purpose, employing multiple biomarkers (raw measurement level) and a clinical benefit measure as response, to achieve cutoffs for biomarkers and decision-tree type of functions for prediction. The cutoffs and algorithms chosen by CART are frequently close to optimal and may be combined and unified by considering different clinical benefit measures.

The Cox-regression can be employed on different levels. A first way is to incorporate the multiple biomarkers in a binary way (i.e., based on Heaviside functions with some cutoffs). The other option is to employ biomarkers in a metric way (after suitable transformations), or a mixture of the binary and metric approach. The evolving multivariate prediction function is of the Cox type as described under 3 above.

The multivariate likelihood ratio approach is difficult to implement, but presents another option for multivariate prediction functions.

6: Selection of biomarker prediction functions including clinical covariates at a multivariate level: When there are relevant clinical covariates, then a further improvement may be achieved by combining multiple biomarkers with multiple clinical covariates. The different prediction function choices will be evaluated with respect to the possibilities to include clinical covariates.

Based on the simple logical combinations of Heaviside functions for the biomarkers, further covariates may be included to the prediction function based on the logistic regression model obtained in the training set.

The CART technology and the evolving decision trees can be easily used with additional covariates, which would include these in the prediction algorithm.

All prediction functions based on the Cox-regression can use further clinical covariates. The option exists to estimate the influence of covariates on an interaction level, which means that, e.g., for different age groups different predictive criteria apply.

The multivariate likelihood ratio approach is not directly extendible to the use of additional covariates.

What is claimed is:

1. A method of assessing whether a rheumatoid arthritis (RA) patient is responsive to treatment with a lymphotoxin (LT) antagonist, the method comprising:
a) determining the amount of soluble LTalpha-beta (solLTαβ) in a sample obtained from an RA patient treated with the LT antagonist, as compared to the amount of solLTαβ in a sample obtained from an untreated RA patient,
b) wherein a higher or lower amount of solLTαβ in the sample from the treated RA patient as compared to the amount of solLTαβ in the sample from the untreated patient is indicative of the treated RA patient's responsiveness to treatment with the LT antagonist.

2. A method of monitoring the efficacy of treatment with an LT antagonist in an RA patient, the method comprising:
a) determining the amount of soluble LTalpha-beta (solLTαβ) in a sample obtained from an RA patient treated with the LT antagonist, as compared to the amount of solLTαβ in a sample obtained from an untreated RA patient,
b) wherein a higher or lower amount of solLTαβ in the sample from the treated RA patient as compared to the amount of solLTαβ in the sample from the untreated patient is indicative of the efficacy of treatment with an LT antagonist in the RA patient.

3. A method of identifying an LT antagonist as a therapeutic agent effective to treat rheumatoid arthritis (RA) in a patient subpopulation, the method comprising:
a) determining a correlation between efficacy of the LT antagonist and the presence of an amount of soluble LTαβ in samples from the patient subpopulation as compared to the amount of solLTαβ in a sample obtained from an untreated RA patient,
b) wherein a higher or lower amount of solLTαβ in the samples from the patient subpopulation as compared to the amount of solLTαβ in the sample from the untreated patient is indicative that the LT antagonist is effective to treat rheumatoid arthritis (RA) in the patient subpopulation.

4. A method of identifying a patient subpopulation for which an LT antagonist is effective to treat rheumatoid arthritis (RA), the method comprising:
a) determining a correlation between efficacy of the LT antagonist and the presence of an amount of soluble LTαβ in samples from the patient subpopulation as compared to the amount of solLTαβ in a sample obtained from an untreated RA patient,
b) wherein a higher or lower amount of solLTαβ in the samples from the patient subpopulation as compared to the amount of solLTαβ in the sample from the untreated patient is indicative that the LT antagonist is effective to treat rheumatoid arthritis (RA) in the patient subpopulation.

5. A method of predicting responsiveness of an RA patient to treatment with an LT antagonist, the method comprising:
a) determining the amount of soluble LTalpha-beta (solLTαβ) in a sample obtained from an RA patient after treatment with the LT antagonist, as compared to the amount of solLTαβ in a sample obtained from an untreated RA patient,
b) wherein a higher or lower amount of solLTαβ in the sample from the treated RA patient as compared to the amount of solLTαβ in the sample from the untreated patient is predictive of responsiveness in the RA patient to treatment with an LT antagonist.

6. A method of monitoring responsiveness of an RA patient to treatment with an LT antagonist, the method comprising:
a) determining the amount of soluble solLTαβ in a sample obtained from the RA patient after treatment with the LT antagonist, as compared to the amount of solLTαβ in a sample obtained from the RA patient before the LT antagonist treatment,
b) wherein a higher or lower amount of solLTαβ in the sample obtained after treatment as compared to the amount of solLTαβ in the sample obtained before treatment is indicative of the responsiveness to treatment with the LT antagonist.

7. A method of modifying treatment of an RA patient with an LT antagonist, the method comprising:
a) determining the amount of solLTαβ in a sample obtained from the RA patient after treatment with the LT antagonist, as compared to the amount of solLTαβ in a sample obtained from the RA patient before the LT antagonist treatment, wherein a higher or lower amount of solLTαβ in the sample obtained after treatment as compared to the amount of solLTαβ in the sample obtained before treatment is indicative of the responsiveness to treatment with the LT antagonist, and
b) adjusting the amount of an LT antagonist administered to the patient based on the higher or lower amount of solLTαβ.

8. A method of designing a treatment with an LT antagonist for an RA patient, the method comprising:
a) determining the amount of solLTαβ in a sample obtained from the RA patient after treatment with the LT antagonist, as compared to the amount of solLTαβ in a sample obtained from the RA patient before the LT antagonist treatment, wherein a higher or lower amount of solLTαβ in the sample obtained after treatment as compared to the amount of solLTαβ in the sample obtained before treatment is indicative of the responsiveness to treatment with the LT antagonist, and
b) designing the treatment with an LT antagonist for an RA patient based on the higher or lower amount of solLTαβ, wherein the designing comprises an adjustment of the amount of LT antagonist administered to the patient.

9. A method of predicting prognosis of an autoimmune disease in a patient, the method comprising:
a) determining the amount of solLTαβ in a sample obtained from the patient after treatment with an LT antagonist, as compared to the amount of solLTαβ in a sample obtained from the patient before the LT antagonist treatment, wherein a higher or lower amount of solLTαβ in the sample obtained after treatment as compared to the amount of solLTαβ in the sample obtained before treatment is indicative of the prognosis of the disease, and
b) adjusting the amount of the LT antagonist administered to the patient based on the higher or lower amount of solLTαβ.

10. A method of monitoring responsiveness of patient with rheumatoid arthritis (RA), to treatment with a lymphotoxin (LT) antagonist, the method comprising:
a) determining the amount of solLTαβ in a sample obtained from the RA patient after treatment with the LT antagonist, as compared to the amount of solLTαβ in a sample obtained from the RA patient before the LT antagonist treatment, and
b) repeating step (a),
wherein a sustained change in the amount of solLTαβ in the sample obtained after treatment as compared to the amount of solLTαβ in the sample obtained before treatment is indicative of the responsiveness to treatment with the LT antagonist.

11. A method of modifying a treatment of an RA patient with an LT antagonist, the method comprising:
a) determining the amount of solLTαβ in a sample obtained from the RA patient after treatment with the LT antagonist, as compared to the amount of solLTαβ in a sample obtained from the RA patient before the LT antagonist treatment,
b) repeating step (a), wherein a sustained change in the amount of solLTαβ in the sample obtained after treatment as compared to the amount of solLTαβ in the sample obtained before treatment is indicative of the responsiveness to treatment with the LT antagonist, and
c) adjusting the amount of an LT antagonist administered to the patient based on the sustained change in the amount of solLTαβ.

12. A method of diagnosing or predicting an autoimmune disease in a patient, the method comprising:
determining the amount of solLTαβ in a sample obtained from the patient after treatment with an LT antagonist, as compared to the amount of solLTαβ in a sample obtained from the patient before the LT antagonist treatment,
wherein a higher or lower amount of solLTαβ in the sample obtained after treatment as compared to the amount of solLTαβ in the sample obtained before treatment is indicative of the disease in the patient.

13. A method of diagnosing or predicting a patient at risk for an autoimmune disease, the method comprising:
determining the amount of solLTαβ in a sample obtained from the patient after treatment with an LT antagonist, as compared to the amount of solLTαβ in a sample obtained from the patient before the LT antagonist treatment,
wherein a higher or lower amount of solLTαβ in the sample obtained after treatment as compared to the amount of solLTαβ in the sample obtained before treatment is indicative of the disease in the patient.

14. The method of claim 12 or 13, wherein the patient is treated with a lymphotoxin (LT) antagonist.

15. The method of claim 12 or 13, wherein the amount of soluble LTαβ (solLTαβ) is in the range of 10-500 pg/mL.

16. The method of any one of claims 1-11, wherein the amount of soluble LTαβ (solLTαβ) is in a range about 1-10,000 pg/mL in the patient serum.

17. The method of any one of claims 1-11, wherein the amount of soluble LTαβ (solLTαβ) is in a range about 25-800 pg/mL in the patient serum.

18. The method of any one of claims 1-11, wherein the amount of soluble LTαβ (solLTαβ) is in the range of 20-400 pg/ml in the patient synovial fluid or tissue.

19. The method of any one of claims 1-11, wherein the amount of soluble LTαβ (solLTαβ) is measured within 24 hours, 50 days or 100 days after receiving a first dose of the lymphotoxin (LT) antagonist.

20. The method of any one of claims 1-16, wherein the antagonist is an antibody or immunoadhesin.

21. The method of any one of claims 1-16, wherein the antagonist is an antibody.

22. The method of any one of claims 1-16, wherein the antibody is a chimeric, humanized, or human antibody.

23. The method of claim 18, wherein the antibody is an anti-lymphotoxin alpha (LTα) antibody.

24. The method of any one of claims 1-16, wherein the antagonist is not conjugated with a cytotoxic agent.

25. The method of any one of claims 1-16, wherein the antagonist is conjugated with a cytotoxic agent.

26. The method of any one of claims 1-16, wherein the patient has never been previously administered a medicament for the rheumatoid arthritis.

27. The method of any one of claims 1-16, wherein the patient has been previously administered at least one medicament for the rheumatoid arthritis.

28. The method of claim 25, wherein the patient was not responsive to the at least one medicament that was previously administered.

29. The method of claim 26, wherein the previously administered medicament or medicaments are an immunosuppressive agent, cytokine antagonist, integrin antagonist, corticosteroid, analgesic, a disease-modifying anti-rheumatic drug (DMARD), or a non-steroidal anti-inflammatory drug (NSAID).

30. The method of any one of claims 1-16, wherein the lymphotoxin antagonist is administered intravenously.

31. The method of any one of claims 1-16, wherein the lymphotoxin antagonist is administered subcutaneously.

32. The method of any one of claims 1-16, wherein at least about three months after the lymphotoxin antagonist treatment, an imaging test is given that measures a reduction in bone or soft tissue joint damage as compared to a baseline prior to the treatment, and the amount of the lymphotoxin antagonist administered is effective in achieving a reduction in the joint damage.

33. The method of claim 30, wherein the test measures a total modified Sharp score.

34. The method of claim 1 wherein the lymphotoxin antagonist is administered without any other medicament to treat the RA.

35. The method any claim 1 wherein the lymphotoxin antagonist treatment further comprises administering an effective amount of one or more second medicaments with the lymphotoxin antagonist, wherein the lymphotoxin antagonist is a first medicament.

36. The method of claim 35, wherein the second medicament is more than one medicament.

37. The method of claim 35, wherein the second medicament is an immunosuppressive agent, a disease-modifying anti-rheumatic drug (DMARD), a pain-control agent, an integrin antagonist, a non-steroidal anti-inflammatory drug (NSAID), a cytokine antagonist, a bisphosphonate, or a combination thereof.

38. The method of claim 37, wherein the second medicament is a DMARD.

39. The method of claim 38, wherein the DMARD is selected from the group consisting of auranofin, chloroquine, D-penicillamine, injectable gold, oral gold, hydroxychloroquine, sulfasalazine, myocrisin and methotrexate.

40. The method of claim 37, wherein the second medicament is a NSAID.

41. The method of claim 40, wherein the NSAID is selected from the group consisting of: fenbufen, naprosyn, diclofenac, etodolac, indomethacin, aspirin and ibuprofen.

42. The method of claim 37, wherein the immunosuppressive agent is selected from the group consisting of etanercept, infliximab, adalimumab, leflunomide, anakinra, azathioprine, and cyclophosphamide.

43. The method of claim 35, wherein the second medicament is selected from the group consisting of anti-alpha4, etanercept, infliximab, etanercept, adalimumab, kinaret, efalizumab, osteoprotegerin (OPG), anti-receptor activator of NFκB ligand (anti-RANKL), anti-receptor activator of NFκB-Fc (RANK-Fc), pamidronate, alendronate, actonel, zolendronate, clodronate, methotrexate, azulfidine, hydroxychloroquine, doxycycline, leflunomide, sulfasalazine (SSZ), prednisolone, interleukin-1 receptor antagonist, prednisone, and methylprednisolone.

44. The method of claim 35, wherein the second medicament is selected from the group consisting of infliximab, an infliximab/methotrexate (MTX) combination, MTX, etanercept, a corticosteroid, cyclosporin A, azathioprine, auranofin, hydroxychloroquine (HCQ), combination of prednisolone, MTX, and SSZ, combinations of MTX, SSZ, and HCQ, the combination of cyclophosphamide, azathioprine, and HCQ, and the combination of adalimumab with MTX.

45. The method of claim 42, wherein the corticosteroid is prednisone, prednisolone, methylprednisolone, hydrocortisone, or dexamethasone.

46. The method of claim 42, wherein the second medicament is MTX.

47. The method of claim 44, wherein the MTX is administered perorally or parenterally.

48. The method of any one of claims 1-16, wherein the arthritis is early rheumatoid arthritis or incipient rheumatoid arthritis.

49. The method of any one of claims 1-16, wherein the patient has exhibited an inadequate response to one or more anti-tumor necrosis factor (TNF) inhibitors.

50. The method of any one of claims 1-16, wherein the amount of the soluble lymphotoxin alpha-beta (solLTαβ) is measured within 24 hours, 50 days or 100 days after receiving a first dose of the lymphotoxin (LT) antagonist.

51. The method of any one of claims 1-16 further comprising re-treating the patient by administering an effective amount of the lymphotoxin antagonist to the patient, wherein the re-treatment is commenced at least about 24 weeks after the first administration of the antagonist.

52. The method of claim 49 wherein the amount of the lymphotoxin antagonist administered upon each administration thereof is effective to achieve a continued or maintained reduction in joint damage.

53. The method of claim 49 wherein a further re-treatment is commenced with an effective amount of the lymphotoxin antagonist.

54. The method of claim 51 wherein the further re-treatment is commenced at least about 24 weeks after the second administration of the antagonist.

55. The method of claim 49 wherein joint damage has been reduced after the re-treatment.

56. The method of claim 49 wherein no clinical improvement is observed in the patient at the time of the testing after the re-treatment.

57. A method of treating rheumatoid arthritis in a patient comprising first administering an effective amount of a lymphotoxin antagonist to the patient to treat the rheumatoid arthritis, provided that a sample from the patient contains an amount of a lymphotoxin (LT) that is greater than the amount of LT in a control wherein the greater amount is indicative of responsiveness of the patient to the lymphotoxin antagonist treatment and at least about 24 weeks after the first administration of the antagonist re-treating the patient by administering an effective amount of the lymphotoxin antagonist to the patient, wherein no clinical improvement is observed in the patient at the time of the testing after the first administration of the lymphotoxin antagonist.

58. The method of claim 55 wherein the test sample is serum, synovial tissue or synovial fluid.

59. A method for monitoring LTαβ processing in vivo, said method comprising detecting the presence of solLTαβ in a tissue specimen or fluid sample from a patient having RA.

60. A method for identifying soluble LTalpha-beta (solLTαβ) production inhibitors, said method comprising:
a) detecting the amount of solLTαβ in a specimen from a test subject/patient having RA and to which a test compound has been administered; and
b) comparing the detected amount of solLTαβ with a control amount of solLTαβ produced in the absence of said test compound.

61. An isolated soluble LT comprising at least one LTα subunit and at least one LTβ subunit wherein the at least one LTβ subunit has been cleaved anywhere between the end of the transmembrane region and about amino acid 95 of SEQ ID NO:2 in U.S. Pat. No. 5,661,004.

62. The isolated soluble LT of claim 61 wherein the end of the LTβ transmembrane region is at about amino acid 44 of SEQ ID NO:2 in U.S. Pat. No. 5,661,004.

63. A method of assessing whether a rheumatoid arthritis (RA) patient is responsive to treatment with a lymphotoxin (LT) antagonist, the method comprising assessing the RA patient's responsiveness based on a different amount of soluble LTalpha-beta (solLTab) in a sample of biological fluid obtained from the RA patient treated with the LT antagonist relative to solLTab amounts in an untreated RA patient, wherein the different amount is indicative of the RA patient's responsiveness to treatment with the LT antagonist.

64. The method of claim 63 wherein the assessing step is preceded by the step of testing the amount of soluble LTalpha-beta (solLTab) in a sample of biological fluid obtained from the RA patient treated with the LT antagonist.

65. The method of claim 64, wherein said testing is implemented using an apparatus adapted to determine the amount of solLTab.

66. The method of claim 64, wherein said testing is performed by using a software program executed by a suitable processor.

67. The method of claim 66, wherein the program is embodied in software stored on a tangible medium.

68. The method of claim 67, wherein the tangible medium is selected from the group consisting of a CD-ROM, a floppy disk, a hard drive, a DVD, and a memory associated with the processor.

69. The method of any one of claims 64 to 68, further comprising the step of preparing a report recording the results of said testing or the diagnosis.

70. The method of claim 69, wherein said report is recorded or stored on a tangible medium.

71. The method of claim 70, wherein the tangible medium is paper.

72. The method of claim 70, wherein the tangible medium is selected from the group consisting of a CD-ROM, a floppy disk, a hard drive, a DVD, and a memory associated with the processor.

73. The method of any one of claims 64 to 68, further comprising the step of communicating the results of said diagnosis to an interested party.

74. The method of claim 73, wherein the interested party is the patient or the attending physician.

75. The method of claim 73, wherein the communication is in writing, by email, or by telephone.

76. A report comprising results of and/or assessment based on a test comprising:
a) testing the level of soluble LTalpha-beta (solLTαβ) in a sample of biological fluid obtained from an RA patient treated with an LT antagonist; and
b) assessing the patient's responsiveness to treatment with an LT antagonist based on a different level of soluble LTalpha-beta (solLTαβ) in the sample relative to a level of solLTab in an untreated patient,
wherein the different level is indicative of the RA patient's responsiveness to treatment with the LT antagonist.

77. A tangible medium storing results of and/or assessment based on a test comprising:
a) testing the level of soluble LTalpha-beta (solLTαβ) in a sample of biological fluid obtained from an RA patient treated with an LT antagonist; and
b) assessing the patient's responsiveness to treatment with an LT antagonist based on a different level of soluble LTalpha-beta (solLTαβ) in the sample relative to a level of solLTab in an untreated patient,
wherein the different level is indicative of the RA patient's responsiveness to treatment with the LT antagonist.
